(19) United States
(12) Patent Application Publication
(10) Pub. No.: US 2003/0037357 A1
(43) Pub. Date: Feb. 20, 2003

(54) Plant acyl-CoA synthetases

(75) Inventors: Jay M. Shockey, Pullman, WA (US); Judy Schnurr, Pullman, WA (US); John A. Browse, Pullman, WA (US)

Correspondence Address: MEDLEN & CARROLL, LLP, 101 HOWARD STREET, SUITE 350, SAN FRANCISCO, CA, 94105, US

(73) Assignee: Washington State University Research Foundation, Pullman, WA (US)

(21) Appl. No.: 09/906,419

(22) Filed: Jul. 16, 2001

Related U.S. Application Data
(60) Provisional application No. 60/220,474, filed on Jul. 21, 2000.

(51) Int. Cl. A01H 5/00; C07H 21/04; C12N 15/82

(52) U.S. Cl. 800/278; 435/320.1; 536/23.2

(57) ABSTRACT

The present invention relates to genes encoding plant acyl-CoA synthetases and methods of their use. In particular, the present invention is related to plant acyl-coenzyme A synthetases. The present invention encompasses both native and recombinant wild-type forms of the enzymes, as well as mutant and variant forms, some of which possess altered characteristics relative to the wild-type enzyme. The present invention also relates to methods of using acyl-CoA synthetases, including altered expression in transgenic plants and expression in prokaryotes and cell culture systems.

This application claims priority from provisional application U.S. Ser. No. 60/220,474 filed on Jul. 21, 2000.

### FIELD OF THE INVENTION

The present invention relates to genes and proteins encoding plant acyl-CoA synthetases and methods of their use.

### BACKGROUND

Plant metabolism has evolved the ability to produce a diverse range of structures, including more than 20,000 different terpenoids, flavonoids, alkaloids, and fatty acids. Fatty acids have been extensively exploited for industrial uses in products such as lubricants, plasticizers, and surfactants. In fact, approximately one-third of vegetable oils produced in the world are already used for non-food purposes (Ohlrogge, J (1994) Plant Physiol. 104:821-26).

In 1999, approximately 40 million hectares of transgenic crops were planted worldwide. Included in this figure is approximately 50% of the soybean acreage in the United States, over 70% of the Canola acreage in Canada, about 20% of the United States corn crop, and about 33% of the United States cotton crop (Ohlrogge, J (1999) Curr. Opin. Plant Biol. 2:121-22).

Various laboratories around the world have attempted to modify triacylglycerol (TAG) content in oilseed crops by manipulating the genes involved in TAG biosynthesis. The TAG biosynthetic pathway involves many enzymatic reactions. An increasing number of the genes that encode these enzymes have been cloned and studied in detail with respect to the quantitative and qualitative contributions they make to the TAG composition of a particular oilseed. There are still several genes in the TAG pathway, however, that have not been cloned and characterized in detail.

Most of the efforts to modify TAG content have focused on either increasing the nutritional characteristics and chemical stability of edible oils or on introducing new and unusual fatty acids into TAGs for use in various industrial applications. Progress has been achieved through over-expression and/or suppression of a modestly small number of genes in the TAG synthesis pathway. However, to date, the alterations in fatty acid content have not been substantial enough to create truly meaningful new oilseed lines.

Thus, there remains a need to identify and characterize additional genes in the TAG synthesis pathway, the manipulation of which can contribute to altered or increased fatty acid content in oilseeds.

### SUMMARY OF THE INVENTION

The present invention relates to genes encoding plant acyl-CoA synthetases (ACS) and methods of their use. The present invention is not limited to any particular nucleic acid or amino acid sequence.

Accordingly, in some embodiments, the present invention provides compositions comprising an isolated nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11. The present invention is not limited to the nucleic acid sequences encoded by SEQ ID NOs:1-11. Indeed, it is contemplated that the present invention encompasses homologs, variants, and portions or fragments of the nucleic acids encoded by SEQ ID NOs:1-11. Accordingly, in some embodiments the present invention comprises sequences that hybridize to the nucleic acids encoded by SEQ ID NOs:1-11 under conditions of low to high stringency. In other embodiments, the present invention comprises nucleic acid sequences that compete with or inhibit the binding of the nucleic acid sequences encoded by SEQ ID NOs:1-11 to their complements. In some preferred embodiments, the nucleic acids encode a protein with Acyl-CoA synthetase activity. In some particularly preferred embodiments, the nucleic acid sequence encodes a protein that catalyzes the esterification of a fatty acid and coenzyme A. In other particularly preferred embodiments, the nucleic acid sequence encodes a protein comprising an amino acid sequence selected from the group consisting of SEQ ID NOs:12-22.

In some embodiments of the present invention, the nucleic acids described above are operably linked to a heterologous promoter. In further embodiments, the sequences described above are contained within a vector. In still further embodiments, the vectors are within a host cell. The present invention is not limited to any particular host cell. Indeed, a variety of host cells are contemplated, including, but not limited to, prokaryotic cells, eukaryotic cells, plant tissue cells, and cells in planta.

In some embodiments, the present invention provides methods for altering the phenotype of a plant comprising: providing i) a vector comprising a nucleic acid sequence encoding a protein, said nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11; and ii) plant tissue; and transfecting the plant tissue with the vector under conditions such that the protein is expressed. In other embodiments, the nucleic acid sequence encodes a protein comprising an amino acid sequence selected from the group consisting of SEQ ID NOs:12-22. In yet other embodiments, the nucleic acid sequence is selected from the group consisting of nucleic acid sequences that hybridize to SEQ ID NOs:1-10 under low stringency conditions.

In other embodiments, the present invention provides methods for assaying acyl-CoA synthetase activity comprising: providing a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11; expressing the nucleic acid sequence under conditions such that a protein is produced; and assaying the activity of the protein. In other embodiments, the nucleic acid sequence encodes a protein comprising an amino acid sequence selected from the group consisting of SEQ ID NOs:12-22. In yet other embodiments, the nucleic acid sequence is selected from the group consisting of nucleic acid sequences that hybridize to SEQ ID NOs:1-10 under low stringency conditions.

The present invention also provides methods for altering the phenotype of a plant comprising: providing: i) a vector comprising an antisense sequence corresponding to any of the nucleic acid sequences described above; and ii) plant tissue; and b) transfecting the plant tissue with the vector under conditions such that the antisense sequence is expressed and the activity of an acyl-CoA synthetase is down regulated as compared to wild-type plants. In particularly preferred embodiments, the nucleic acid sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

The present invention also provides methods for producing variants of acyl-CoA synthetases comprising: providing any of the nucleic acid sequences described above; mutagenizing the nucleic acid sequence; and screening the variant for activity. In particularly preferred embodiments, the nucleic acid sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

The present invention also provides methods for screening acyl-CoA synthetases comprising: providing a candidate acyl-CoA synthetase; and analyzing the candidate acyl-CoA synthetase for the presence of at least one of ACS motifs 1-9.

In additional embodiments, the present invention provides nucleic acids encoding a plant acyl-CoA synthetase, wherein the plant acyl-CoA synthetase competes for binding to a fatty acid substrate with a protein encoded by a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11.

In other embodiments, the present invention provides compositions comprising purified acyl-CoA synthetases comprising amino acid sequences SEQ ID NOs: 12-22, and portions thereof. In still other embodiments, the present invention provides compositions comprising AMP-binding proteins comprising amino acid sequences SEQ ID NOs:33-42.

In some embodiments, the present invention provides compositions comprising an isolated nucleic acid sequence selected from the group consisting of SEQ ID NOs:23-32. The present invention is not limited to the nucleic acid sequences encoded by SEQ ID NOs:23-32. Indeed, it is contemplated that the present invention encompasses homologs, variants, and portions or fragments of the nucleic acids encoded by SEQ ID NOs: 23-32. Accordingly, in some embodiments, the present invention comprises sequences that hybridize to the nucleic acids encoded by SEQ ID NOs: 23-32 under conditions of low to high stringency. In other embodiments, the present invention comprises nucleic acid sequences that compete with or inhibit the binding of the nucleic acid sequences encoded by SEQ ID NOs: 23-32 to their complements. In some preferred embodiments, the nucleic acids encode a protein with AMP binding activity. In some embodiments of the present invention, the nucleic acids described above are operably linked to a heterologous promoter. In further embodiments, the sequences described above are contained within a vector. In still further embodiments, the vectors are within a host cell. The present invention is not limited to any particular host cell. Indeed, a variety of host cells are contemplated, including, but not limited to, prokaryotic cells, eukaryotic cells, plant tissue cells, and cells in planta.

In some embodiments, the present invention provides methods for altering the phenotype of a plant comprising: providing i) a vector comprising a nucleic acid sequence encoding a protein, said nucleic acid sequence selected from the group consisting of SEQ ID NOs: 23-32; and ii) plant tissue; and transfecting the plant tissue with the vector under conditions such that the protein is expressed.

### BRIEF DESCRIPTION OF THE DRAWINGS

FIGS. 1A-1D present an amino acid sequence alignment for Arabidopsis ACS and AMP-binding protein sequences.

FIG. 2 a comparison of the degree of conservation of the deduced amino acid sequences of and around the insertional elements of each ACS. The residues corresponding to the predicted borders of the insertional element are numbered and denoted with arrows. These residues were determined by comparing the sequences of the candidate ACS genes to those of the other AMP-BP genes that were identified in the original data base screen and which lacked the insertional element. For clarity, FIG. 2 displays only the first few amino acid residues that flank the upstream and downstream borders of the insertional region.

FIG. 3 provides the AtACS1A nucleic acid sequence (SEQ ID NO: 1).

FIG. 4 provides the AtACS1B nucleic acid sequence (SEQ ID NO: 2).

FIG. 5 provides the AtACS1C nucleic acid sequence (SEQ ID NO: 3).

FIG. 6 provides the AtACS2 nucleic acid sequence (SEQ ID NO: 4).

FIG. 7 provides the AtACS3A nucleic acid sequence (SEQ ID NO: 5).

FIG. 8 provides the AtACS3B nucleic acid sequence (SEQ ID NO: 6).

FIG. 9 provides the AtACS4A nucleic acid sequence (SEQ ID NO: 7).

FIG. 10 provides the AtACS4B nucleic acid sequence (SEQ ID NO: 8).

FIG. 11 provides the AtACS5 nucleic acid sequence (SEQ ID NO: 9).

FIG. 12 provides the AtACS6A nucleic acid sequence (SEQ ID NO: 10).

FIG. 13 provides the AtACS6B nucleic acid sequence (SEQ ID NO: 11).

FIG. 14 provides the AtACS1A amino acid sequence (SEQ ID NO: 12).

FIG. 15 provides the AtACS1B amino acid sequence (SEQ ID NO: 13).

FIG. 16 provides the AtACS1C amino acid sequence (SEQ ID NO: 14).

FIG. 17 provides the AtACS2 amino acid sequence (SEQ ID NO: 15).

FIG. 18 provides the AtACS3A amino acid sequence (SEQ ID NO: 16).

FIG. 19 provides the AtACS3B amino acid sequence (SEQ ID NO: 17).

FIG. 20 provides the AtACS4A amino acid sequence (SEQ ID NO: 18).

FIG. 21 provides the AtACS4B amino acid sequence (SEQ ID NO: 19).

FIG. 22 provides the AtACS5 amino acid sequence (SEQ ID NO: 20).

FIG. 23 provides the AtACS6A amino acid sequence (SEQ ID NO: 21).

FIG. 24 provides the AtACS6B amino acid sequence (SEQ ID NO: 22).

FIG. 25 provides the predicted AMP-BP1 nucleic acid sequence (SEQ ID NO: 23).

FIG. 26 provides the predicted AMP-BP2 nucleic acid sequence (SEQ ID NO: 24).

FIG. 27 provides the predicted AMP-BP3 nucleic acid sequence (SEQ ID NO: 25).

FIG. 28 provides the predicted AMP-BP4 nucleic acid sequence (SEQ ID NO: 26).

FIG. 29 provides the predicted AMP-BP5 nucleic acid sequence (SEQ ID NO: 27).

FIG. 30 provides the predicted AMP-BP6 nucleic acid sequence (SEQ ID NO: 28).

FIG. 31 provides the predicted AMP-BP7 nucleic acid sequence (SEQ ID NO: 29).

FIG. 32 provides the predicted AMP-BP8 nucleic acid sequence (SEQ ID NO: 30).

FIG. 33 provides the predicted AMP-BP9 nucleic acid sequence (SEQ ID NO: 31).

FIG. 34 provides the predicted AMP-BP10 nucleic acid sequence (SEQ ID NO: 32).

FIG. 35 provides the predicted AMP-BP1 amino acid sequence (SEQ ID NO: 33).

FIG. 36 provides the predicted AMP-BP2 amino acid sequence (SEQ ID NO: 35).

FIG. 37 provides the predicted AMP-BP3 amino acid sequence (SEQ ID NO: 35).

FIG. 38 provides the predicted AMP-BP4 amino acid sequence (SEQ ID NO: 36).

FIG. 39 provides the predicted AMP-BP5 amino acid sequence (SEQ ID NO: 37).

FIG. 40 provides the predicted AMP-BP6 amino acid sequence (SEQ ID NO: 38).

FIG. 41 provides the predicted AMP-BP7 amino acid sequence (SEQ ID NO: 39).

FIG. 42 provides the predicted AMP-BP8 amino acid sequence (SEQ ID NO: 40).

FIG. 43 provides the predicted AMP-BP9 amino acid sequence (SEQ ID NO: 41).

FIG. 44 provides the predicted AMP-BP10 amino acid sequence (SEQ ID NO: 42).

FIG. 45 is an amino acid sequence alignment for ACS motif 1 (SEQ ID NO:43).

FIG. 46 is an amino acid sequence alignment for ACS motif 2 (SEQ ID NO:44).

FIG. 47 is an amino acid sequence alignment for ACS motif 3 (SEQ ID NO:45).

FIG. 48 is an amino acid sequence alignment for ACS motif 4(SEQ ID NO:46).

FIG. 49 is an amino acid sequence alignment for ACS motif 5 (SEQ ID NO:47).

FIG. 50 is an amino acid sequence alignment for ACS motif 6 (SEQ ID NO:48).

FIG. 51 is an amino acid sequence alignment for ACS motif 7 (SEQ ID NO:49).

FIG. 52 is an amino acid sequence alignment for ACS motif 8 (SEQ ID NO:50).

FIG. 53 is an amino acid sequence alignment for ACS motif 9 (SEQ ID NO:51).

FIG. 54 shows a phylogenetic tree constructed to visually compare the relationship between each of the candidate ACS genes.

FIG. 55 shows the results of acyl-CoA synthetase activity from in vitro assays.

FIG. 56 shows the results of a fatty acid analysis of the siliques from wild-type and AtACS6B knockout mutant Arabidopsis 42 day old plants grown under 14:10 photoperiod. The total lipids were derivatized with an internal standard using 2.5% H₂SO₄ in methanol and the fatty acid methyl esters were analyzed by gas chromatography. Values are means +/−SE (n=12).

### DESCRIPTION OF THE INVENTION

The present invention relates to genes encoding plant acyl-CoA synthetases (ACSs) and methods of their use. The present invention encompasses both native and recombinant wild-type forms of the enzyme, as well as mutant and variant forms, some of which possess altered characteristics relative to the wild-type enzyme. The present invention also relates to methods of using ACSs, including altered expression in transgenic plants and expression in prokaryotes and cell culture systems. After the “Definitions,” the following description of the invention is divided into: I. Acyl-CoA Synthetases; II. Uses of Acyl-CoA Synthetase Nucleic Acids and Polypeptides; III. Identification of Other Acyl-CoA Synthetase Homologs; and IV. AMP Binding Proteins.

### Definitions

To facilitate understanding of the invention, a number of terms are defined below.

The term “plant” as used herein refers to a plurality of plant cells which are largely differentiated into a structure that is present at any stage of a plant's development. Such structures include, but are not limited to, a fruit, shoot, stem, leaf, flower petal, etc. The term “plant tissue” includes differentiated and undifferentiated tissues of plants including, but not limited to, roots, shoots, leaves, pollen, seeds, tumor tissue and various types of cells in culture (e.g., single cells, protoplasts, embryos, callus, etc.). Plant tissue may be in planta, in organ culture, tissue culture, or cell culture.

“Oil-producing species” as used herein refers to plant species which produce and store triacylglycerol in specific organs, primarily in seeds. Such species include soybean (Glycine max), rapeseed and canola (including Brassica napus and B. campestris), sunflower (Helianthus annus), cotton (Gossypium hirsutum), corn (Zea mays), cocoa (Theobroma cacao), safflower (Carthamus tinctorius), oil palm (Elaeis guineensis), coconut palm (Cocos nucifera), flax (Linum usitatissimum), castor (Ricinus communis) and peanut (Arachis hypogaea). The group also includes non-agronomic species which are useful in developing appropriate expression vectors such as tobacco, rapid cycling Brassica species, and Arabidopsis thaliana, and wild species which may be a source of unique fatty acids.

As used herein, the term “acyl-CoA synthetase (ACS)” refers to an enzymatic activity that catalyzes the formation of an acyl-CoA-fatty acid ester from a free fatty acid and coenzyme A (CoA). As used herein, the term “plastidial acyl-CoA synthetase” refers to an enzymatic activity that catalyzes the formation of an acyl-CoA-fatty acid ester from a free fatty acid and coenzyme A and that is localized to the chloroplast. As used herein, the term “plant acyl-CoA synthetase” refers to an acyl-CoA synthetase derived from a plant. The term plant acyl-CoA synthetases encompasses both acyl CoA synthetases that are identical to wild-type plant acyl-CoA synthetases and those that are derived from wild type plant acyl-CoA synthetases (e.g., variants of plant acyl CoA synthetases or chimeric genes constructed with portions of plant acyl CoA synthetase coding regions).

As used herein, the term “AMP binding protein” (“AMP-BP”) refers to a protein comprising an AMP-binding motif, which is found in all ACS genes. This motif is associated with the ability of a protein to bind ATP and to create an acyl- or acetyl-adenylate intermediate. However, not all AMP-BPs are ACSs; thus, in addition to ACS, the AMP-BP superfamily also contains several other classes of genes, at least some of which, such as 4-coumarate-CoA ligases and acetyl-CoA synthetases, are known to exist in plants.

The term “gene” as used herein, refers to a DNA sequence that comprises control and coding sequences necessary for the production of a polypeptide or protein precursor. The polypeptide can be encoded by a full length coding sequence or by any portion of the coding sequence, as long as the desired protein activity is retained.

“Nucleoside”, as used herein, refers to a compound consisting of a purine [guanine (G) or adenine (A)] or pyrimidine [thymine (T), uridine (U), or cytidine (C)] base covalently linked to a pentose, whereas “nucleotide” refers to a nucleoside phosphorylated at one of its pentose hydroxyl groups.

A “nucleic acid”, as used herein, is a covalently linked sequence of nucleotides in which the 3′ position of the pentose of one nucleotide is joined by a phosphodiester group to the 5′ position of the pentose of the next, and in which the nucleotide residues (bases) are linked in specific sequence; i.e., a linear order of nucleotides. A “polynucleotide”, as used herein, is a nucleic acid containing a sequence that is greater than about 100 nucleotides in length. An “oligonucleotide”, as used herein, is a short polynucleotide or a portion of a polynucleotide. An oligonucleotide typically contains a sequence of about two to about one hundred bases. The word “oligo” is sometimes used in place of the word “oligonucleotide”.

Nucleic acid molecules are said to have a “5′-terminus” (5′ end) and a “3′-terminus” (3′ end) because nucleic acid phosphodiester linkages occur to the 5′ carbon and 3′ carbon of the pentose ring of the substituent mononucleotides. The end of a nucleic acid at which a new linkage would be to a 5′ carbon is its 5′ terminal nucleotide. The end of a nucleic acid at which a new linkage would be to a 3′ carbon is its 3′ terminal nucleotide. A terminal nucleotide, as used herein, is the nucleotide at the end position of the 3′- or 5′-terminus.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring.

As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. Typically, promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “wild-type” when made in reference to a gene refers to a gene which has the characteristics of a gene isolated from a naturally occurring source. The term “wild-type” when made in reference to a gene product refers to a gene product which has the characteristics of a gene product isolated from a naturally occurring source. A wild-type gene is that which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “antisense” as used herein refers to a deoxyribonucleotide sequence whose sequence of deoxyribonucleotide residues is in reverse 5′ to 3′ orientation in relation to the sequence of deoxyribonucleotide residues in a sense strand of a DNA duplex. A “sense strand” of a DNA duplex refers to a strand in a DNA duplex which is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein. As used herein, the term “over-expression” refers to the production of a gene product in transgenic organisms that exceeds levels of production in normal or non-transformed organisms. As used herein, the term “cosuppression” refers to the expression of a foreign gene which has substantial homology to an endogenous gene resulting in the suppression of expression of both the foreign and the endogenous gene. As used herein, the term “altered levels” refers to the production of gene product(s) in transgenic organisms in amounts or proportions that differ from that of normal or non-transformed organisms.

The term “recombinant” when made in reference to a DNA molecule refers to a DNA molecule which is comprised of segments of DNA joined together by means of molecular biological techniques. The term “recombinant” when made in reference to a protein or a polypeptide refers to a protein molecule which is expressed using a recombinant DNA molecule.

The term “nucleotide sequence of interest” refers to any nucleotide sequence, the manipulation of which may be deemed desirable for any reason (e.g., confer improved qualities), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product, (e.g., promoter sequence, polyadenylation sequence, termination sequence, enhancer sequence, etc.).

As used herein the term “coding region” when used in reference to structural gene refers to the nucleotide sequences which encode the amino acids found in the nascent polypeptide as a result of translation of a mRNA molecule. Typically, the coding region is bounded on the 5′ side by the nucleotide triplet “ATG” which encodes the initiator methionine and on the 3′ side by a stop codon (e.g., TAA, TAG, TGA). In some cases the coding region is also known to initiate by a nucleotide triplet “TTG”.

As used herein, the terms “complementary” or “complementarity” when used in reference to polynucleotides refer to polynucleotides which are related by the base-pairing rules. For example, for the sequence 5′-AGT-3′ is complementary to the sequence 5′-ACT-3′. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

A “complement” of a nucleic acid sequence as used herein refers to a nucleotide sequence whose nucleic acids show total complementarity to the nucleic acids of the nucleic acid sequence.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence which is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe which can hybridize to either or both strands of the double-stranded nucleic acid sequence under conditions of low stringency as described infra.

Low stringency conditions when used in reference to nucleic acid hybridization comprise conditions equivalent to binding or hybridization at 42_C in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharmacia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

High stringency conditions when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above listed conditions.

“Stringency” when used in reference to nucleic acid hybridization typically occurs in a range from about Tₘ-5_C (5_C below the Tₘ of the probe) to about 20_C to 25_C below Tₘ. As will be understood by those of skill in the art, a stringent hybridization can be used to identify or detect identical polynucleotide sequences or to identify or detect similar or related polynucleotide sequences. Under “stringent conditions” a nucleic acid sequence of interest will hybridize to its exact complement and closely related sequences.

Polypeptide molecules are said to have an “amino terminus” (N-terminus) and a “carboxy terminus” (C-terminus) because peptide linkages occur between the backbone amino group of a first amino acid residue and the backbone carboxyl group of a second amino acid residue. Typically, the terminus of a polypeptide at which a new linkage would be to the carboxy-terminus of the growing polypeptide chain, and polypeptide sequences are written from left to right beginning at the amino terminus.

As used herein in reference to an amino acid sequence or a protein, the term “portion” (as in “a portion of an amino acid sequence”) refers to fragments of that protein. The fragments may range in size from four amino acid residues to the entire amino acid sequence minus one amino acid.

As used herein, the term “fusion protein” refers to a chimeric protein containing the protein of interest (e.g., ACSs and fragments thereof) joined to an exogenous protein fragment (e.g., the fusion partner which consists of a non-ACS protein). The fusion partner may enhance the solubility of ACS protein as expressed in a host cell, may provide an affinity tag to allow purification of the recombinant fusion protein from the host cell or culture supernatant, or both. If desired, the fusion protein may be removed from the protein of interest (e.g., ACS or fragments thereof) by a variety of enzymatic or chemical means know to the art.

As used herein, the term “transit peptide” refers to the N-terminal extension of a protein that serves as a signal for uptake and transport of that protein into an organelle such as a plastid or mitochondrion.

The term “isolated” when used in relation to a nucleic acid, as in “an isolated nucleic acid sequence” refers to a nucleic acid sequence that is identified and separated from at least one contaminant nucleic acid with which it is ordinarily associated in its natural source. Isolated nucleic acid is nucleic acid present in a form or setting that is different from that in which it is found in nature. In contrast, non-isolated nucleic acids are nucleic acids such as DNA and RNA which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, an isolated nucleic acid sequence comprising SEQ ID NO:1 includes, by way of example, such nucleic acid sequences in cells which ordinarily contain SEQ ID NO:1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

As used herein, the term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the terms “vector” and “vehicle” are used interchangeably in reference to nucleic acid molecules that transfer DNA segment(s) from one cell to another. Vectors may include plasmids, bacteriophages, viruses, cosmids, and the like.

The term “expression vector” or “expression cassette” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “targeting vector” or “targeting construct” refer to oligonucleotide sequences comprising a gene of interest flanked on either side by a recognition sequence which is capable of homologous recombination of the DNA sequence located between the flanking recognition sequences.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “selectable marker” as used herein, refer to a gene which encodes an enzyme having an activity that confers resistance to an antibiotic or drug upon the cell in which the selectable marker is expressed. Selectable markers may be “positive” or “negative.” Examples of positive selectable markers include the neomycin phosphotrasferase (NPTII) gene which confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Promoters may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue (e.g., leaves). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody which is specific for the polypeptide product encoded by the nucleotide sequence of interest whose expression is controlled by the promoter. A labeled (e.g., peroxidase conjugated) secondary antibody which is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. Exemplary constitutive plant promoters include, but are not limited to SD Cauliflower Mosaic Virus (CaMV SD; see e.g., U.S. Pat. No. 5,352,605, incorporated herein by reference), mannopine synthase, octopine synthase (ocs), superpromoter (see e.g., WO 95/14098), and ubi3 (see e.g., Garbarino and Belknap (1994) Plant Mol. Biol. 24:119-127) promoters. Such promoters have been used successfully to direct the expression of heterologous nucleic acid sequences in transformed plant tissue.

In contrast, a “regulatable” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

As used herein, the term “regulatory element” refers to a genetic element that controls some aspect of the expression of nucleic acid sequence(s). For example, a promoter is a regulatory element that facilitates the initiation of transcription of an operably linked coding region. Other regulatory elements are splicing signals, polyadenylation signals, termination signals, etc.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al. (1989) Molecular Cloning: A Laboratory Manual, 2nd ed. (Cold Spring Harbor Laboratory Press, New York) pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHI/BclI restriction fragment and directs both termination and polyadenylation (Sambrook, supra, at 16.6-16.7).

The terms “infecting” and “infection” with a bacterium refer to co-incubation of a target biological sample, (e.g., cell, tissue, etc.) with the bacterium under conditions such that nucleic acid sequences contained within the bacterium are introduced into one or more cells of the target biological sample.

The term “Agrobacterium” refers to a soil-borne, Gram-negative, rod-shaped phytopathogenic bacterium which causes crown gall. The term “Agrobacterium” includes, but is not limited to, the strains Agrobacterium tumefaciens, (which typically causes crown gall in infected plants), and Agrobacterium rhizogens (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain LBA4301, C58, A208, GV3101) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are incorporated herein by reference), and are commercially available (e.g., the helium gas-driven microprojectile accelerator (PDS-1000/He, BioRad).

The term “microwounding” when made in reference to plant tissue refers to the introduction of microscopic wounds in that tissue. Microwounding may be achieved by, for example, particle bombardment as described herein.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “transgenic” when used in reference to a cell refers to a cell which contains a transgene, or whose genome has been altered by the introduction of a transgene. The term “transgenic” when used in reference to a tissue or to a plant refers to a tissue or plant, respectively, which comprises one or more cells that contain a transgene, or whose genome has been altered by the introduction of a transgene. Transgenic cells, tissues and plants may be produced by several methods including the introduction of a “transgene” comprising nucleic acid (usually DNA) into a target cell or integration of the transgene into a chromosome of a target cell by way of human intervention, such as by the methods described herein.

The term “transgene” as used herein refers to any nucleic acid sequence which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence which is naturally found in the cell into which it is introduced so long as it does not contain some modification (e.g., a point mutation, the presence of a selectable marker gene, etc.) relative to the naturally-occurring sequence. The term “heterologous DNA sequence” refers to a nucleotide sequence which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Heterologous DNA also includes an endogenous DNA sequence which contains some modification. Generally, although not necessarily, heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

The term “foreign gene” refers to any nucleic acid (e.g., gene sequence) which is introduced into the genome of a cell by experimental manipulations and may include gene sequences found in that cell so long as the introduced gene contains some modification (e.g., a point mutation, the presence of a selectable marker gene, etc.) relative to the naturally-occurring gene.

The term “transformation” as used herein refers to the introduction of a transgene into a cell. Transformation of a cell may be stable or transient. The term “transient transformation” or “transiently transformed” refers to the introduction of one or more transgenes into a cell in the absence of integration of the transgene into the host cell's genome. Transient transformation may be detected by, for example, enzyme-linked immunosorbent assay (ELISA) which detects the presence of a polypeptide encoded by one or more of the transgenes. Alternatively, transient transformation may be detected by detecting the activity of the protein (e.g., _-glucuronidase) encoded by the transgene. The term “transient transformant” refers to a cell which has transiently incorporated one or more transgenes. In contrast, the term “stable transformation” or “stably transformed” refers to the introduction and integration of one or more transgenes into the genome of a cell. Stable transformation of a cell may be detected by Southern blot hybridization of genomic DNA of the cell with nucleic acid sequences which are capable of binding to one or more of the transgenes. Alternatively, stable transformation of a cell may also be detected by the polymerase chain reaction of genomic DNA of the cell to amplify transgene sequences. The term “stable transformant” refers to a cell which has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene.

The term “amplification” is defined as the production of additional copies of a nucleic acid sequence and is generally carried out using polymerase chain reaction technologies well known in the art (Dieffenbach and G S Dvekler, (1995) PCR Primer, a Laboratory Manual, Cold Spring Harbor Press, Plainview N.Y.). As used herein, the term “polymerase chain reaction” (“PCR”) refers to the methods disclosed in U.S. Pat. Nos. 4,683,195, 4,683,202 and 4,965,188, all of which are incorporated herein by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; and/or incorporation of ³²P-labeled deoxyribonucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications. Amplified target sequences may be used to obtain segments of DNA (e.g., genes) for the construction of targeting vectors, transgenes, etc.

As used herein, the term “sample template” refers to a nucleic acid originating from a sample which is analyzed for the presence of “target”. In contrast, “background template” is used in reference to nucleic acid other than sample template, which may or may not be present in a sample. Background template is most often inadvertent. It may be the result of carryover, or it may be due to the presence of nucleic acid contaminants sought to be purified away from the sample. For example, nucleic acids other than those to be detected may be present as background in a test sample.

As used herein, the term “primer” refers to an oligonucleotide, whether occurring naturally (e.g., as in a purified restriction digest) or produced synthetically, which is capable of acting as a point of initiation of nucleic acid synthesis when placed under conditions in which synthesis of a primer extension product which is complementary to a nucleic acid strand is induced (i.e., in the presence of nucleotides, an inducing agent such as DNA polymerase, and under suitable conditions of temperature and pH). The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and use of the method.

As used herein, the term “probe” refers to an oligonucleotide (i.e., a sequence of nucleotides), whether occurring naturally (e.g., as in a purified restriction digest) or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that the probe used in the present invention is labeled with any “reporter molecule,” so that it is detectable in a detection system, including, but not limited to enzyme (i.e., ELISA, as well as enzyme-based histochemical assays), fluorescent, radioactive, and luminescent systems. It is not intended that the present invention be limited to any particular detection system or label. The terms “reporter molecule” and “label” are used herein interchangeably. In addition to probes, primers and deoxynucleoside triphosphates may contain labels; these labels may comprise, but are not limited to, ³²P, ³³P, ^{SD}, enzymes, or fluorescent molecules (e.g., fluorescent dyes).

I. Acyl-CoA Synthetases

Acyl-CoA synthetases (ACSs) catalyze the following reaction:

Fatty acid+CoASH+ATP! acyl-CoA+AMP+PPi

wherein free fatty acids are activated through ATP-dependent thioesterification to coenzyme A. This reaction is critical to most fatty acid metabolism, since all but a few fatty acid-utilizing enzymes require activated forms of these molecules as substrates. The ACSs are particularly important to plant fatty acid metabolism. The present invention is not limited to any particular mechanism. Indeed, an understanding of the mechanism is not required to practice the present invention. However, it is contemplated that free fatty acids synthesized in the chloroplasts undergo activation by ACS at the plastid outer envelope membrane before being incorporated into TAG in the endoplasmic reticulum. Therefore, modifications of fatty acid distribution in TAG pools within a seed are likely affected by the various isoforms of ACS.

In addition to their roles in TAG biosynthesis, ACSs are thought to perform other important functions within the plant cell. It is contemplated that altered expression of the ACSs of the present invention may be utilized to alter these functions. For example, ACS is necessary for activating fatty acids released from oil bodies in newly germinated seedlings. These acyl-CoAs serve as substrates for the beta-oxidation cycle, which supplies the plant with cellular energy until it becomes photosynthetically competent. ACS may also play a role in cuticle wax synthesis. The cuticle waxes are a mixture of hydrophobic lipid compounds found on the surfaces of the aerial tissues of most plants. These waxes retard water loss, protect the plants from pests, and provide signaling molecules needed for fertility.

ACS is also a necessary component of the process of protein acylation. Several essential proteins and enzymes characterized in other eukaryotic organisms undergo coupling between myristic and/or palmitic acids and specific amino acid residues near their N-termini. These fatty acid modifications are necessary for proper targeting and function of these proteins. Most of the acylated target proteins are involved in signal transduction or metabolic regulation. The fatty acids used for these modifications must be supplied as acyl-CoAs.

ACS also catalyzes the first step in the biosynthetic pathway of biotin, a vitamin cofactor necessary for many carboxylation/decarboxylation reactions. ACS may also play an important role in the synthesis of jasmonic acid, an important fatty acid-derived signaling compound involved in reproduction, plant defense, and a number of other plant response reactions.

One of the major goals of modern plant biotechnology is to manipulate lipid metabolism in oilseed crops to produce new and improved edible and industrial vegetable oils. Lipids constitute the structural components of cellular membranes and act as sources of energy for the germinating seed. Both de novo synthesis and modification of existing lipids are dependent on the activity of ACSs, as described above. To date, ACSs have been recalcitrant to traditional methods of purification due to their association with membranes.

Despite their crucial role in lipid metabolism, ACSs have not been well-characterized in plants. To date, the only molecular information regarding plant ACSs is provided by Fulda et al., Plant Molec. Biol. 33:911-22 (1997), who describe five cDNA clones from Brassica napus, only two of which had ACS activity when expressed in E. coli. The present inventors have identified and cloned over 20 different genes, eleven of which are identified as ACSs; the remaining genes are AMP-BPs. These results indicate that, surprisingly, ACS exists as a much larger gene family in plants than could have been predicted from the results of Fulda et al.

The ACS genes were discovered by a step-wise procedure. The first step was computer-assisted homology comparisons between amino acid sequences of known eukaryotic ACS sequences and EST sequences of Arabidopsis genome databases. Potential candidates, or ACS homologs, were then screened for the presence of a unique 40-50 position amino acid insertion near the middle of proteins encoded by ACS genes from Bassica napus; the results identified eleven genes as encoding ACSs. The sequences of the ACS genes were then compared by GAP analysis to establish that each gene was unique. The results of this analysis were also utilized to determine the relationships between the different genes; these relationships formed the basis on which to name the genes. The ACS homologs were also screened for activity by functional expression in Saccharomyces cerevisiae YB525 and for in vitro activity. Additional information about the identity and role of the ACS genes was obtained from analysis of their tissue-specific expression pattern, and chloroplast import assays. Furthermore, T-DNA Arabidopsis mutants lacking an ACS gene have been identified and are described.

Eleven ACS genes have been identified. This family therefore represents the largest ACS gene family yet described in a single species, surpassing even that of humans, which family is known to contain at least six genes that encode ACS or VLCS (very long chain acyl-CoA synthetase) ((Steinberg, S. J et al. (2000) Journal of Biological Chemistry 275(45): 35162-35169).

Accordingly, the present invention describes the isolation of several isoforms of ACS genes from Arabidopsis thaliana. It is contemplated that these genes and their homologs and variants will find use in the development of plants containing specialized fatty acid compositions. Each of these genes is discussed in further detail below.

A. ACS Nucleic Acids

Nucleic acids encoding plant ACSs were identified in the following manner. BLAST searches of the Arabidopsis genome database were conducted for EST sequences encoding polypeptides having homology to amino acid sequences of E. coli, rat, and yeast ACSs. ESTs having homology to the ACS genes were then ordered from the Arabidopsis Biological Resource Center (ABRC, Ohio State University) and used to screen a 2-3 kb size selected library (also from the ABRC). Full-length cDNAs were cloned into pPCR-Script Cam vectors (Stratagene) or pYES2 vectors (Stratagene) and sequenced.

Computer-assisted homology comparisons between known eukaryotic ACS sequences and the Arabidopsis sequences found either in library screens or in the public databases revealed more than 40 genes containing significant homology to known ACSs from other eukaryotic organisms. Each of these genes contained the AMP-binding protein signature motif, which is found in all ACS genes; therefore, these genes were considered “ACS homologs.” However, the identification of ACS genes from this simple sequence analysis was not possible. This is because other groups of proteins also contain the AMP-binding protein signature motif; thus, while all ACSs are AMP-binding proteins, the reverse is not true. In addition to ACS, the AMP-BP superfamily also contains several other classes of genes, some of which, such as 4-coumarate-CoA ligases and acetyl-CoA synthetases, are known to exist in plants. Therefore, what was needed was a more definitive ACS-specific sequence determinate with which to identify more likely ACS candidate genes.

Previous studies identified a unique 40-50 amino acid insertion near the middle of ACS enzymes in Brassica napus ((Fulda, M et al. (1997) Plant Mol Biol 33(5): 911-22) and rat (Iijima, H. et al. (1996) Eur J Biochem 242(2): 186-90). Although the precise function of the insertion was unknown, evidence indicated that it might be a necessary component of eukaryotic ACS gene function. Moreover, both the length and the location of this insertion is quite closely conserved between the rapeseed and rat clones, spanning approximately amino acid residues 330 to 380 within proteins of about 660 amino acids total. This sequence insertion was also found in many other eukaryotic ACSs known to activate long-chain (C14-C20) fatty acids (Fujino and Yamamoto, 1992), (Johnson, D R et al. (1994) J Cell Biol 127(3): 751-62), (Kang, M J et al. (1997) Proc Natl Acad Sci U S A 94(7): 2880-4), but it was not found in the VLCS genes (very long chain fatty acyl-CoA synthetases, acyl chains>C22) (Uchiyama, A et al. (1996) J Biol Chem 271(48): 30360-5), (Berger, J et al. (1998) FEBS Lett 425(2): 305-9), (Min, K T and Benzer, S (1999) Science 284(5422): 1985-8), (Choi, J Y and Martin, C E (1999) J Biol Chem 274(8): 4671-83), (Steinberg, S J et al. (1999) Biochem and Biophys Res Comm 257(2): 615-621). It was also not found in any of the acetyl-CoA synthetases ((Ke et al., 2000)) or 4-coumarate-CoA ligases ((Lee, M et al. (1995) Science 280(5365): 915-918), (Ehlting, J et al. (1999) Plant J 19(1): 9-20) that had been cloned from Arabidopsis (data not shown). The maintenance of this sequence element in ACS genes from such evolutionarily distant species as Brassica napus and Rattus norvegicus, combined with its absence in genes that encode enzymes with specificity for short, or very long, but not long chain, fatty acids, suggested that this sequence element might be very useful as a long chain ACS-specific sequence “probe”.

Therefore, the presence of this sequence element was used as a probe to analyze the entire set of Arabidopsis genes that contained the AMP-BP signature motif. Eleven of the forty uncharacterized genes, or ACS homologs, contained insertions near the predicted sites within the deduced amino acid sequences. These eleven genes were therefore tentatively identified as ACS genes.

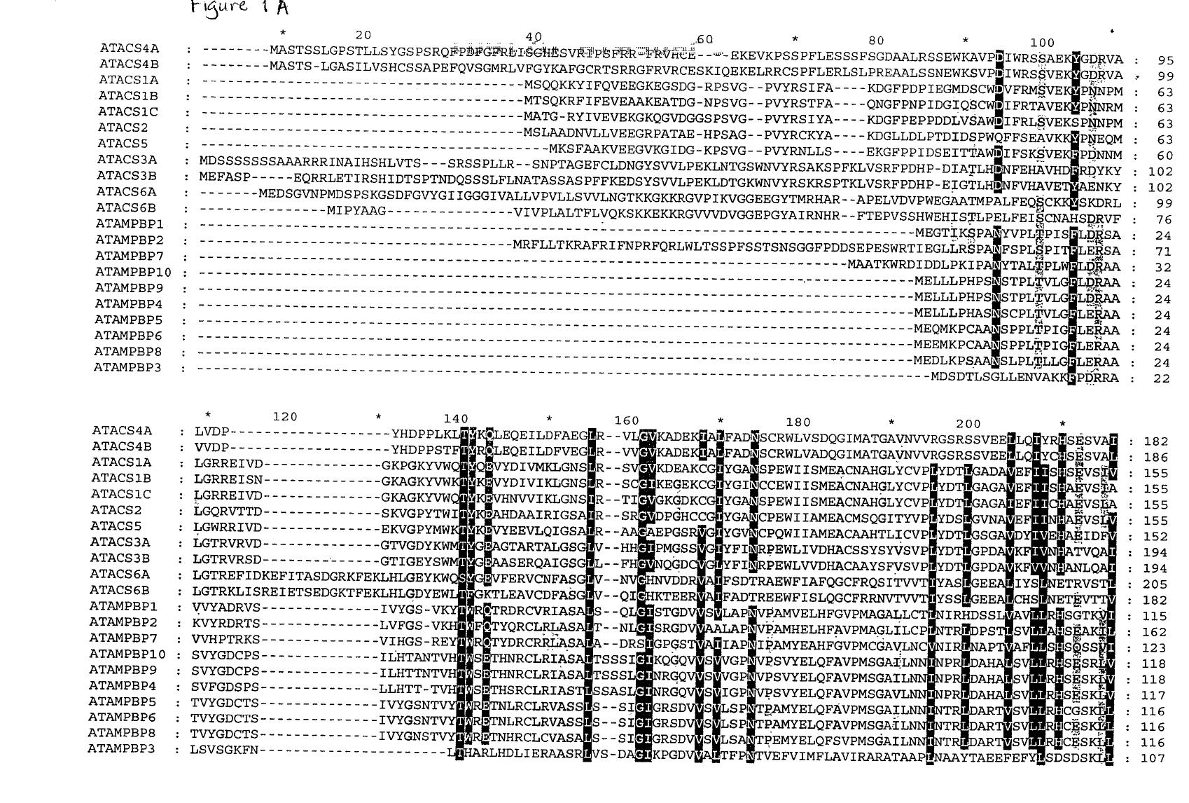

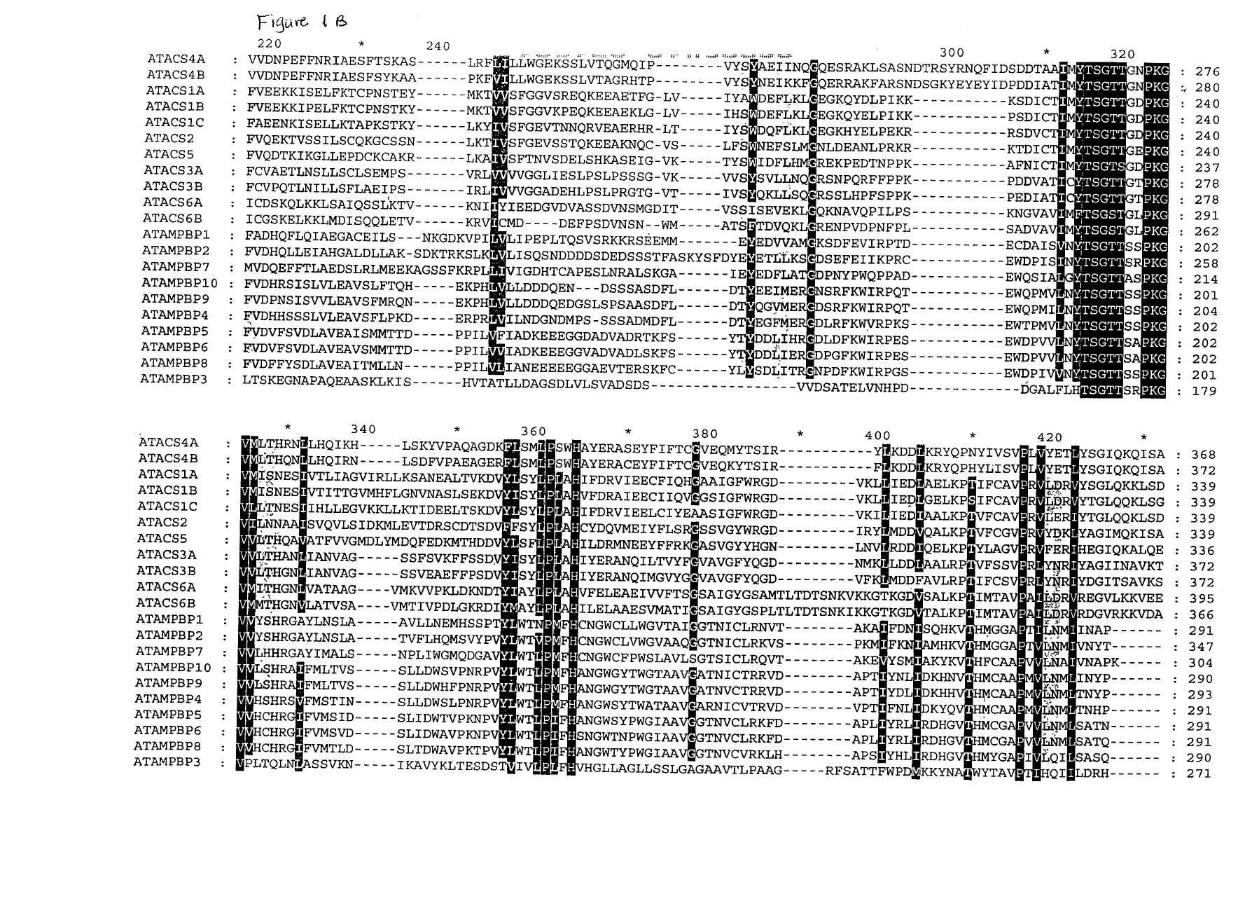

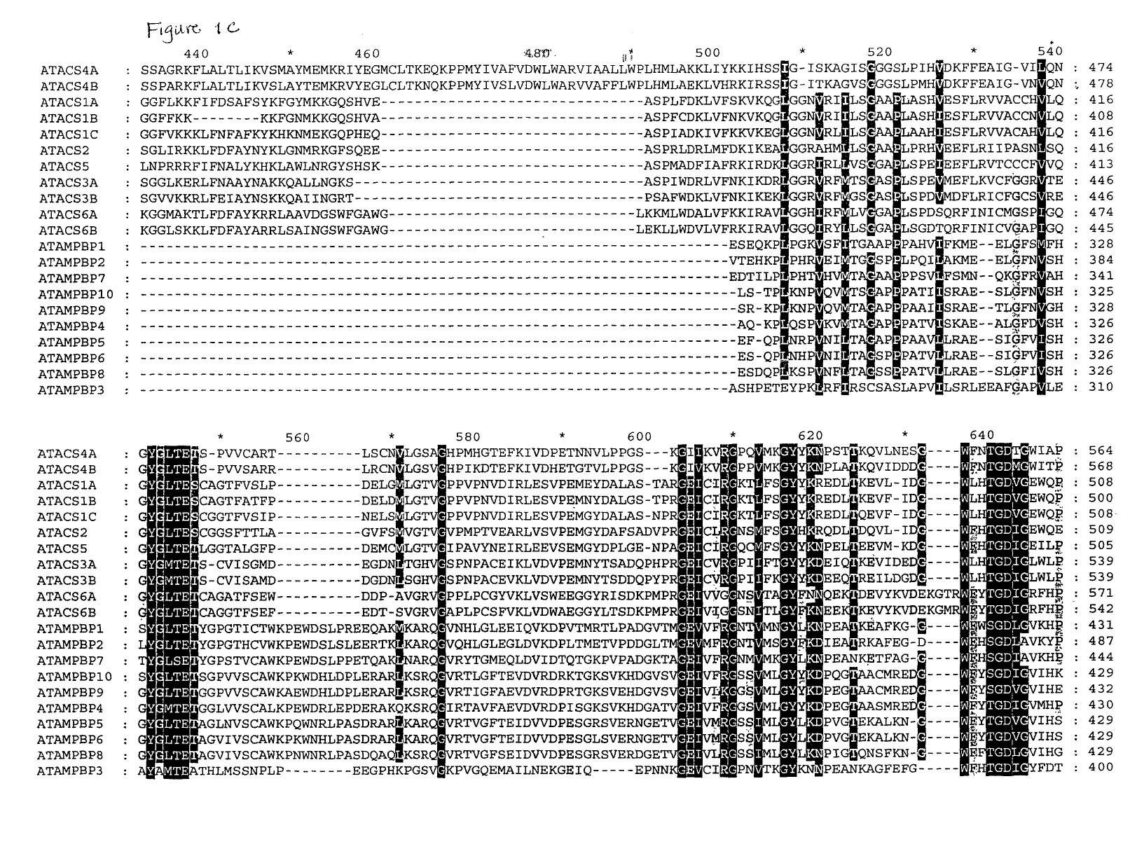

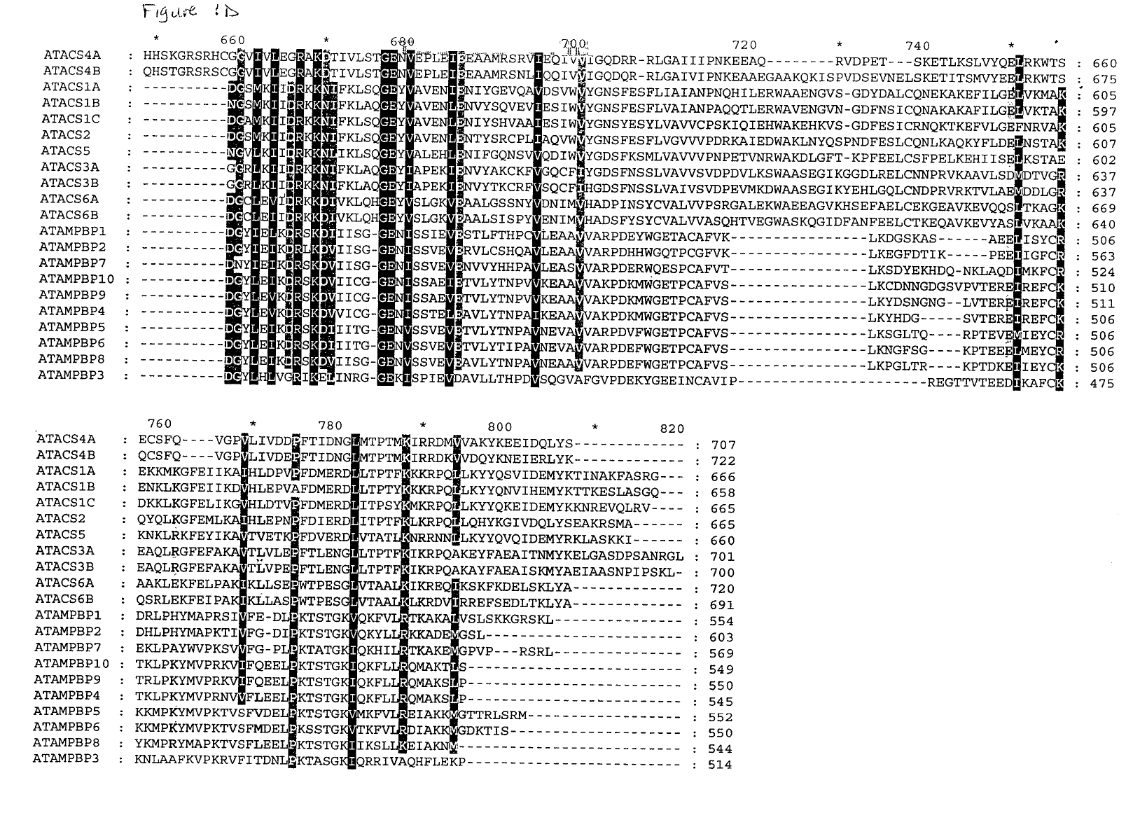

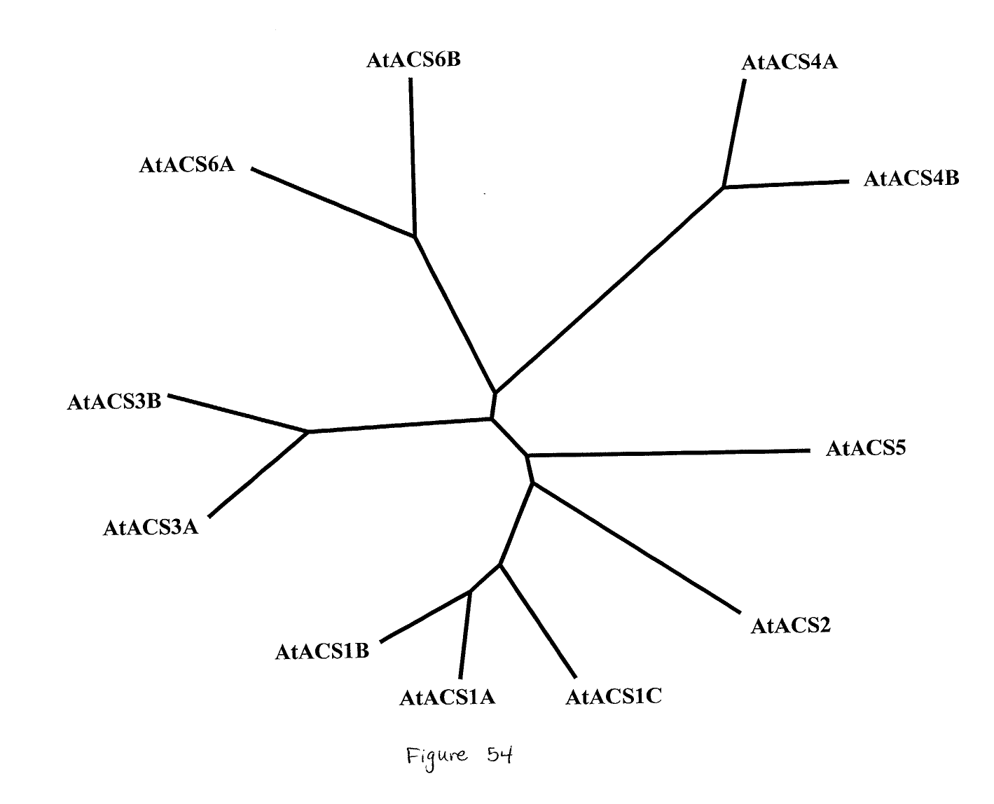

The amino acid sequences of these genes were then compared by GAP analysis; the results (as shown in FIG. 1) established that each gene was unique. The results were also used as the basis for naming these genes. The genes are named AtACS for Arabidopsis thaliana acyl-CoA synthetase. The genes are numbered starting with the number 1. If a gene possesses greater than 66% amino acid identity to any other gene(s), the number is maintained between the genes and each is lettered progressively (1A, 1B, 1C etc.). A phylogenetic tree was constructed to visually compare the relationship between each of the candidate ACS genes. This tree is shown in FIG. 54. A summary of the information pertaining to each of the AtACS genes, including the corresponding EST sequences, is shown in Table 1.

| | Genbank | Chromosome/Genomic | Corresponding ESTs |
|---|---|---|---|
| Gene Name | Accession # | clone/MIPS protein entry | (Geneback Accession #s) |
| AtACS1A | | Chromosome 4 | AV564087, AV554986, N38362, T45466, |
| | | BAC clone T32A16 | AA597813, N65639, T20845 |
| | | At4g23850 | |
| AtACS1B | | Chromosome 4 | * |
| | | BAC clone T22B4 | |
| | | At4g11030 | |
| AtACS1C | | Chromosome 1 | AI992650, AI999263, AV536372, T43231, |
| | | BAC clone F15H21 | AA395246, H77181, H76835 |
| | | At1g64400 | |
| AtACS2 | | Chromosome 1 | AV524574, AV527146, AV563196, AV518034, |
| | | BAC clone F13F21 | AV542593, AV560461, AV522512, N65171, |
| | | At1g49430 | AV520714, AV558696, AV559865, AV527730, |
| | | | BE526116, AV531977, AV521092 |
| AtACS3A | | Chromosome 3 | AV551395, AV563566, H76931 |
| | | BAC clone F2O10 | |
| | | At3g05970 | |
| AtACS3B | | Chromosome 5 | AV548579, AI994483, AA586273, T20754, |
| | | BAC clone F15A18 | T44244, BG459477, BG459383 |
| | | At5g227600 | |
| AtACS4A | | Chromosome 4 | AI999282 |
| | | BAC clone ATFCA0 | |
| | | At4g14070 | |
| AtACS4B | | Chromosome 3 | * |
| | | BAC clone MYM9 | |
| | | At3g23790 | |
| AtACS5 | | Chromosome 2 | AV559619, AV565921, A1995760, AV563860, |
| | | BAC clone T8I3 | AV560369, AV558313, AV563291, BE522084, |
| | | At2g47240 | AV556901, AV538317, AV550568, BE529524, |
| | | | AV529145, Z26001, BE522229, BE525438, |
| | | | BE524235, BE529120, BE530866, BE530784 |
| AtACS6A | | Chromosome 2 | AV526744, AV552610, N96529, T13791 |
| | | BAC clone T1O3 | |
| | | At2g04350 | |
| AtAGS6B | | Chromosome 1 | AI992417, AV556982, AV539306, AV541829, |
| | | BAC clone T5M16 | BE525296, AV567096, H76796, AV551722, |
| | | At1g77590 | H76865, BE522855 |

The ACS genes were isolated generally as follows:

AtAMP-BP3 (SEQ ID NO: 25), AtACS3A (SEQ ID NO: 5), and AtACS 6A (SEQ ID NO: 10) were isolated from the library based on homology to ESTs FAFM13, 205M6T7, and G2B10T7, respectively.

cDNAs corresponding to AtACS2 (SEQ ID NO: 4), AtACS6b (SEQ ID NO: 11), AtACS5 (SEQ ID NO: 9) were cloned from the library based on homology to ESTs 229E14T7, 203J11T7, and GbGe115a, respectively. The 5′ ends of the cDNAs were not present in the isolated clones and were cloned by 5′ RACE amplifications with total phage DNA isolated from the cDNA library.

cDNAs corresponding to AtACS3B (SEQ ID NO:6), AtACS1A (SEQ ID NO:1), and AtACS1C (SEQ ID NO: 3) were cloned from the genomic library based on homology to ESTs 123N12T7, 240K22T7, and 119E14T7, respectively. Full length cDNAs were amplified using primers designed from the genomic sequences. Corresponding cDNA clones were apparently not present in the cDNA library.

AtACS1B (SEQ ID NO:2) was identified by a BLAST search from the Arabidopsis Genome Initiative database as a homologous sequence to AtACS 1A and 1C. Primers designed to the putative start and stop codons amplify an appropriately sized product from genomic DNA but do not amplify a cDNA clone when utilized for RT-PCR. The amplified clone was longer than the predicted cDNA.

AtACS4A (SEQ ID NO:7), which was originally named AMP-BP3 and later correctly identified as AtACS4A, was identified from the Arabidopsis databases using the sequence of the Brassica AMP-BP clone pMF28P (Genbank Accession # Z72151).

AtACS4B (SEQ ID NO:8) was found in the Arabidopsis database by homology to AtACS4A.

As described above, ACSs bear strong homology to other AMP-binding proteins. Therefore, it was necessary to screen candidate ACSs to determine if they did indeed encode ACS activity. The screens were conducted by screening for complementation of the mutant Saccharomyces cerevisiae strain YB525 (Johnson et al., (1994) J. Cell. Biol. 127:751-762), which is deficient in two ACS genes. In some cases, cDNAs originally suspected of encoding ACS activity were found not to be true ACSs (e.g., AtAMP-BP1, SEQ ID NO:23, and AtAMP-BP3, SEQ ID NO: 25).

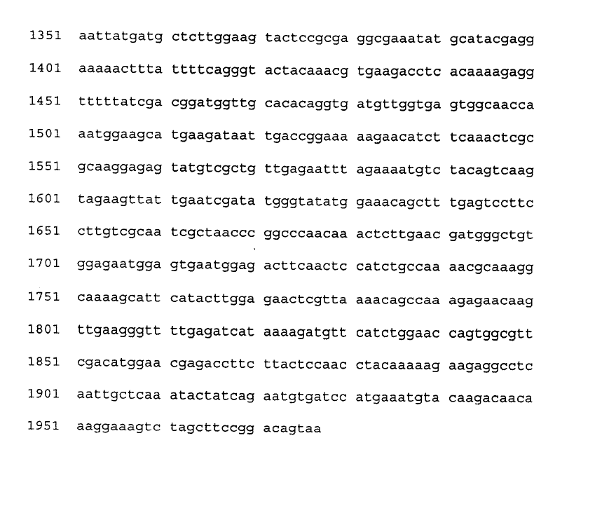

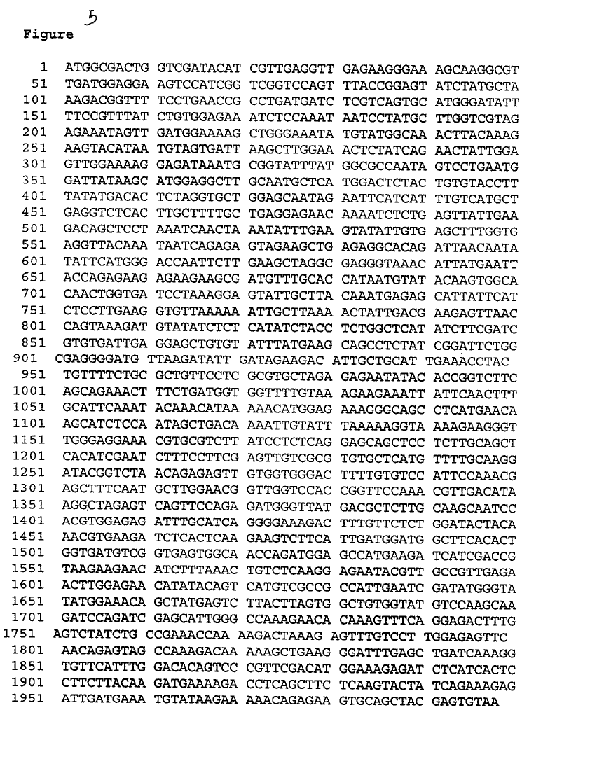

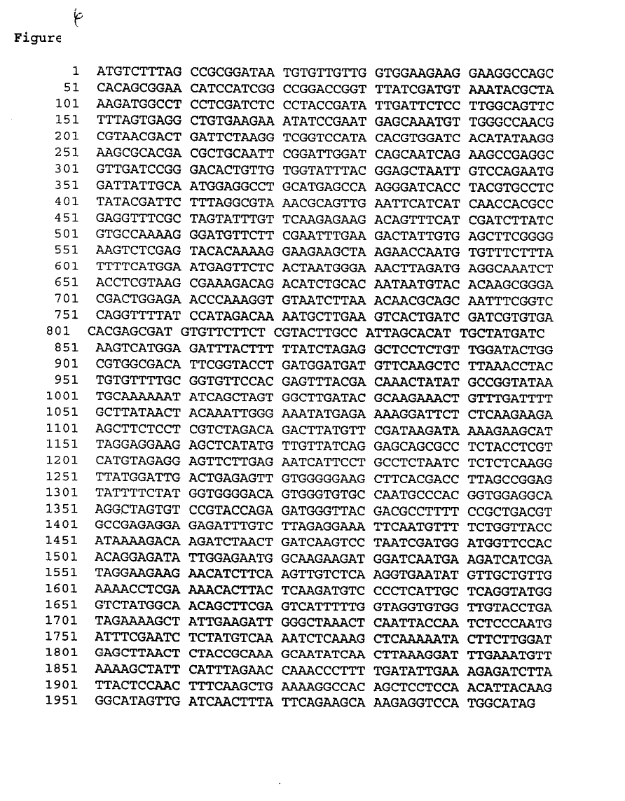

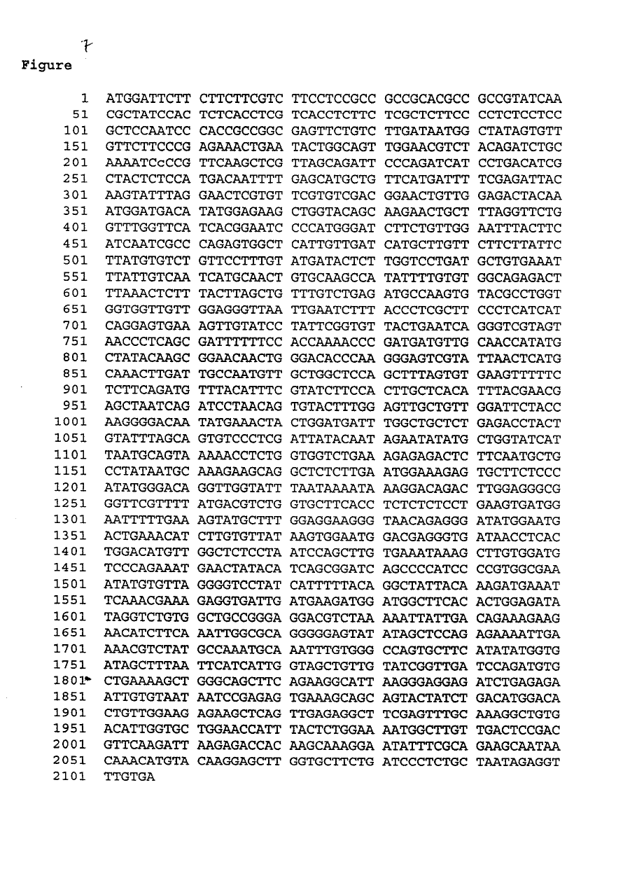

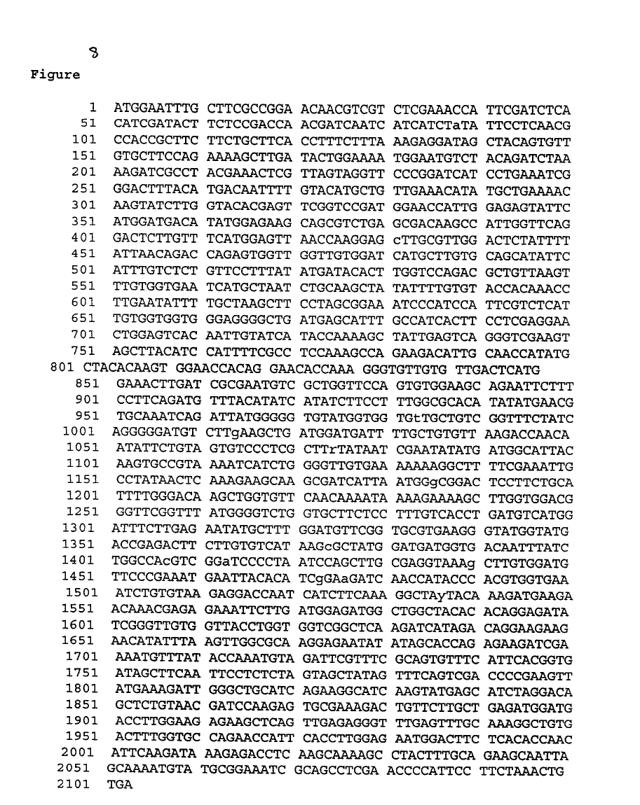

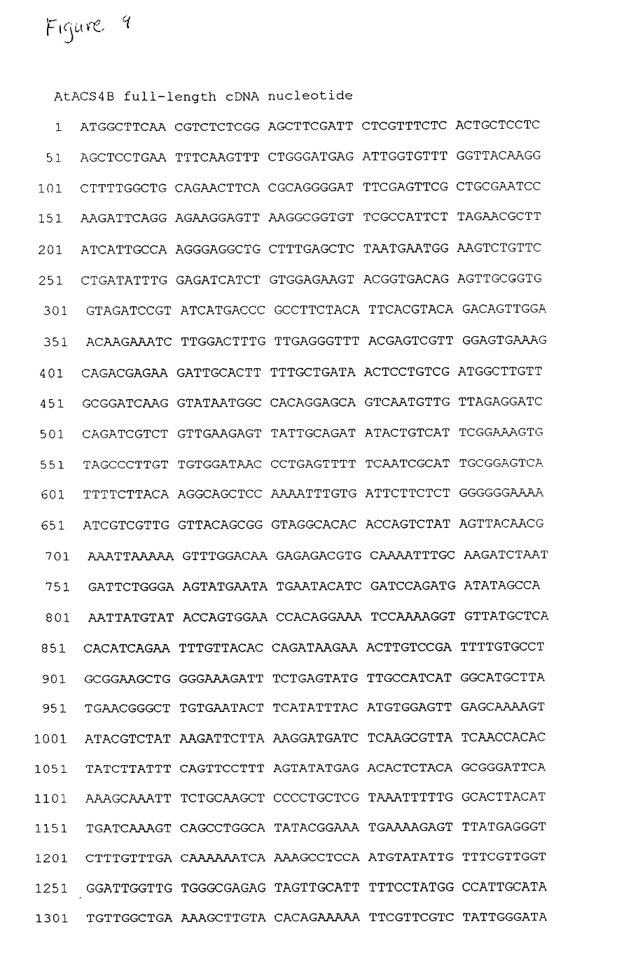

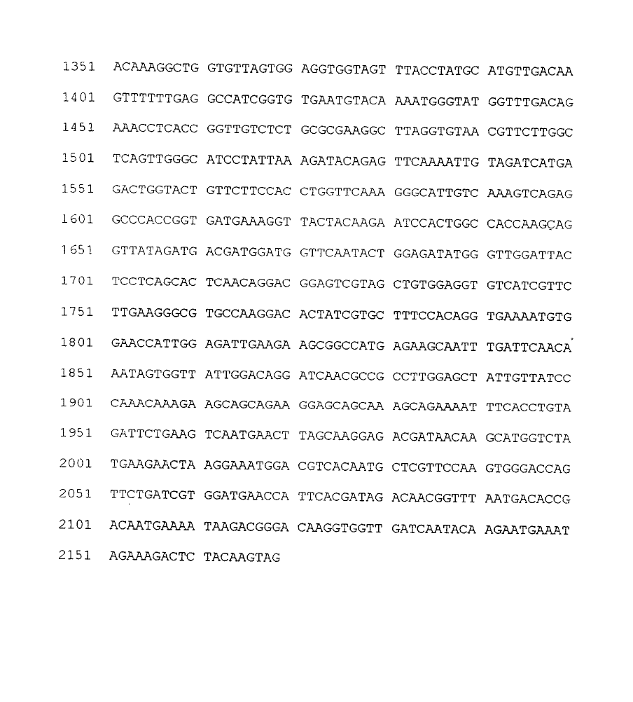

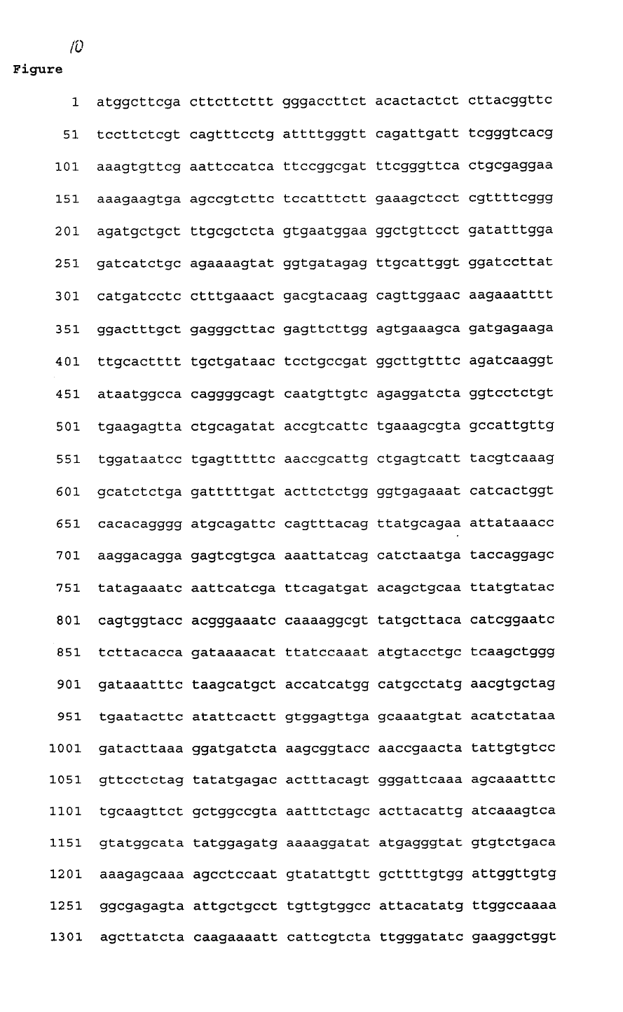

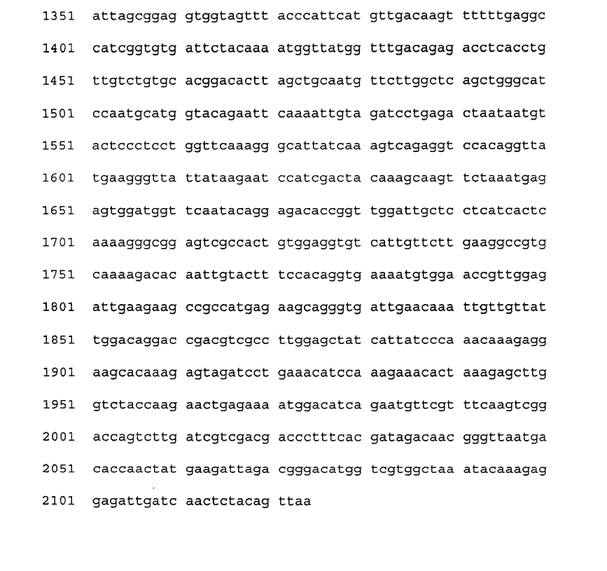

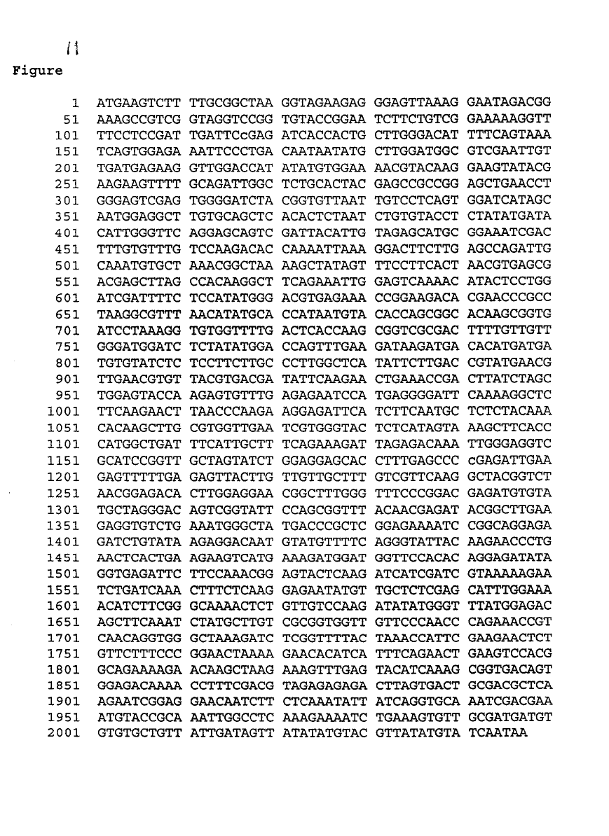

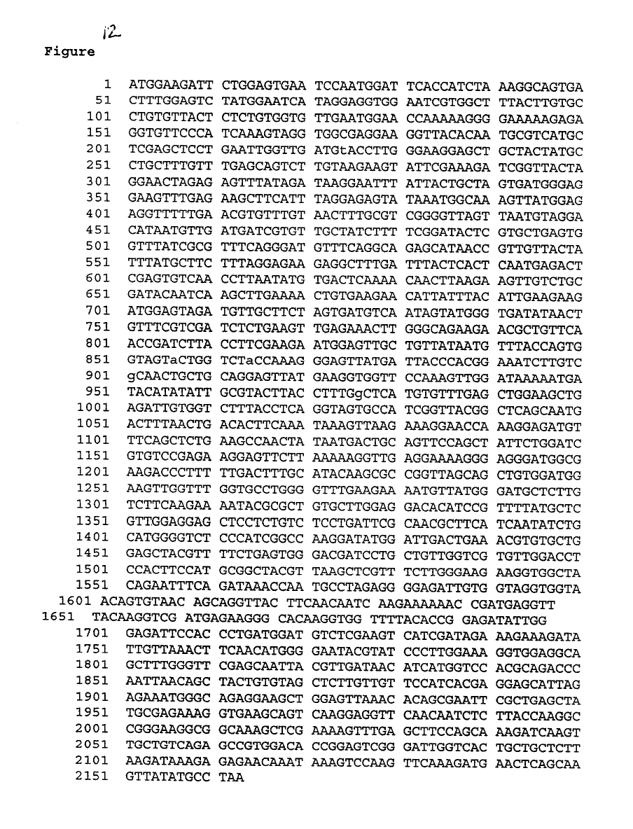

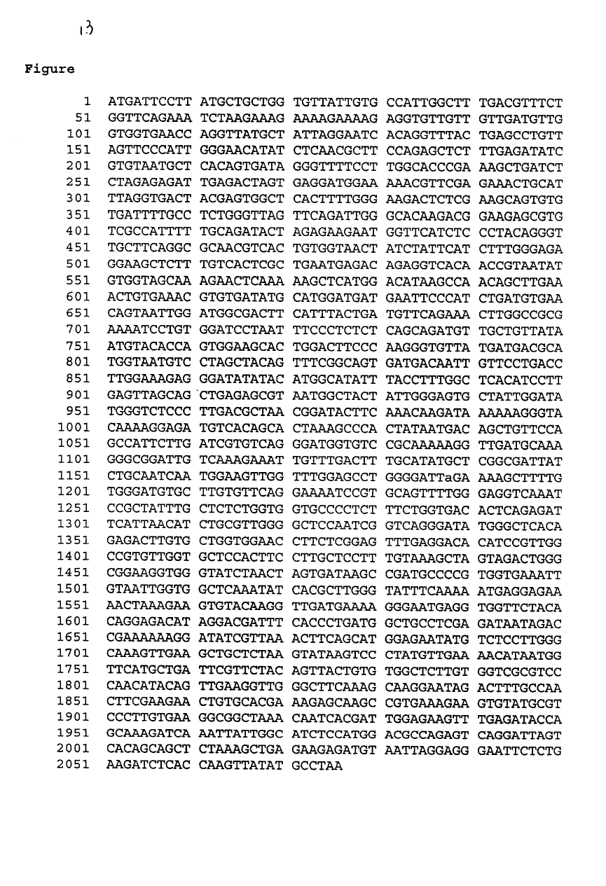

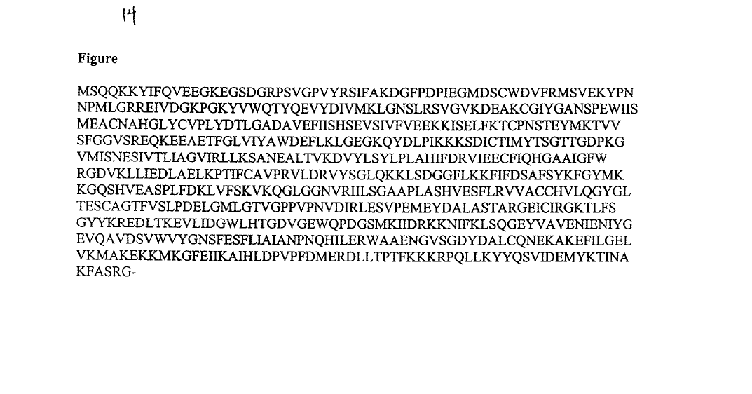

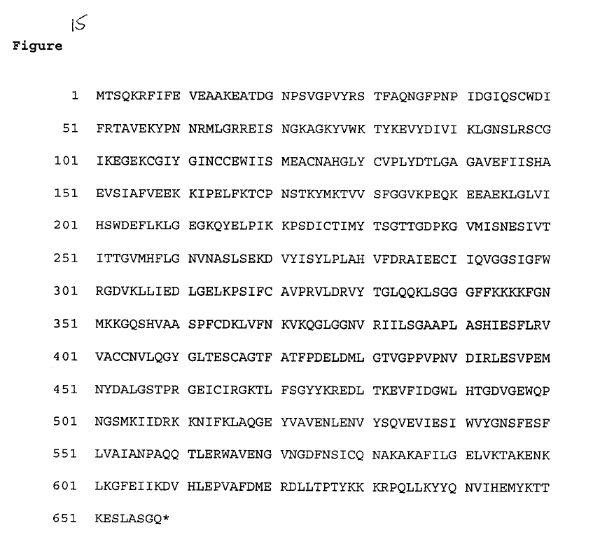

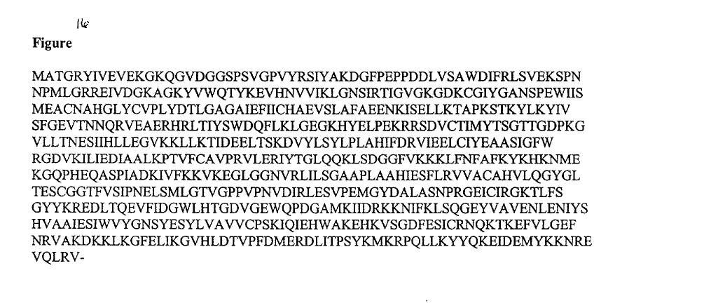

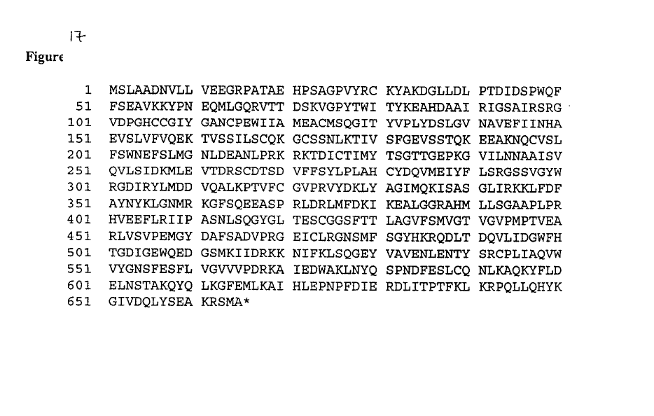

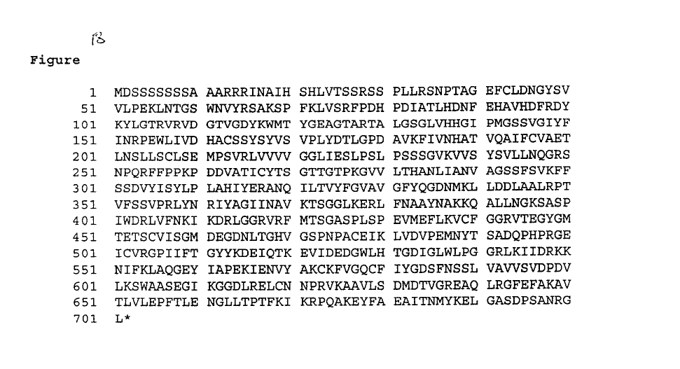

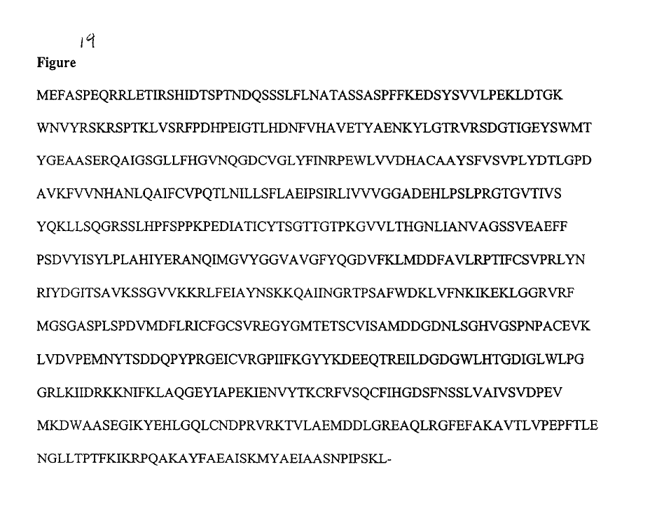

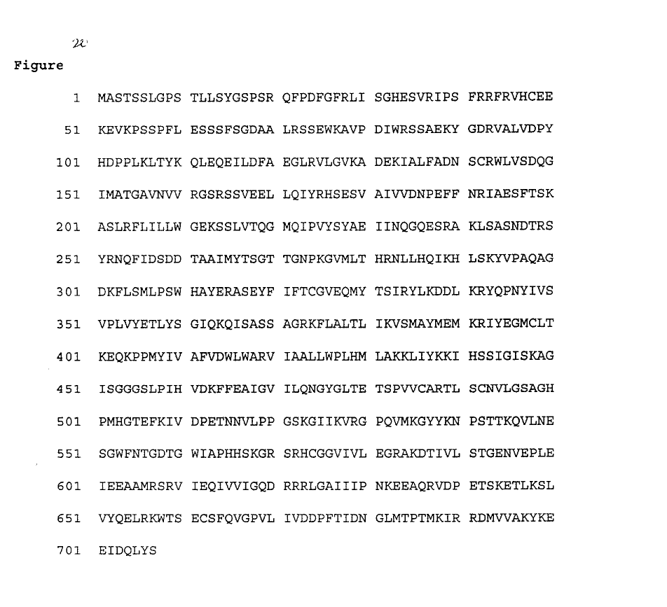

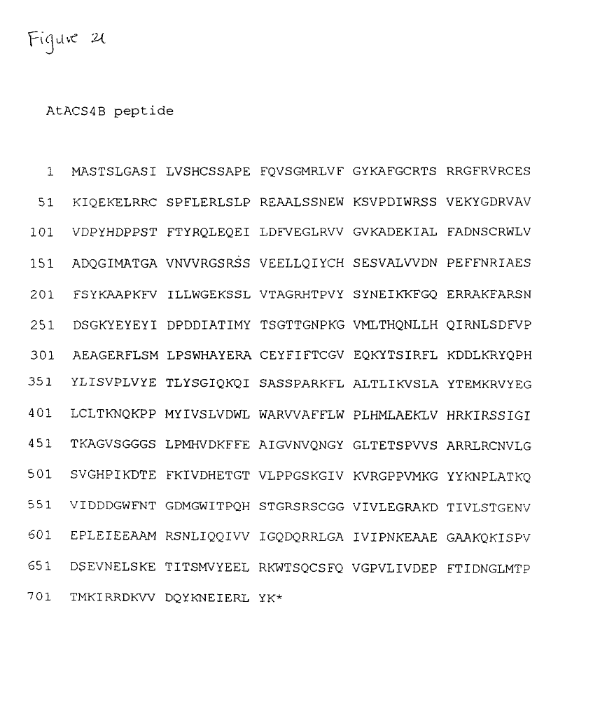

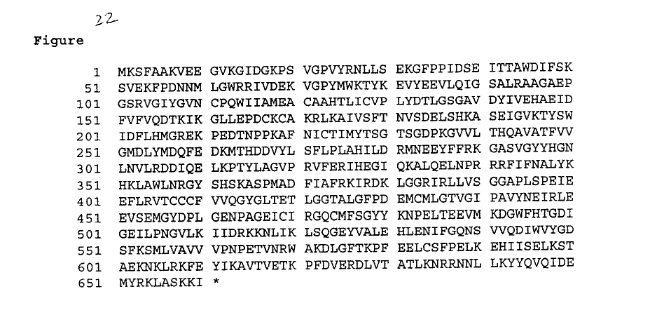

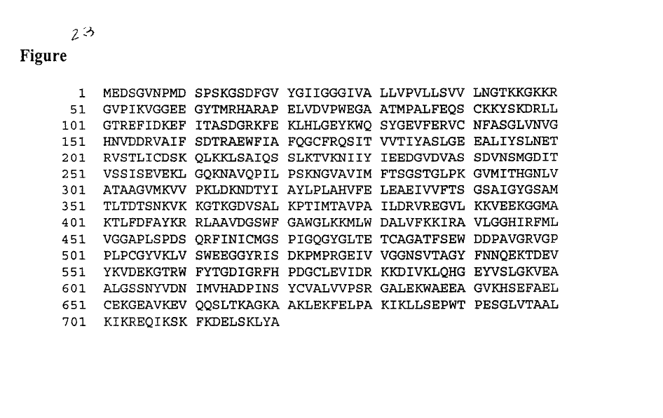

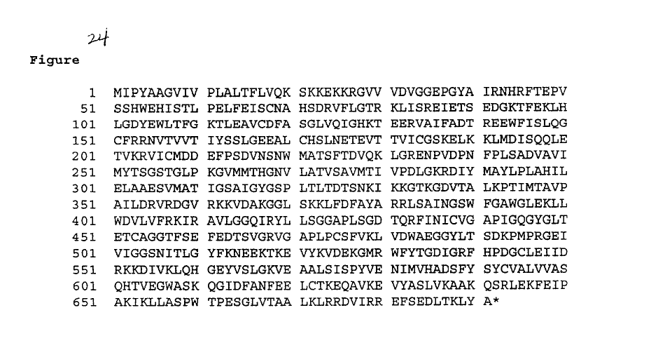

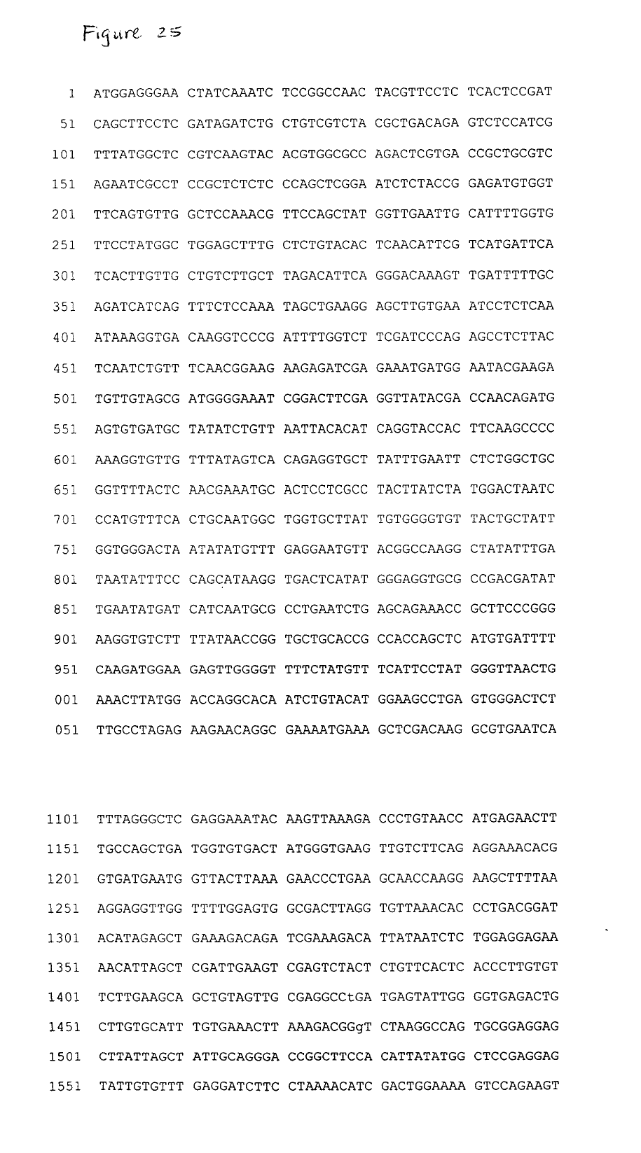

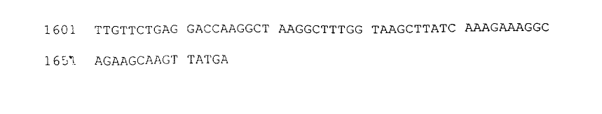

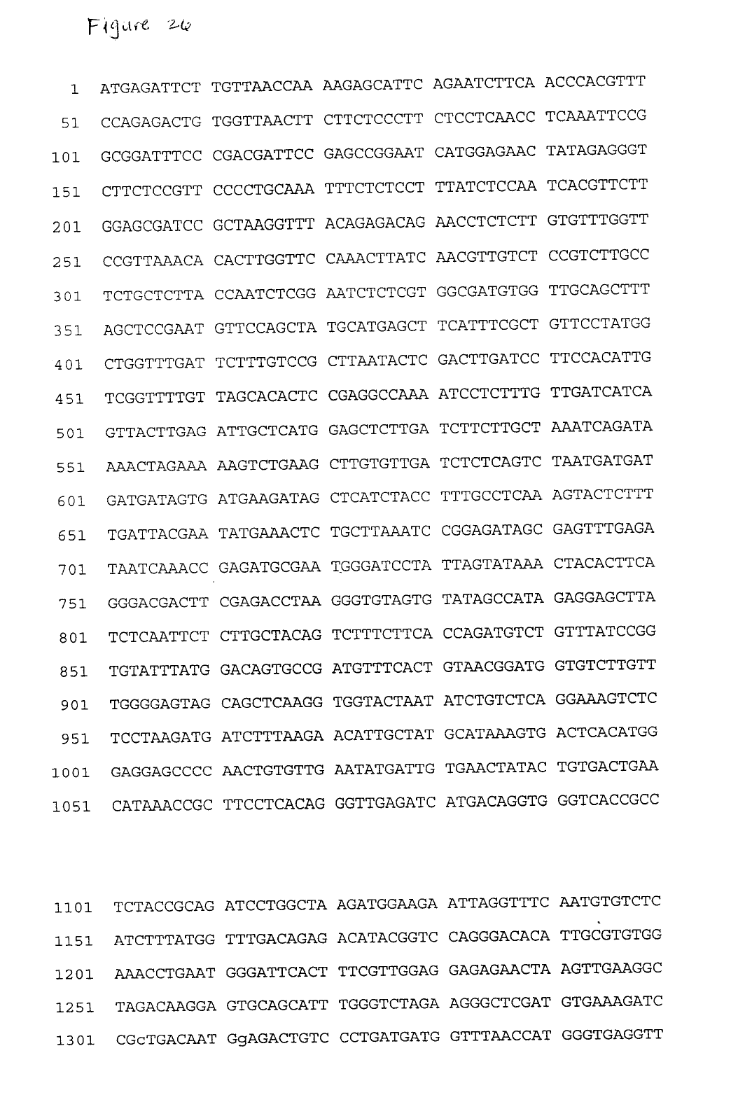

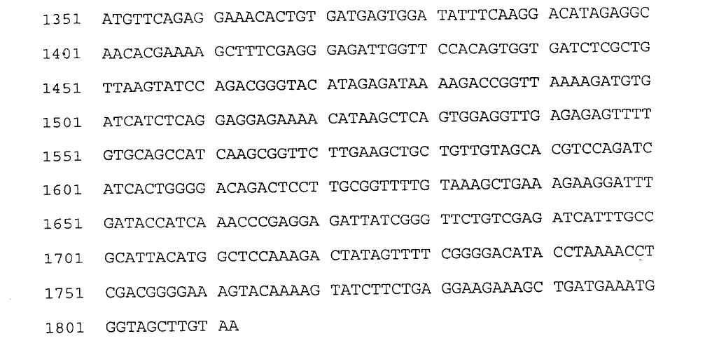

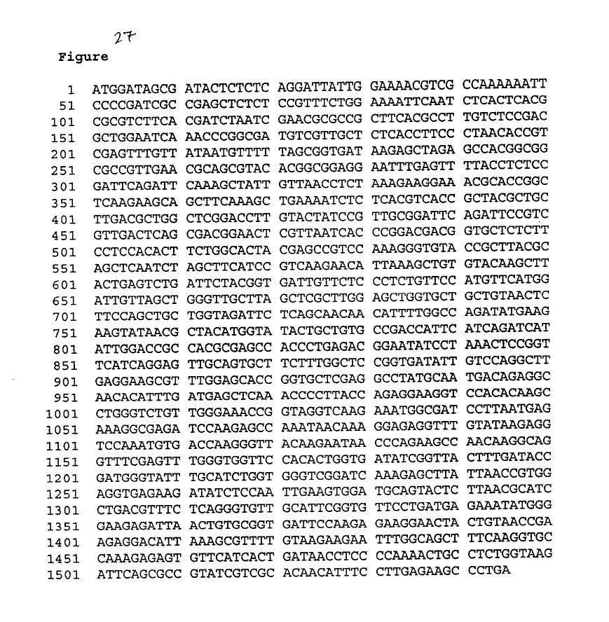

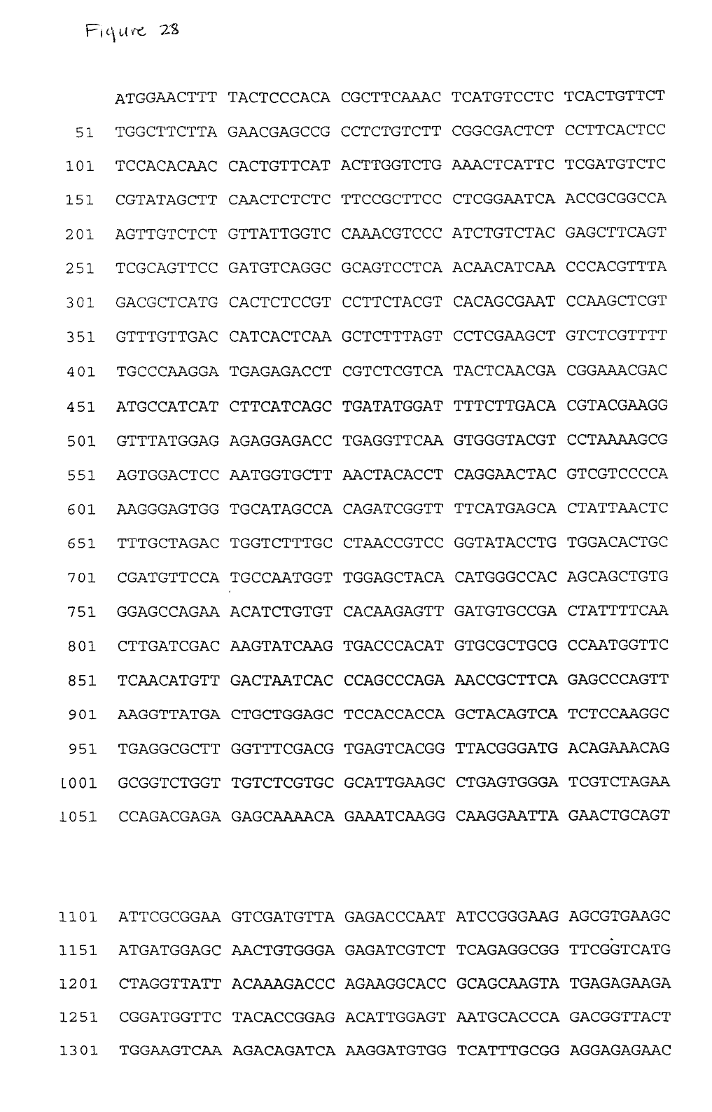

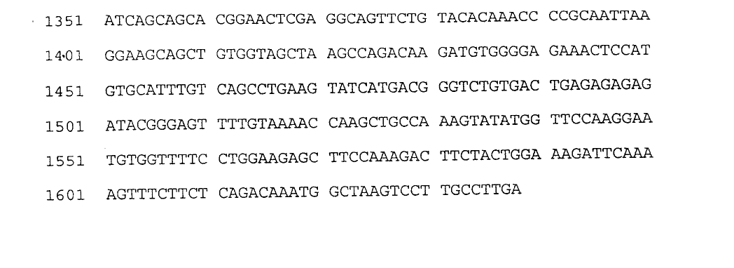

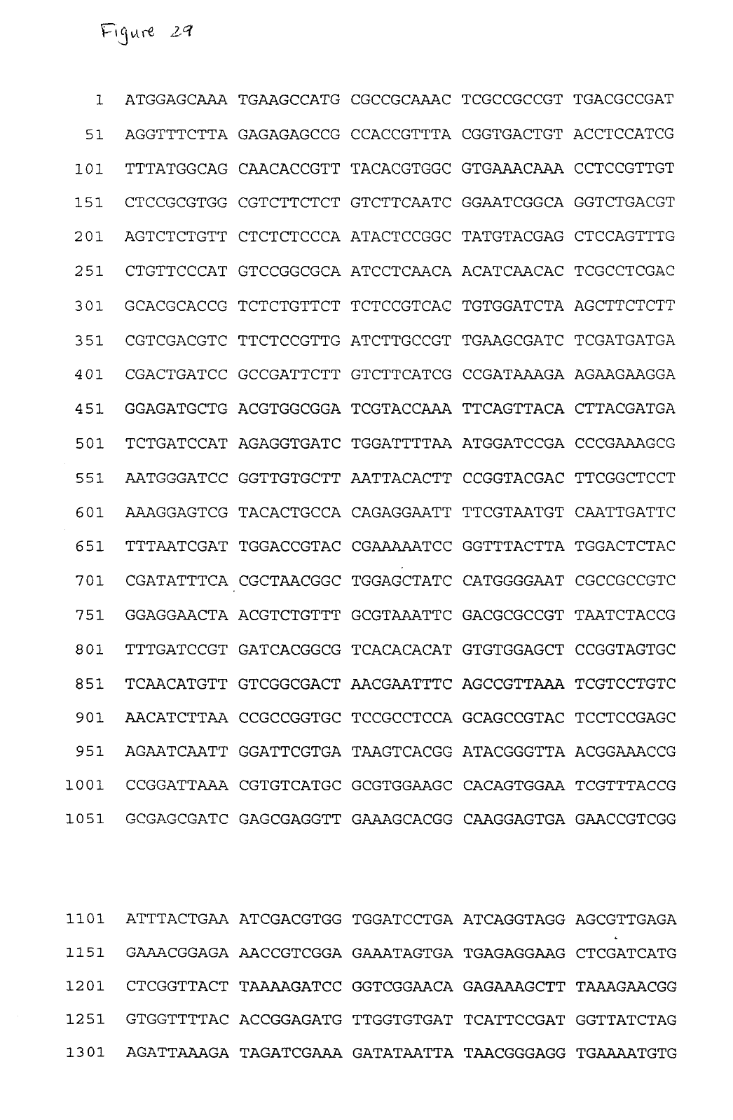

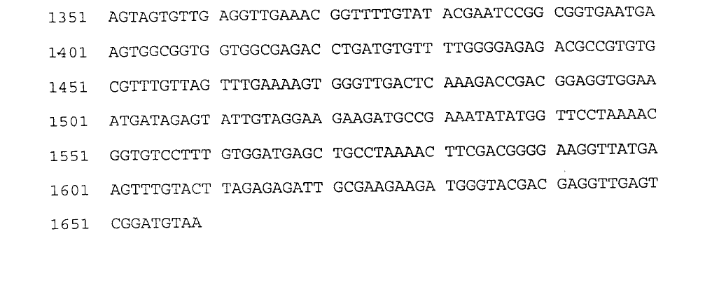

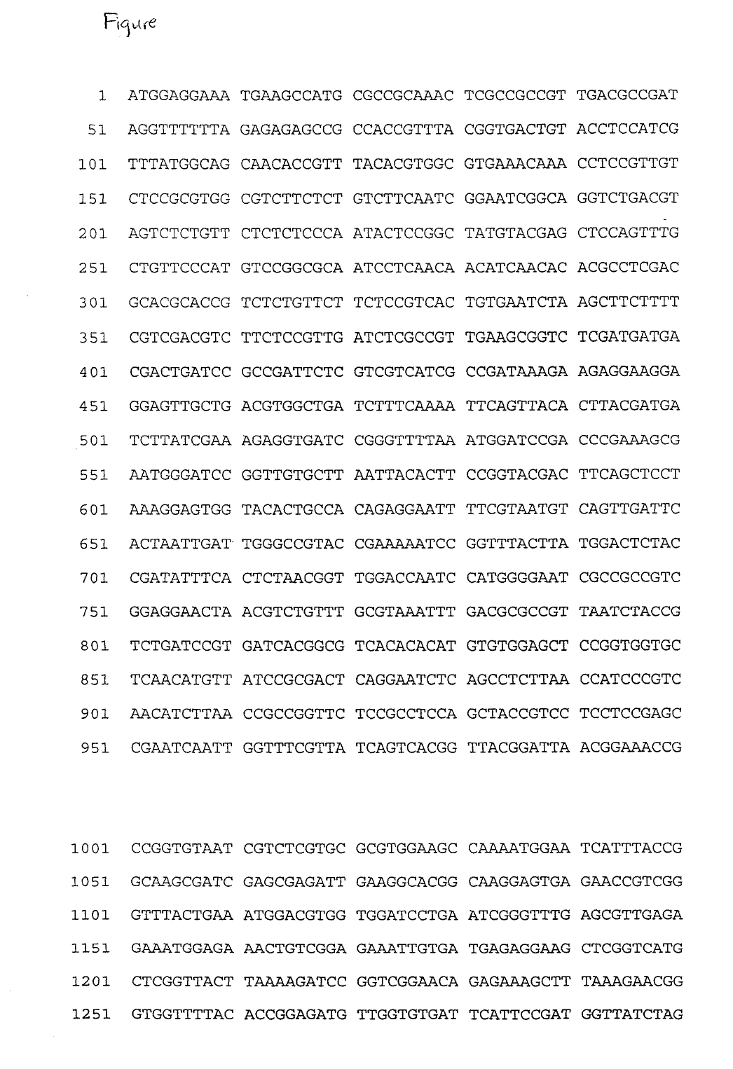

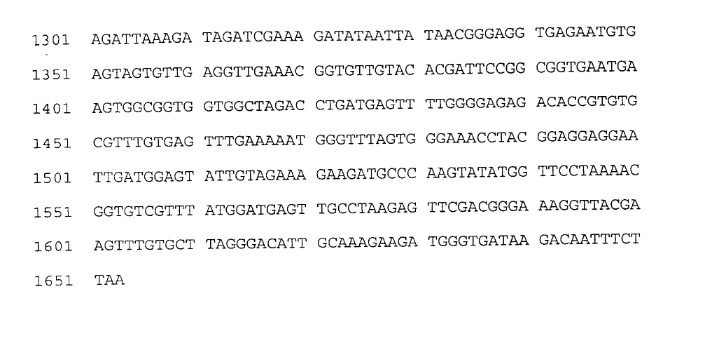

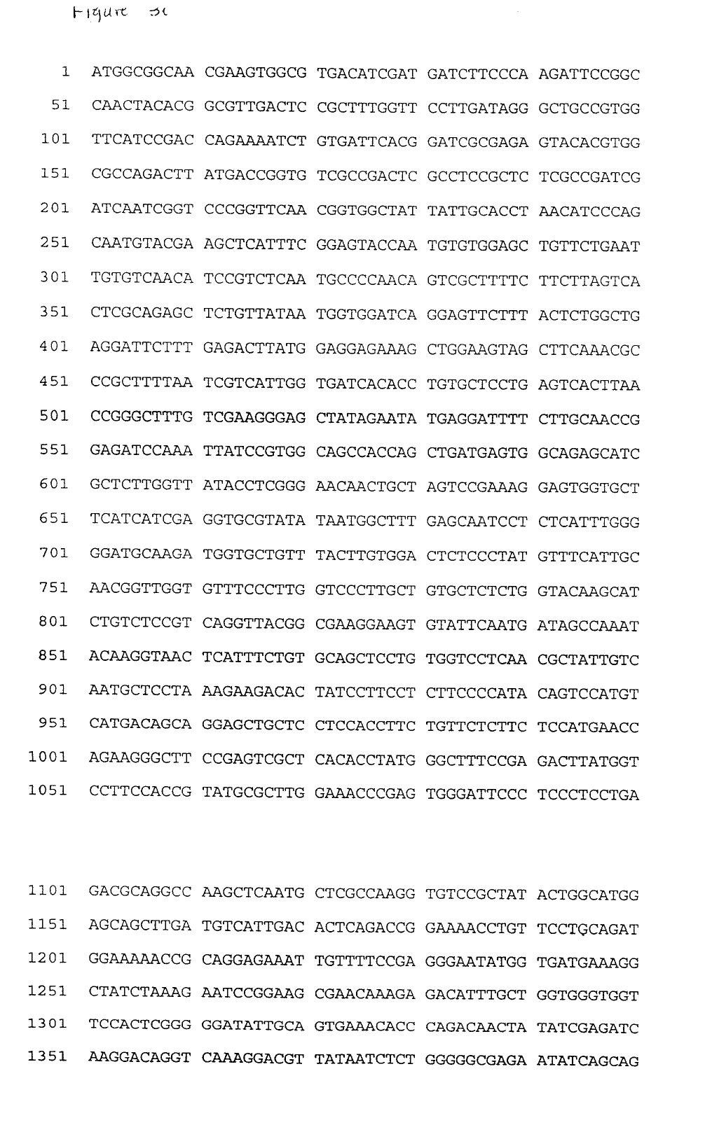

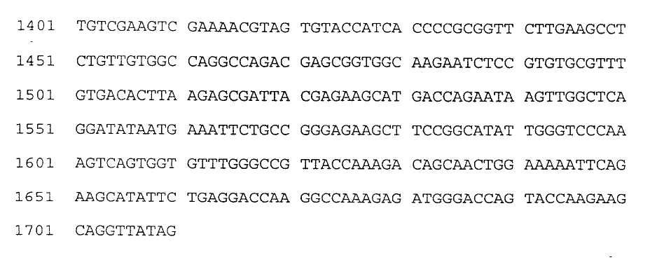

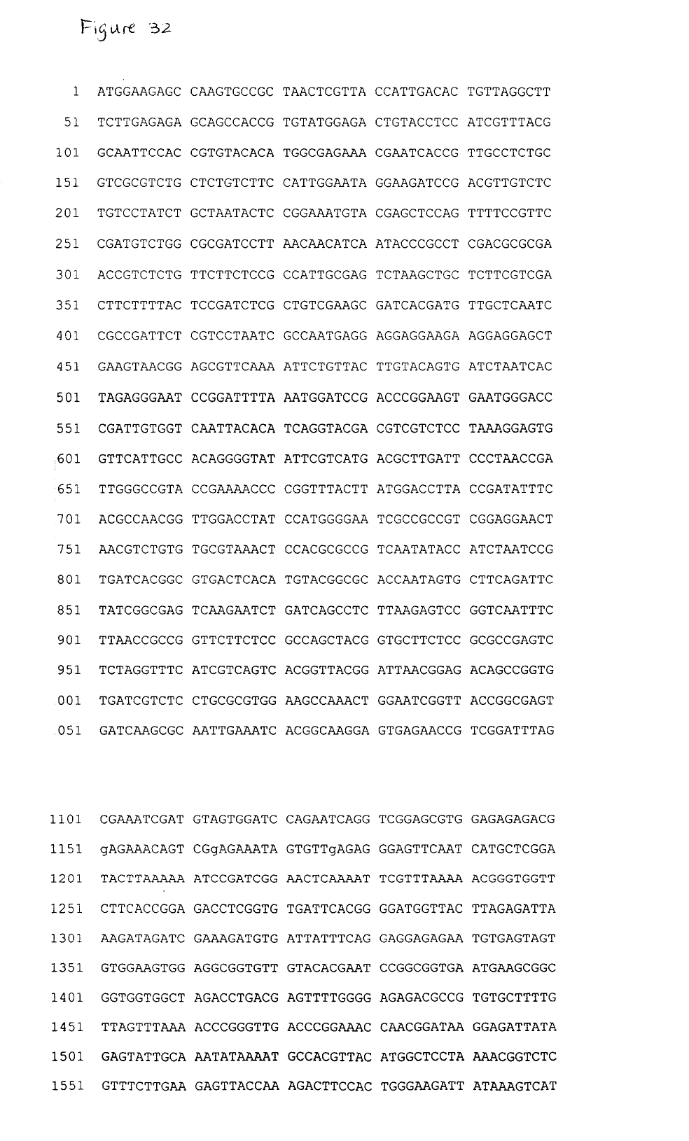

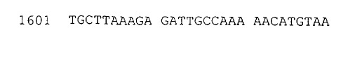

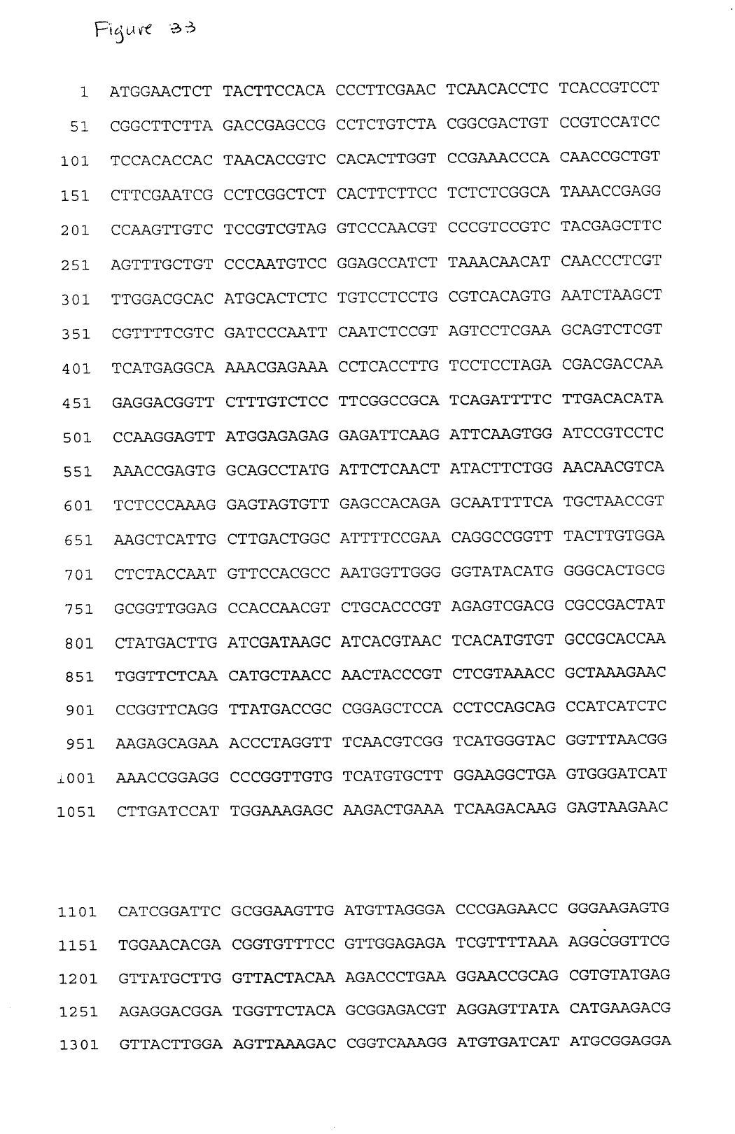

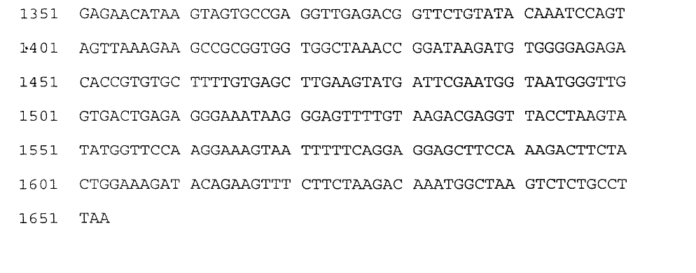

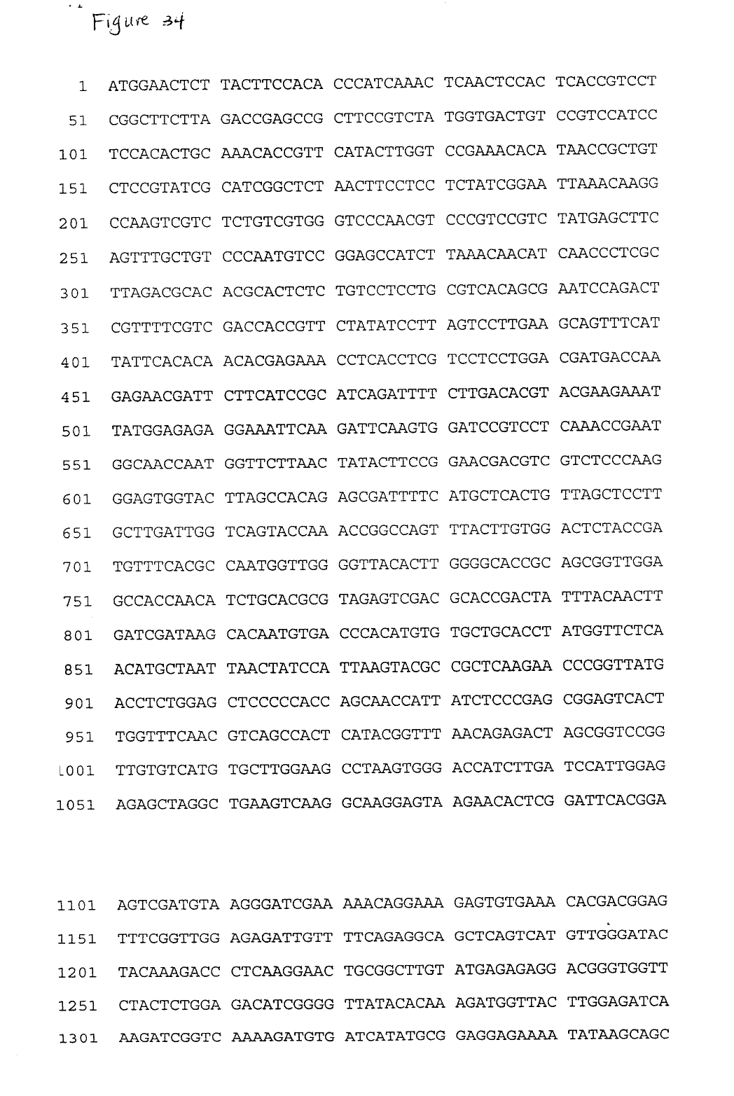

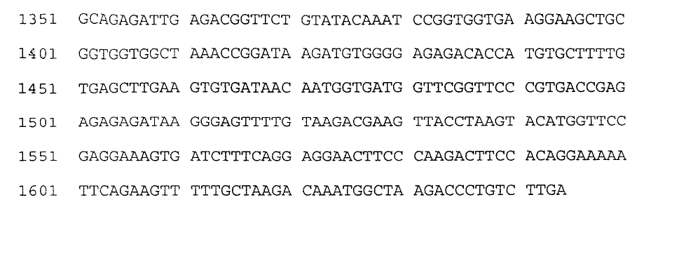

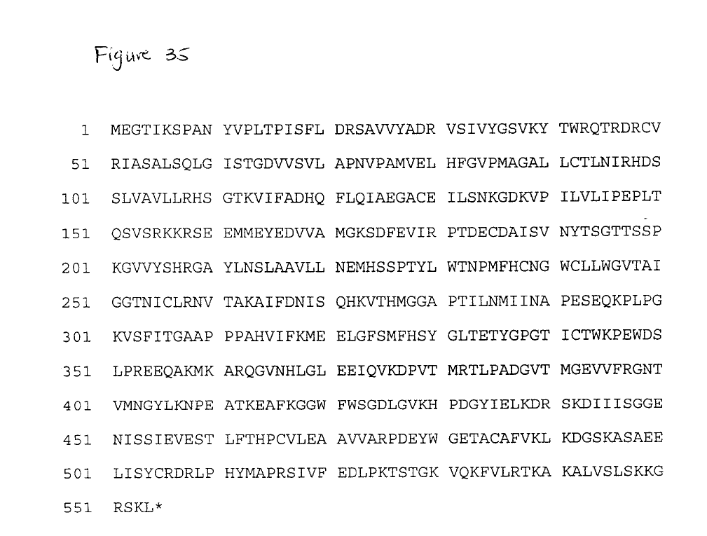

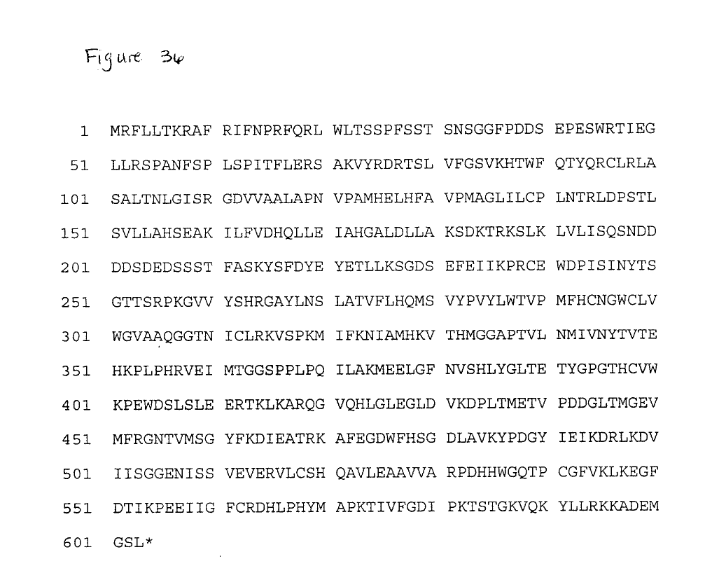

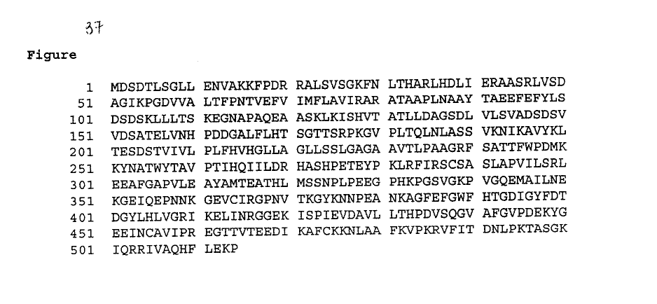

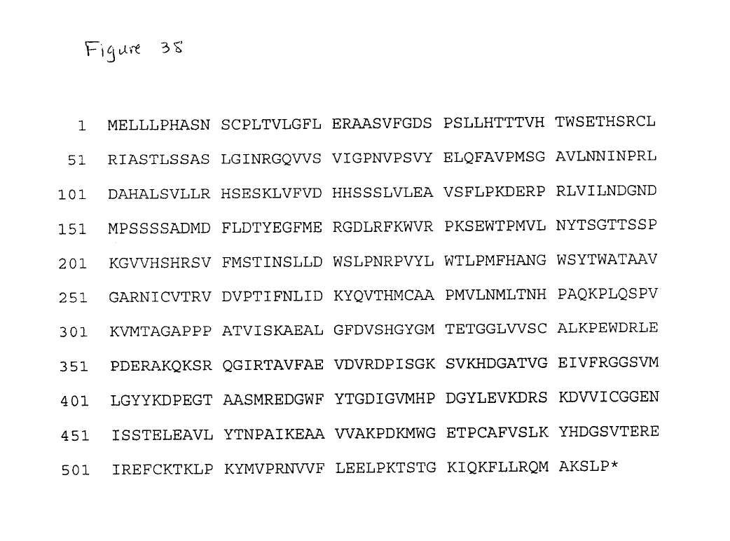

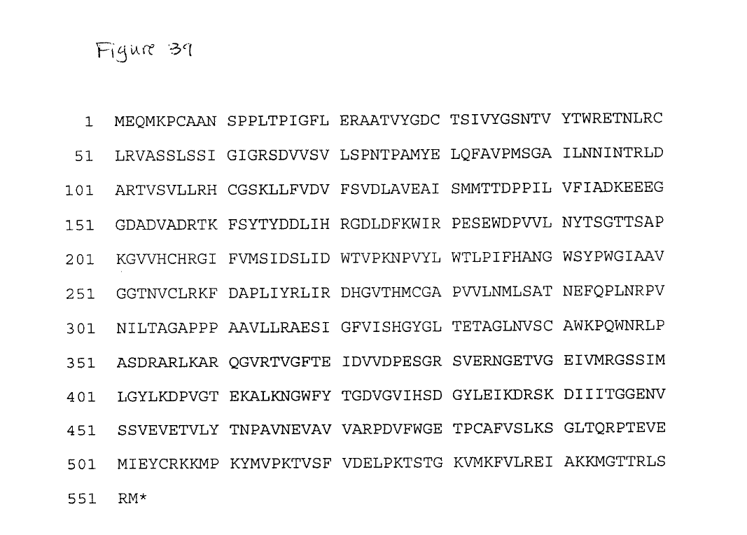

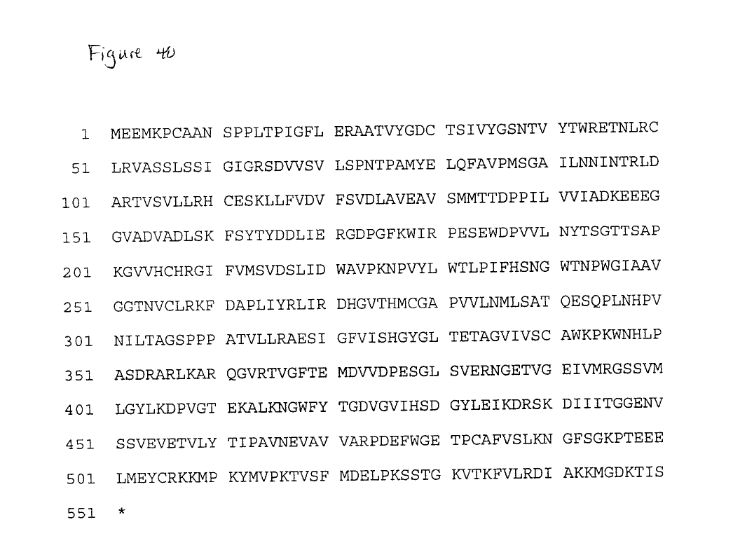

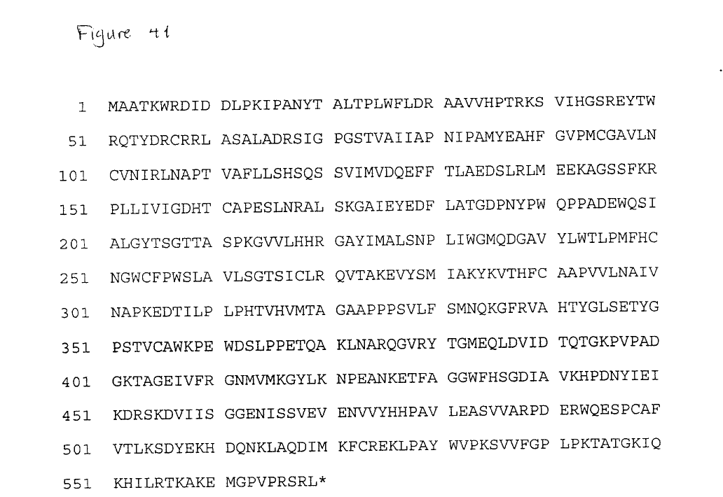

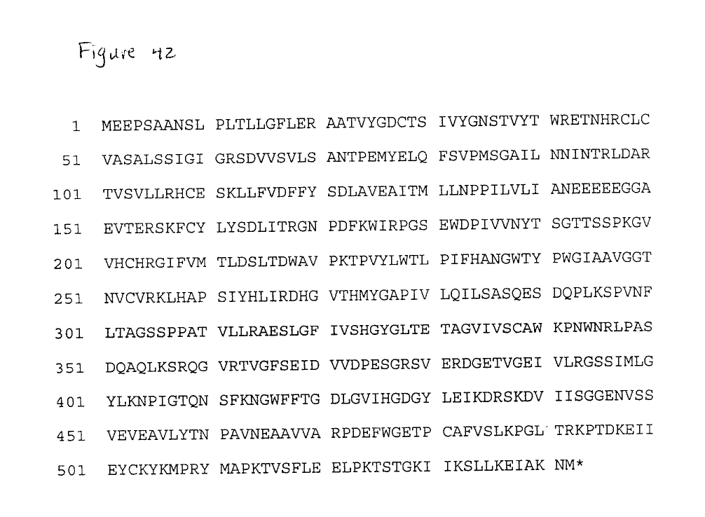

Accordingly, the present invention provides nucleic acids encoding plant ACSs (e.g., such as the nucleic acid sequences SEQ ID NOs: 1-11, as shown in FIGS. 3-13, or which encode amino acid sequences SEQ ID NOS: 12-22, as shown in FIGS. 14-24). Other embodiments of the present invention provide nucleic acid sequences that are capable of hybridizing to SEQ ID NOs: 1-11 under conditions of high to low stringency. In some embodiments, the hybridizing nucleic acid sequence encodes a protein that retains at least one biological activity of the naturally occurring ACS it is derived from. In preferred embodiments, hybridization conditions are based on the melting temperature (Tₘ) of the nucleic acid binding complex and confer a defined “stringency” as explained above.

In other embodiments of the present invention, variants of the disclosed ACSs are provided. In preferred embodiments, variants result from mutation, (i.e., a change in the nucleic acid sequence) and generally produce altered mRNAs or polypeptides whose structure or function may or may not be altered. Any given gene may have none, one, or many variant forms. Common mutational changes that give rise to variants are generally ascribed to deletions, additions or substitutions of nucleic acids. Each of these types of changes may occur alone, or in combination with the others, and at the rate of one or more times in a given sequence.

It is contemplated that is possible to modify the structure of a peptide having an activity (e.g., ACS activity) for such purposes as increasing synthetic activity or altering the affinity of the ACS for a particular fatty acid substrate. Such modified peptides are considered functional equivalents of peptides having an activity of an ACS as defined herein. A modified peptide can be produced in which the nucleotide sequence encoding the polypeptide has been altered, such as by substitution, deletion, or addition. In some preferred embodiments of the present invention, the alteration increases synthetic activity or alters the affinity of the ACS for a particular fatty acid substrate. In particularly preferred embodiments, these modifications do not significantly reduce the synthetic activity of the modified enzyme. In other words, construct “X” can be evaluated in order to determine whether it is a member of the genus of modified or variant ACSs of the present invention as defined functionally, rather than structurally. In preferred embodiments, the activity of variant ACSs is evaluated by the methods described in Examples 4 and 5. Accordingly, in some embodiments the present invention provides nucleic acids encoding plant acyl-CoA synthetases that complement yeast strain YB525. In other embodiments, the present invention provides nucleic acids encoding plant acyl-CoA synthetases that compete for the binding of fatty acid substrates with the proteins encoded by SEQ ID NOs: 1-11.

Moreover, as described above, variant forms of ACSs are also contemplated as being equivalent to those peptides and DNA molecules that are set forth in more detail herein. For example, it is contemplated that isolated replacement of a leucine with an isoleucine or valine, an aspartate with a glutamate, a threonine with a serine, or a similar replacement of an amino acid with a structurally related amino acid (i.e., conservative mutations) will not have a major effect on the biological activity of the resulting molecule. Accordingly, some embodiments of the present invention provide variants of ACSs disclosed herein containing conservative replacements. Conservative replacements are those that take place within a family of amino acids that are related in their side chains. Genetically encoded amino acids can be divided into four families: (1) acidic (aspartate, glutamate); (2) basic (lysine, arginine, histidine); (3) nonpolar (alanine, valine, leucine, isoleucine, proline, phenylalanine, methionine, tryptophan); and (4) uncharged polar (glycine, asparagine, glutamine, cysteine, serine, threonine, tyrosine). Phenylalanine, tryptophan, and tyrosine are sometimes classified jointly as aromatic amino acids. In similar fashion, the amino acid repertoire can be grouped as (1) acidic (aspartate, glutamate); (2) basic (lysine, arginine, histidine), (3) aliphatic (glycine, alanine, valine, leucine, isoleucine, serine, threonine), with serine and threonine optionally be grouped separately as aliphatic-hydroxyl; (4) aromatic (phenylalanine, tyrosine, tryptophan); (5) amide (asparagine, glutamine); and (6) sulfur-containing (cysteine and methionine) (e.g., Stryer ed., Biochemistry, pg. 17-21, 2nd ed, W H Freeman and Co., 1981). Whether a change in the amino acid sequence of a peptide results in a functional homolog can be readily determined by assessing the ability of the variant peptide to function in a fashion similar to the wild-type protein. Peptides having more than one replacement can readily be tested in the same manner.

More rarely, a variant includes “nonconservative” changes (e.g., replacement of a glycine with a tryptophan). Analogous minor variations can also include amino acid deletions or insertions, or both. Guidance in determining which amino acid residues can be substituted, inserted, or deleted without abolishing biological activity can be found using computer programs (e.g., LASERGENE software, DNASTAR Inc., Madison, Wis.).

As described in more detail below, variants may be produced by methods such as directed evolution or other techniques for producing combinatorial libraries of variants, described in more detail below. In still other embodiments of the present invention, the nucleotide sequences of the present invention may be engineered in order to alter an ACS coding sequence including, but not limited to, alterations that modify the cloning, processing, localization, secretion, and/or expression of the gene product. For example, mutations may be introduced using techniques that are well known in the art (e.g., site-directed mutagenesis to insert new restriction sites, alter glycosylation patterns, or change codon preference, etc.).

B. ACS Polypeptides

The family of ACS genes provided by the present invention represents a very diverse group of genes, as indicated by the results of the ACS amino acid sequence analysis summarized in FIG. 1 and Table 1. While half of the gene family members are nearly identical in length (approximately 665 amino acids) (AtACS1A, 1B, 1C, 2, and 5), the other half all contain N-terminal extensions of between 30 and 60 amino acid residues (AtACS3A, 3B, 4A, 4B, 6A, and 6B). As a group, the family of genes share only 30% identical amino acids and is clearly delineated into several distinct subgroupings. The number of ESTs associated with each of the ACS genes also varied considerably, with some genes represented by numerous ESTs and others not represented at all. Collectively, these observations support the biochemical evidence laid out in previous reports that the ACS gene family is responsible for providing acyl-CoA substrates for a number of distinct metabolic pathways that are carried out under conditions that vary considerably with respect to tissue type, cell type, and organelle, with varied levels of demand upon particular isoforms compared to others. It is interesting to note that all of the ACS amino acid sequences appear to lack a typical plastidial targeting consensus sequence, yet subsequent analysis has demonstrated that at least some of these ACSs can be imported into the chloroplast, and at least one ACS may be associated with the chloroplast envelope membranes (see Example 7).

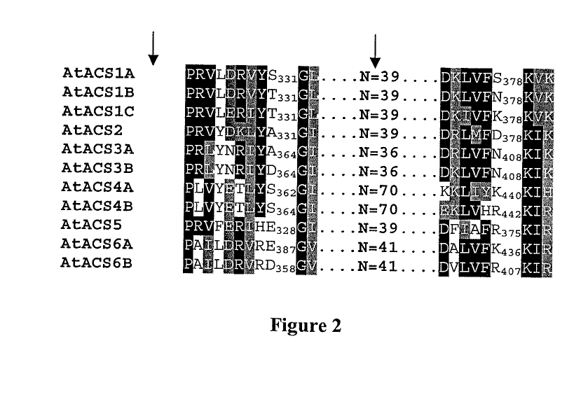

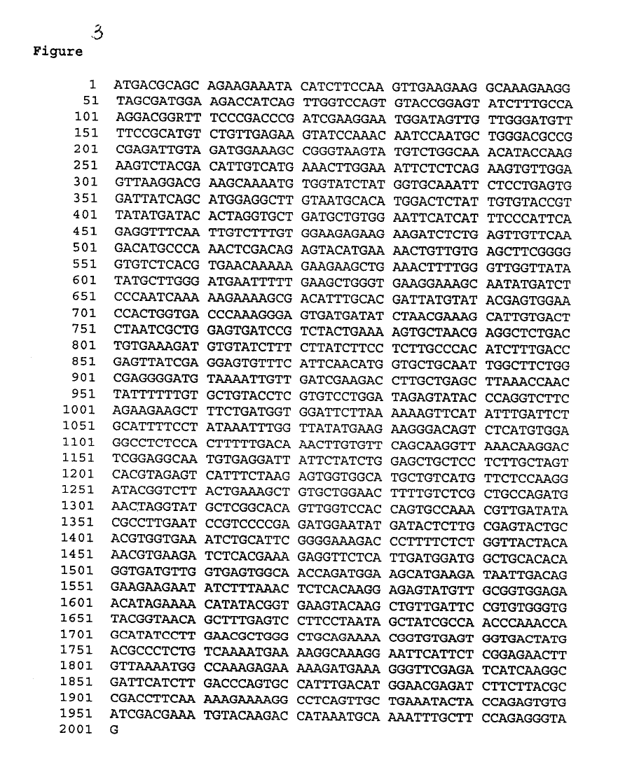

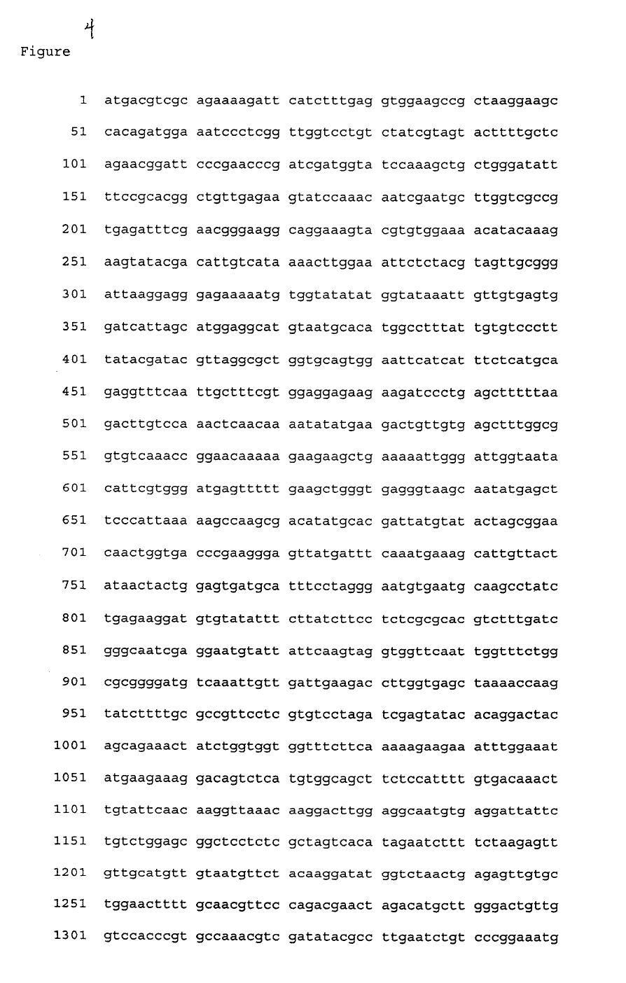

The degree of conservation of the deduced amino acid sequences of and around the insertional elements of each ACS gene of the present invention were also compared. The results of this comparison are shown in FIG. 2. The residues corresponding to the predicted borders of the insertional element are numbered and denoted with arrows. These residues were determined by comparing the sequences of the candidate ACS genes to those of the other AMP-BP genes that were identified in the original data base screen and which lacked the insertional element. For clarity, FIG. 2 displays only the first few amino acid residues that flank the upstream and downstream borders of the insertional region. Taking into account the N-terminal extensions present in some of the ACS genes, the comparison of the insertional element sequences confirmed the conservation of location of this element within the open reading frames of all members of this set of genes. The homology between the entire set of full-length insertional elements is quite weak, displaying approximately 30% identical amino acids between all eleven genes, which closely matches the degree of conservation between the eleven full-length proteins. Surprisingly, the regions immediately flanking the insertional element, are highly conserved across the whole family of eleven candidate ACS genes (see FIG. 2). These data suggest that amino acid residues encoded by the insertional element are necessary for proper ACS function in the plant, with the residues in the middle of the element evolving with the rest of the gene to diversify and specialize the enzymatic function of each gene, while the residues near the borders of the element constitute a more invariable region of the enzyme that is essential to the core reaction.

Accordingly, the present invention also provides ACS polypeptides (e.g., SEQ ID NOs: 12-22 as shown in FIGS. 14-24). Still further embodiments of the present invention provide fragments, fusion proteins or functional equivalents of ACSs. Functional equivalents of ACSs may be screened for as described in Examples 4 and 5. In still other embodiments of the present invention, nucleic acid sequences corresponding to a selected ACS may be used to generate recombinant DNA molecules that direct the expression of an ACS and variants in appropriate host cells. In some embodiments of the present invention, the polypeptide may be a naturally purified product, while in other embodiments it may be a product of chemical synthetic procedures, and in still other embodiments it may be produced by recombinant techniques using a prokaryotic or eukaryotic host cell (e.g., by bacterial cells in culture). In other embodiments, the polypeptides of the invention may also include an initial methionine amino acid residue.

In one embodiment of the present invention, due to the inherent degeneracy of the genetic code, DNA sequences other than SEQ ID NOs: 1-11 encoding substantially the same or a functionally equivalent amino acid sequence, may be used to clone and express an ACS. In general, such nucleic acid sequences hybridize to SEQ ID NOs: 1-11 under conditions of high to low stringency as described above. As will be understood by those of skill in the art, it may be advantageous to produce ACS-encoding nucleotide sequences possessing non-naturally occurring codons. Therefore, in some preferred embodiments, codons preferred by a particular prokaryotic or eukaryotic host are selected, for example, to increase the rate of ACS expression or to produce recombinant RNA transcripts having desirable properties, such as increased synthetic activity or altered affinity of the ACS for a particular fatty acid substrate.

II. Uses of ACS Polynucleotides and Polypeptides

1. Vectors for Expression of ACSs

In some embodiments of the present invention, the ACS nucleic acids are used to construct vectors for the expression of ACS polypeptides. Accordingly, the nucleic acids of the present invention may be employed for producing polypeptides by recombinant techniques. Thus, for example, the nucleic acid may be included in any one of a variety of expression vectors for expressing a polypeptide.

In some embodiments of the present invention, vectors are provided for the transfection of plant hosts to create transgenic plants. In general, these vectors comprise an ACS nucleic acid (e.g., SEQ ID NOs: 1-11) operably linked to a promoter and other regulatory sequences (e.g., enhancers, polyadenylation signals, etc.) required for expression in a plant. The ACS nucleic acid can be oriented to produce sense or antisense transcripts, depending on the desired use. In some embodiments, the promoter is a constitutive promoter (e.g., superpromoter or SD promoter). In other embodiments, the promoter is a seed specific promoter (e.g., phaseolin promoter [See e.g., U.S. Pat. No. 5,589,616, incorporated herein by reference], napin promoter [See e.g., U.S. Pat. No. 5,608,152, incorporated herein by reference], or acyl-CoA carrier protein promoter [See e.g., 5,767,363, incorporated herein by reference]).

In some preferred embodiments, the vector is adapted for use in an Agrobacterium mediated transfection process (See e.g., U.S. Pat. Nos. 5,981,839; 6,051,757; 5,981,840; 5,824,877; and 4,940,838; all of which are incorporated herein by reference). Construction of recombinant Ti and Ri plasmids in general follows methods typically used with the more common bacterial vectors, such as pBR322. Additional use can be made of accessory genetic elements sometimes found with the native plasmids and sometimes constructed from foreign sequences. These may include but are not limited to structural genes for antibiotic resistance as selection genes.

There are two systems of recombinant Ti and Ri plasmid vector systems now in use. The first system is called the “cointegrate” system. In this system, the shuttle vector containing the gene of interest is inserted by genetic recombination into a non-oncogenic Ti plasmid that contains both the cis-acting and trans-acting elements required for plant transformation as, for example, in the pMLJ1 shuttle vector and the non-oncogenic Ti plasmid pGV3850. The second system is called the “binary” system in which two plasmids are used; the gene of interest is inserted into a shuttle vector containing the cis-acting elements required for plant transformation. The other necessary functions are provided in trans by the non-oncogenic Ti plasmid as exemplified by the pBIN19 shuttle vector and the non-oncogenic Ti plasmid PAL4404. Some of these vectors are commercially available.

It may be desirable to target the nucleic acid sequence of interest to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences, as previously described (U.S. Pat. No. 5,501,967, the entire contents of which are herein incorporated by reference). One of skill in the art knows that homologous recombination may be achieved using targeting vectors which contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

The nucleic acids of the present invention may also be utilized to construct vectors derived from plant (+) RNA viruses (e.g., brome mosaic virus, tobacco mosaic virus, alfalfa mosaic virus, cucumber mosaic virus, tomato mosaic virus, and combinations and hybrids thereof). Generally, the inserted ACS polynucleotide can be expressed from these vectors as a fusion protein (e.g., coat protein fusion protein) or from its own subgenomic promoter or other promoter. Methods for the construction and use of such viruses are described in U.S. Pat. Nos. 5,846,795; 5,500,360; 5,173,410; 5,965,794; 5,977,438; and 5,866,785, all of which are incorporated herein by reference.

Alternatively, vectors can be constructed for expression in hosts other plants (e.g., prokaryotic cells such as E. coli, yeast cells, C. elegans, and mammalian cell culture cells). In some embodiments of the present invention, vectors include, but are not limited to, chromosomal, nonchromosomal and synthetic DNA sequences (e.g., derivatives of SV40, bacterial plasmids, phage DNA; baculovirus, yeast plasmids, vectors derived from combinations of plasmids and phage DNA, and viral DNA such as vaccinia, adenovirus, fowl pox virus, and pseudorabies). Large numbers of suitable vectors that are replicable and viable in the host are known to those of skill in the art, and are commercially available. Any other plasmid or vector may be used as long as they are replicable and viable in the host.

In some preferred embodiments of the present invention, bacterial expression vectors comprise an origin of replication, a suitable promoter and optionally an enhancer, and also any necessary ribosome binding sites, polyadenylation sites, transcriptional termination sequences, and 5′ flanking nontranscribed sequences. Promoters useful in the present invention include, but are not limited to, retroviral LTRs, SV40 promoter, CMV promoter, RSV promoter, E. coli lac or trp promoters, phage lambda P_{L} and P_{R} promoters, T3, SP6 and T7 promoters. In other embodiments of the present invention, recombinant expression vectors include origins of replication and selectable markers, (e.g., tetracycline or ampicillin resistance in E. coli, or neomycin phosphotransferase gene for selection in eukaryotic cells).

2. Expression of ACSs in Transgenic Plants

Vectors described above can be utilized to express the ACSs of the present invention in transgenic plants. A variety of methods are known for producing transgenic plants.

In some embodiments, Agrobacterium mediated transfection is utilized to create transgenic plants. Since most dicotyledonous plant are natural hosts for Agrobacterium, almost every dicotyledonous plant may be transformed by Agrobacterium in vitro. Although monocotyledonous plants, and in particular, cereals and grasses, are not natural hosts to Agrobacterium, work to transform them using Agrobacterium has also been carried out (Hooykas-Van Slogteren et al. (1984) Nature 311:763-764). Plant genera that may be transformed by Agrobacterium include Arabidopsis, Chrysanthemum, Dianthus, Gerbera, Euphorbia, Pelaronium, Ipomoea, Passiflora, Cyclamen, Malus, Prunus, Rosa, Rubus, Populus, Santalum, Allium, Lilium, Narcissus, Ananas, Arachis, Phaseolus and Pisum.

For transformation with Agrobacterium, disarmed Agrobacterium cells are transformed with recombinant Ti plasmids of Agrobacterium tumefaciens or Ri plasmids of Agrobacterium rhizogenes (such as those described in U.S. Pat. No. 4,940,838, the entire contents of which are herein incorporated by reference). The nucleic acid sequence of interest is then stably integrated into the plant genome by infection with the transformed Agrobacterium strain. For example, heterologous nucleic acid sequences have been introduced into plant tissues using the natural DNA transfer system of Agrobacterium tumefaciens and Agrobacterium rhizogenes bacteria (for review, see Klee et al. (1987) Ann. Rev. Plant Phys. 38:467-486).

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (a) that the plant cells or tissues can be transformed by Agrobacterium and (b) that the transformed cells or tissues can be induced to regenerate into whole plants. The third method is transformation of seeds, apices or meristems with Agrobacterium. This method requires micropropagation.

One of skill in the art knows that the efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., (1987) Plant Molec. Biol. 8:291-298). Alternatively, transformation efficiency may be enhanced by wounding the target tissue to be transformed. Wounding of plant tissue may be achieved, for example, by punching, maceration, bombardment with microprojectiles, etc. (See e.g., Bidney et al., (1992) Plant Molec. Biol. 18:301-313).

In still further embodiments, the plant cells are transfected with vectors via particle bombardment (i.e., with a gene gun). Particle mediated gene transfer methods are known in the art, are commercially available, and include, but are not limited to, the gas driven gene delivery instrument descried in McCabe, U.S. Pat. No. 5,584,807, the entire contents of which are herein incorporated by reference. This method involves coating the nucleic acid sequence of interest onto heavy metal particles, and accelerating the coated particles under the pressure of compressed gas for delivery to the target tissue.

Other particle bombardment methods are also available for the introduction of heterologous nucleic acid sequences into plant cells. Generally, these methods involve depositing the nucleic acid sequence of interest upon the surface of small, dense particles of a material such as gold, platinum, or tungsten. The coated particles are themselves then coated onto either a rigid surface, such as a metal plate, or onto a carrier sheet made of a fragile material such as mylar. The coated sheet is then accelerated toward the target biological tissue. The use of the flat sheet generates a uniform spread of accelerated particles which maximizes the number of cells receiving particles under uniform conditions, resulting in the introduction of the nucleic acid sample into the target tissue.

Plants, plant cells and tissues transformed with a heterologous nucleic acid sequence of interest are readily detected using methods known in the art including, but not limited to, restriction mapping of the genomic DNA, PCR-analysis, DNA-DNA hybridization, DNA-RNA hybridization, DNA sequence analysis and the like.

Additionally, selection of transformed plant cells may be accomplished using a selection marker gene. It is preferred, though not necessary, that a selection marker gene be used to select transformed plant cells. A selection marker gene may confer positive or negative selection.

A positive selection marker gene may be used in constructs for random integration and site-directed integration. Positive selection marker genes include antibiotic resistance genes, and herbicide resistance genes and the like. In one embodiment, the positive selection marker gene is the NPTII gene which confers resistance to geneticin (G418) or kanamycin. In another embodiment the positive selection marker gene is the HPT gene which confers resistance to hygromycin. The choice of the positive selection marker gene is not critical to the invention as long as it encodes a functional polypeptide product. Positive selection genes known in the art include, but are not limited to, the ALS gene (chlorsulphuron resistance), and the DHFR-gene (methothrexate resistance).

A negative selection marker gene may also be included in the constructs. The use of one or more negative selection marker genes in combination with a positive selection marker gene is preferred in constructs used for homologous recombination. Negative selection marker genes are generally placed outside the regions involved in the homologous recombination event. The negative selection marker gene serves to provide a disadvantage (preferably lethality) to cells that have integrated these genes into their genome in an expressible manner. Cells in which the targeting vectors for homologous recombination are randomly integrated in the genome will be harmed or killed due to the presence of the negative selection marker gene. Where a positive selection marker gene is included in the construct, only those cells having the positive selection marker gene integrated in their genome will survive.

The choice of the negative selection marker gene is not critical to the invention as long as it encodes a functional polypeptide in the transformed plant cell. The negative selection gene may for instance be chosen from the aux-2 gene from the Ti-plasmid of Agrobacterium, the tk-gene from SV40, cytochrome P450 from Streptomyces griseolus, the Adh-gene from Maize or Arabidopsis, etc. Any gene encoding an enzyme capable of converting a substance which is otherwise harmless to plant cells into a substance which is harmful to plant cells may be used.

It is contemplated that the ACS polynucleotides of the present invention may be utilized to either increase or decrease the level of ACS mRNA and/or protein in transfected cells as compared to the levels in wild-type cells. Accordingly, in some embodiments, expression in plants by the methods described above leads to the over-expression of ACS in transgenic plants, plant tissues, or plant cells. The present invention is not limited to any particular mechanism. Indeed, an understanding of a mechanism is not required to practice the present invention. However, it is contemplated that over-expression of the ACS polynucleotides of the present invention will overcome limitations in the accumulation of fatty acids in oilseeds.

In other embodiments of the present invention, the ACS polynucleotides are utilized to decrease the level of ACS protein or mRNA in transgenic plants, plant tissues, or plant cells as compared to wild-type plants, plant tissues, or plant cells. One method of reducing ACS expression utilizes expression of antisense transcripts. Antisense RNA has been used to inhibit plant target genes in a tissue-specific manner (e.g., van der Krol et al (1988) Biotechniques 6:958-976). Antisense inhibition has been shown using the entire cDNA sequence as well as a partial cDNA sequence (e.g., Sheehy et al. (1988) Proc. Natl. Acad. Sci. USA 85:8805-8809; Cannon et al. (1990) Plant Mol. Biol. 15:39-47). There is also evidence that 3′ non-coding sequence fragment and 5′ coding sequence fragments, containing as few as 41 base-pairs of a 1.87 kb cDNA, can play important roles in antisense inhibition (Ch'ng et al. (1989)Proc. Natl. Acad. Sci. USA 86:10006-10010).

Accordingly, in some embodiments, the ACS nucleic acids of the present invention (e.g., SEQ ID NOs: 1-11, and fragments and variants thereof) are oriented in a vector and expressed so as to produce antisense transcripts. To accomplish this, a nucleic acid segment from the desired gene is cloned and operably linked to a promoter such that the antisense strand of RNA will be transcribed. The expression cassette is then transformed into plants and the antisense strand of RNA is produced. The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other proteins within a family of genes exhibiting homology or substantial homology to the target gene.

Furthermore, for antisense suppression, the introduced sequence also need not be fill length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of at least about 500 nucleotides is especially preferred.

Catalytic RNA molecules or ribozymes can also be used to inhibit expression of the target gene or genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes have been identified. One class of ribozymes is derived from a number of small circular RNAs which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, Solanum nodiflorum mottle virus and subterranean clover mottle virus. The design and use of target RNA-specific ribozymes is described in Haseloff, et al., Nature 334:585-591 (1988).

Another method of reducing ACS expression utilizes the phenomenon of cosuppression or gene silencing (See e.g., U.S. Pat. No. 6,063,947, incorporated herein by reference). The phenomenon of cosuppression has also been used to inhibit plant target genes in a tissue-specific manner. Cosuppression of an endogenous gene using a full-length cDNA sequence as well as a partial cDNA sequence (730 bp of a 1770 bp cDNA) are known (e.g., Napoli et al. (1990) Plant Cell 2:279-289; van der Krol et al. (1990) Plant Cell 2:291-299; Smith et al., (1990) Mol. Gen. Genetics 224:477-481). Accordingly, in some embodiments the Arabidopsis ACS nucleic acids (e.g., SEQ ID NOs: 1-10, and fragments and variants thereof) are expressed in another species of plant to effect cosuppression of a homologous gene.

Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For cosuppression, the introduced sequence in the expression cassette, needing less than absolute identity, also need not be full length, relative to either the primary transcription product or fully processed mRNA. This may be preferred to avoid concurrent production of some plants which are over-expressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used.

3. Other Host Cells and Systems for Production of ACSs

The present invention also contemplates that the vectors described above can be utilized to express plant ACS genes and variants in prokaryotic and eukaryotic cells. In some embodiments of the present invention, the host cell can be a prokaryotic cell (e.g., a bacterial cell). Specific examples of host cells include, but are not limited to, E. coli, Salmonella typhimurium, Bacillus subtilis, and various species within the genera Pseudomonas, Streptomyces, and Staphylococcus. The constructs in host cells can be used in a conventional manner to produce the gene product encoded by the recombinant sequence. In some embodiments, introduction of the construct into the host cell can be accomplished by any suitable method known in the art (e.g., calcium phosphate transfection, DEAE-Dextran mediated transfection, or electroporation (e.g., Davis et al. (1986) Basic Methods in Molecular Biology). Alternatively, in some embodiments of the present invention, the polypeptides of the invention can be synthetically produced by conventional peptide synthesizers.

In some embodiments of the present invention, following transformation of a suitable host strain and growth of the host strain to an appropriate cell density, the selected promoter is induced by appropriate means (e.g., temperature shift or chemical induction), and the host cells are cultured for an additional period. In other embodiments of the present invention, the host cells are harvested (e.g., by centrifugation), disrupted by physical or chemical means, and the resulting crude extract retained for further purification. In still other embodiments of the present invention, microbial cells employed in expression of proteins can be disrupted by any convenient method, including freeze-thaw cycling, sonication, mechanical disruption, or use of cell lysing agents.

It is not necessary that a host organism be used for the expression of the nucleic acid constructs of the invention. For example, expression of the protein encoded by a nucleic acid construct may be achieved through the use of a cell-free in vitro transcription/translation system. An example of such a cell-free system is the commercially available TnT™ Coupled Reticulocyte Lysate System (Promega; this cell-free system is described in U.S. Pat. No. 5,324,637, hereby incorporated by reference).

4. Purification of ACSs

The present invention also provides methods for recovering and purifying ACSs from native and recombinant cell cultures including, but not limited to, ammonium sulfate precipitation, anion or cation exchange chromatography, phosphocellulose chromatography, hydrophobic interaction chromatography, affinity chromatography, hydroxylapatite chromatography and lectin chromatography. In other embodiments of the present invention, protein refolding steps can be used as necessary, in completing configuration of the mature protein. In still other embodiments of the present invention, high performance liquid chromatography (HPLC) can be employed as one or more purification steps.

In other embodiments of the present invention, the nucleic acid construct containing DNA encoding the wild-type or a variant ACS further comprises the addition of exogenous sequences (i.e., sequences not encoded by the ACS coding region) to either the 5′ or 3′ end of the ACS coding region to allow for ease in purification of the resulting polymerase protein (the resulting protein containing such an affinity tag is termed a “fusion protein”). Several commercially available expression vectors are available for attaching affinity tags (e.g., an exogenous sequence) to either the amino or carboxy-termini of a coding region. In general these affinity tags are short stretches of amino acids that do not alter the characteristics of the protein to be expressed (i.e., no change to enzymatic activities results).

For example, the pET expression system (Novagen) utilizes a vector containing the T7 promoter operably linked to a fusion protein with a short stretch of histidine residues at either end of the protein and a host cell that can be induced to express the T7 DNA polymerase (i.e., a DE3 host strain). The production of fusion proteins containing a histidine tract is not limited to the use of a particular expression vector and host strain. Several commercially available expression vectors and host strains can be used to express protein sequences as a fusion protein containing a histidine tract (e.g., the pQE series [pQE-8, 12, 16, 17, 18, 30, 31, 32, 40, 41, 42, 50, 51, 52, 60 and 70] of expression vectors (Qiagen) used with host strains M15[pREP4] [Qiagen] and SG13009[pREP4] [Qiagen]) can be used to express fusion proteins containing six histidine residues at the amino-terminus of the fusion protein). Additional expression systems which utilize other affinity tags are known to the art.

Once a suitable nucleic acid construct has been made, the ACS may be produced from the construct. The examples below and standard molecular biological teachings known in the art enable one to manipulate the construct by a variety of suitable methods. Once the desired ACS has been expressed, the enzyme may be tested for activity as described Examples 4 and 5.

5. Deletion Mutants of ACSs

The present invention further provides fragments of ACSs. In some embodiments of the present invention, when expression of a portion of an ACS is desired, it may be necessary to add a start codon (ATG) to the oligonucleotide fragment containing the desired sequence to be expressed. It is well known in the art that a methionine at the N-terminal position can be enzymatically cleaved by the use of the enzyme methionine aminopeptidase (MAP). MAP has been cloned from E. coli (Ben-Bassat et al. (1987) J. Bacteriol. 169:751-757) and S. typhimurium, and its in vitro activity has been demonstrated on recombinant proteins (Miller et al. (1990)PNAS 84:2718-1722). Therefore, removal of an N-terminal methionine, if desired, can be achieved either in vivo by expressing such recombinant polypeptides in a host producing MAP (e.g., E. coli or CM89 or S. cerevisiae), or in vitro by use of purified MAP. It is contemplated that deletion mutants of ACSs can be screened for activity as described above.

6. Use of ACS Nucleic Acids in Directed Evolution

It is contemplated that the ACS nucleic acids (e.g., SEQ ID NOs: 1-11, and fragments and variants thereof) can be utilized as starting nucleic acids for directed evolution. These techniques can be utilized to develop ACS variants having desirable properties such as increased synthetic activity or altered affinity for a particular fatty acid substrate.

In some embodiments, artificial evolution is performed by random mutagenesis (e.g., by utilizing error-prone PCR to introduce random mutations into a given coding sequence). This method requires that the frequency of mutation be finely tuned. As a general rule, beneficial mutations are rare, while deleterious mutations are common. This is because the combination of a deleterious mutation and a beneficial mutation often results in an inactive enzyme. The ideal number of base substitutions for targeted gene is usually between 1.5 and 5 (Moore and Arnold (1996) Nat. Biotech., 14, 458-67; Leung et al. (1998)Technique, 1:11-15; Eckert and Kunkel (1991) PCR Methods Appl., 1:17-24; Caldwell and Joyce (1992) PCR Methods Appl., 2:28-33; and Zhao and Arnold (1997) Nuc. Acids. Res., 25:1307-08). After mutagenesis, the resulting clones are selected for desirable activity (e.g., screened for ACS activity as described above). Successive rounds of mutagenesis and selection are often necessary to develop enzymes with desirable properties. It should be noted that only the useful mutations are carried over to the next round of mutagenesis.

In other embodiments of the present invention, the polynucleotides of the present invention are used in gene shuffling or sexual PCR procedures (e.g., Smith (1994) Nature, 370:324-25; U.S. Pat. Nos. 5,837,458; 5,830,721; 5,811,238; 5,733,731; all of which are herein incorporated by reference). Gene shuffling involves random fragmentation of several mutant DNAs followed by their reassembly by PCR into full length molecules. Examples of various gene shuffling procedures include, but are not limited to, assembly following DNAse treatment, the staggered extension process (STEP), and random priming in vitro recombination. In the DNAse mediated method, DNA segments isolated from a pool of positive mutants are cleaved into random fragments with DNAseI and subjected to multiple rounds of PCR with no added primer. The lengths of random fragments approach that of the uncleaved segment as the PCR cycles proceed, resulting in mutations in present in different clones becoming mixed and accumulating in some of the resulting sequences. Multiple cycles of selection and shuffling have led to the functional enhancement of several enzymes (Stemmer (1994) Nature, 370:398-91; Stemmer, (1994) Proc. Natl. Acad. Sci. USA, 91, 10747-51; Crameri et al. (1996) Nat. Biotech., 14:315-19; Zhang et al. (1997) Proc. Natl. Acad. Sci. USA, 94:4504-09; and Crameri et al. (1997) Nat. Biotech., 15:436-38). Variants produced by directed evolution can be screened for ACS activity by the methods described in Examples 4 and 5.

In further embodiments of the present invention, other combinatorial mutagenesis approaches are applied. For example, the amino acid sequences for a population of ACS homologs or other related proteins can be aligned, preferably to promote the highest homology possible. Such a population of variants can include, for example, ACS homologs from one or more species, or ACS homologs from the same species but which differ due to mutation. Amino acids appearing at each position of the aligned sequences are selected to create a degenerate set of combinatorial sequences.

In a preferred embodiment of the present invention, the combinatorial ACS library is produced by way of a degenerate library of genes encoding a library of polypeptides including at least a portion of potential ACS-protein sequences. For example, a mixture of synthetic oligonucleotides are enzymatically ligated into gene sequences such that the degenerate set of potential ACS sequences are expressible as individual polypeptides, or alternatively, as a set of larger fusion proteins (e.g., for phage display) containing the set of ACS sequences therein.

There are many ways in which the library of potential ACS homologs can be generated from a degenerate oligonucleotide sequence. In some embodiments, chemical synthesis of a degenerate gene sequence is carried out in an automatic DNA synthesizer, and the synthetic genes are ligated into an appropriate gene for expression. The purpose of a degenerate set of genes is to provide, in one mixture, all of the sequences encoding the desired set of potential ACS sequences. The synthesis of degenerate oligonucleotides is well known in the art (e.g., Narang, Tetrahedron 39:39, 1983; Itakura et al. (1981) Recombinant DNA, Proc 3rd Cleveland Sympos. Macromol., Walton, ed., Elsevier, Amsterdam, pp 273-289; Itakura et al. (1984) Annu. Rev. Biochem. 53:323; Itakura et al. (1984) Science 198:1056; and Ike et al. (1983) Nucleic Acid Res. 11:477). Such techniques have been employed in the directed evolution of other proteins (e.g., Scott et al. (1980) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815, each of which is incorporated herein by reference).

A wide range of techniques are known in the art for screening gene products of combinatorial libraries generated by point mutations, and for screening cDNA libraries for gene products having a particular property of interest. Such techniques are generally adaptable for rapid screening of gene libraries generated by the combinatorial mutagenesis of ACS homologs. The most widely used techniques for screening large gene libraries typically comprise cloning the gene library into replicable expression vectors, transforming appropriate cells with the resulting library of vectors, and expressing the combinatorial genes under conditions such that detection of a desired activity facilitates relatively easy isolation of the vector encoding the gene whose product was detected. The illustrative assays described below are amenable to high through-put analysis as necessary to screen large numbers of degenerate sequences created by combinatorial mutagenesis techniques.

In some embodiments of the present invention, the gene library is expressed as a fusion protein on the surface of a viral particle. For example, foreign peptide sequences can be expressed on the surface of infectious phage in the filamentous phage system, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of viral replication. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (e.g., WO 90/02909; WO 92/09690; Marks et al. (1992) J. Biol. Chem., 267:16007-16010; Griffths et al. (1993) EMBO J., 12:725-734; Clackson et al. (1991) Nature, 352:624-628; and Barbas et al. (1992) PNAS 89:4457-4461).

In another embodiment of the present invention, the recombinant phage antibody system (e.g., RPAS, Pharmacia Catalog number 27-9400-01) is modified for use in expressing and screening ACS combinatorial libraries. The pCANTAB 5 phagemid of the RPAS kit contains the gene encoding the phage gIII coat protein. In some embodiments of the present invention, the ACS combinatorial gene library is cloned into the phagemid adjacent to the gIII signal sequence such that it will be expressed as a gIII fusion protein. In other embodiments of the present invention, the phagemid is used to transform competent E. coli TG1 cells after ligation. In still other embodiments of the present invention, transformed cells are subsequently infected with M13KO7 helper phage to rescue the phagemid and its candidate ACS gene insert. The resulting recombinant phage contain phagemid DNA encoding a specific candidate ACS-protein and display one or more copies of the corresponding fusion coat protein. In some embodiments of the present invention, the phage-displayed candidate proteins that are capable of, for example, binding a particular acyl-CoA, are selected or enriched by panning. The bound phage is then isolated, and if the recombinant phage express at least one copy of the wild type gIII coat protein, they will retain their ability to infect E. coli. Thus, successive rounds of reinfection of E. coli and panning greatly enriches for ACS homologs, which are then screened for further biological activities.

In light of the present disclosure, other forms of mutagenesis generally applicable will be apparent to those skilled in the art in addition to the aforementioned rational mutagenesis based on conserved versus non-conserved residues. For example, ACS homologs can be generated and screened using, for example, alanine scanning mutagenesis, linker scanning mutagenesis, or saturation mutagenesis.

7. Chemical Synthesis of ACS Polypeptides

In an alternate embodiment of the invention, the coding sequence of an ACS is synthesized, whole or in part, using chemical methods well known in the art (e.g., Caruthers et al. (1980) Nuc. Acids Res. Symp. Ser., 7:215-233; Crea and Horn (1980) Nuc. Acids Res., 9:2331; Matteucci and Caruthers (1980) Tetrahedron Lett., 21:719; and Chow and Kempe (1981) Nuc. Acids Res., 9:2807-2817). In other embodiments of the present invention, the protein itself is produced using chemical methods to synthesize either a full-length ACS amino acid sequence or a portion thereof. For example, peptides can be synthesized by solid phase techniques, cleaved from the resin, and purified by preparative high performance liquid chromatography (e.g., Creighton, Proteins Structures and Molecular Principles, W H Freeman and Co, New York N.Y., 1983). In other embodiments of the present invention, the composition of the synthetic peptides is confirmed by amino acid analysis or sequencing (e.g., Creighton, supra).

Direct peptide synthesis can be performed using various solid-phase techniques (Roberge et al., Science 269:202-204, 1995) and automated synthesis may be achieved, for example, using ABI 431A Peptide Synthesizer (Perkin Elmer) in accordance with the instructions provided by the manufacturer. Additionally, the amino acid sequence of an ACS, or any part thereof, may be altered during direct synthesis and/or combined using chemical methods with other sequences to produce a variant polypeptide.

III. Identification of Other Acyl-CoA Synthetase Homologs

As described above, plant ACSs are members of a larger family of AMP-binding proteins (AMP-BPs). Therefore, methods for discriminating between AMP-BPs and true ACSs are desirable. FIG. 1 provides an amino acid comparison of the ACSs of the present invention (SEQ ID NOs:12-22) and ten putative Arabidopsis AMP-binding proteins (SEQ ID NOs: 33-42). The AMP-BP sequences were determined by BLAST searches of the TAIR database (The Arabidopsis Information Resource; http://www.arabidopsis.org/blast/) with ACS sequences. Most of the AMP-BP sequences were identified as BAC hits. The presumed cDNA sequences for these were deduced by homology comparisons to the ACSs and other AMP-BPs using GCG (Genetic Computer Group, Madison, Wis.). The sequences were then aligned using Pileup (Genetic Computer Group, Madison, Wis.) and shaded using the Boxshade server. The AMP-BP genes have also been isolated and sequenced, as described below (see Example 2).

This comparison led to the identification of at least nine conserved motifs in ACS, which are described in more detail below. Of these nine motifs, some are conserved between ACSs and AMBPS, while others are conserved only in ACSs; other motifs are conserved only in AMP-BPs, but these are not included in the nine motifs. The motifs are numbered from 1 to 9, in going from the amino to the carboxy terminal of the proteins. Where more than one amino acid occurs at a particular position in a motif, the most common amino acid is listed first, followed by less common amino acids, separated by a slash, which indicates that these amino acids occupy the same position in the motif. If more than four different amino acids occupy the same position, the position is indicated by an “X”, with the amino acids which occur at that position listed at the end of the sequence. Accordingly, in some embodiments, the present invention provides plant ACSs comprising at least one of ACS motifs 1-9, or nucleic acid sequences encoding such plant ACSs.

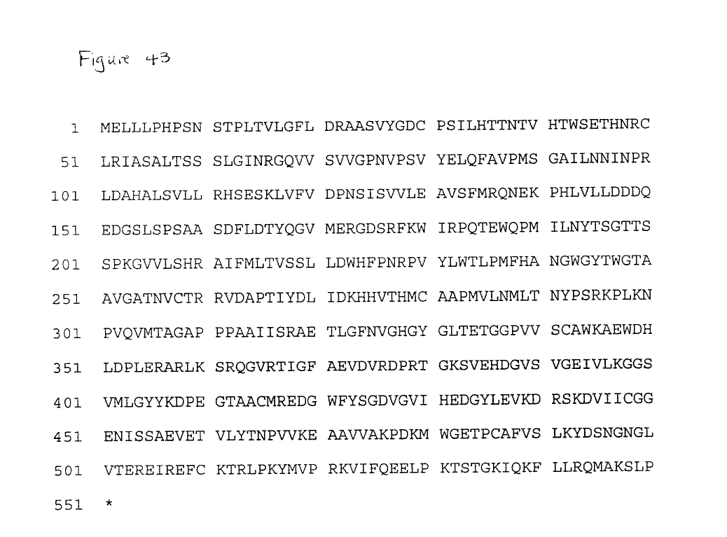

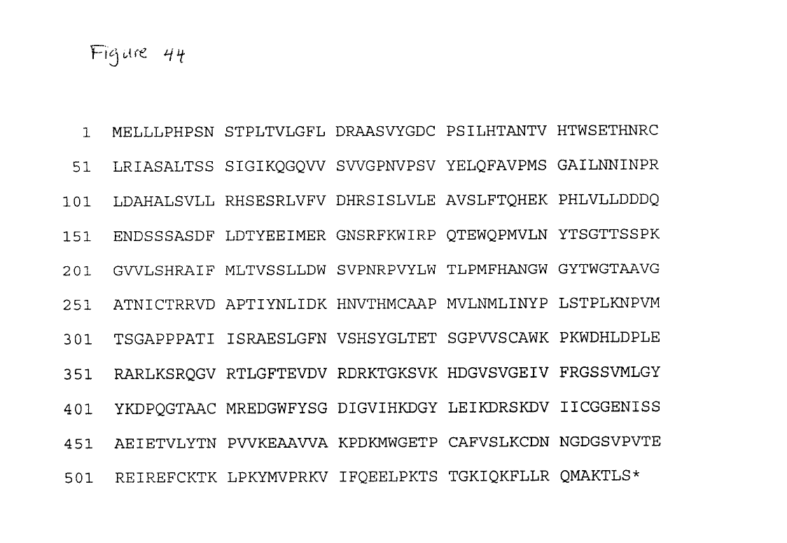

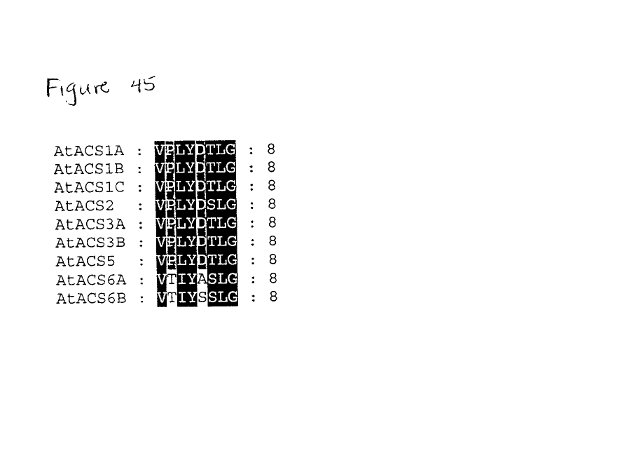

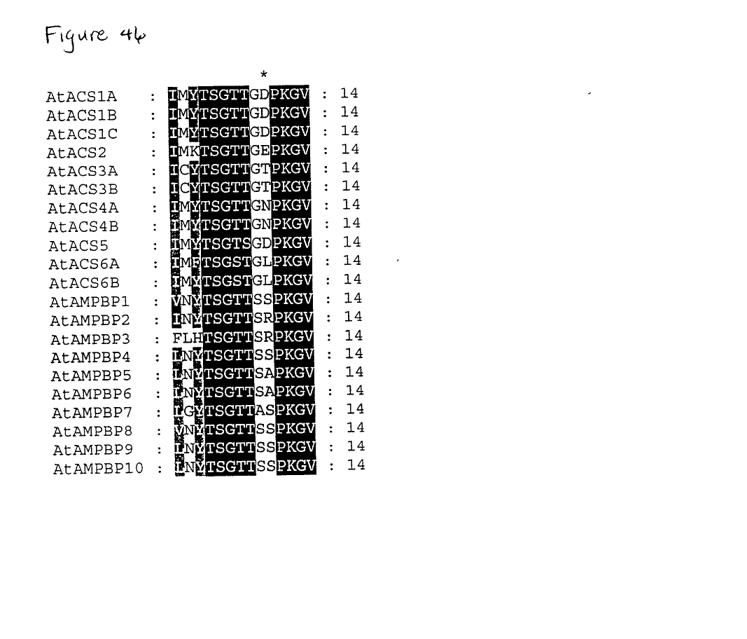

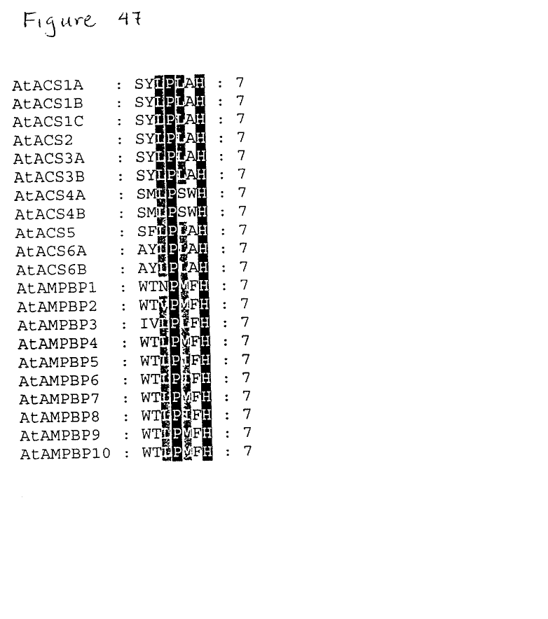

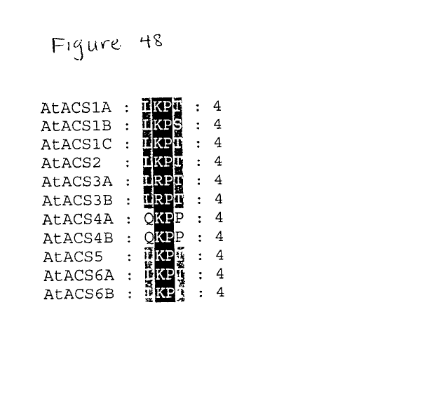

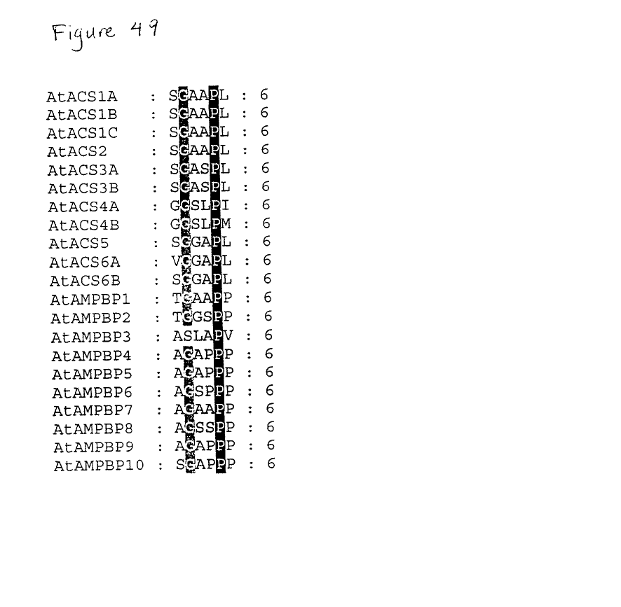

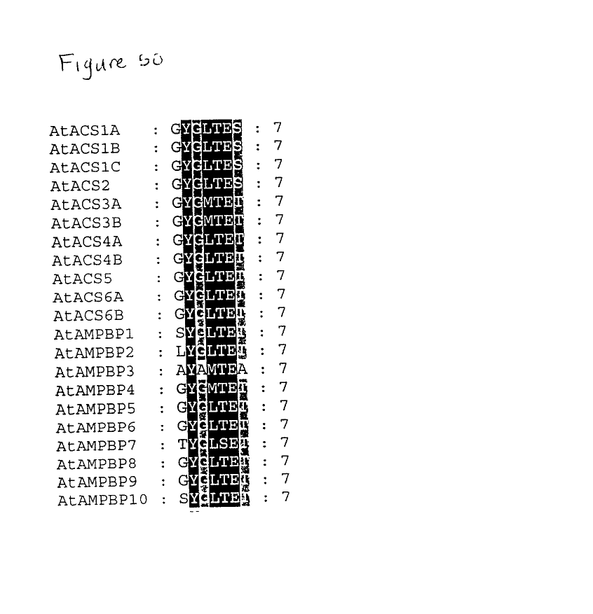

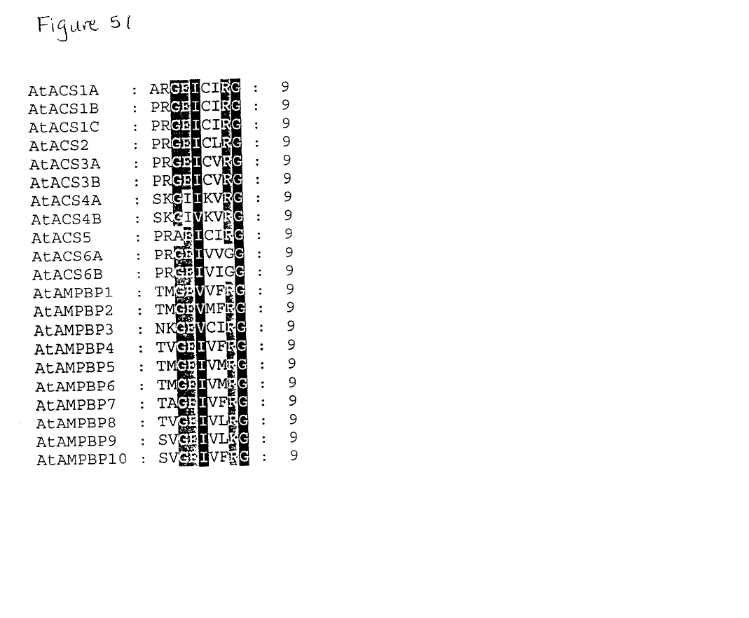

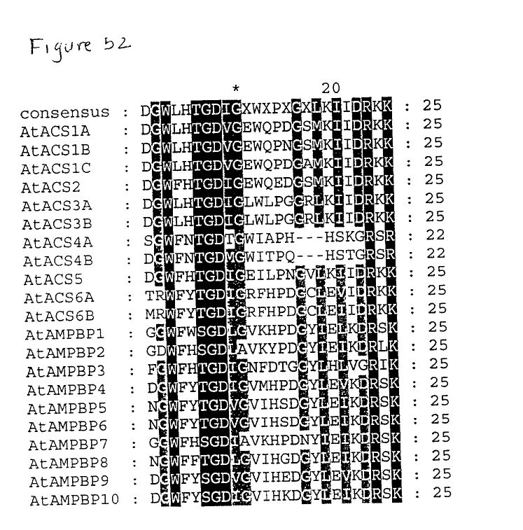

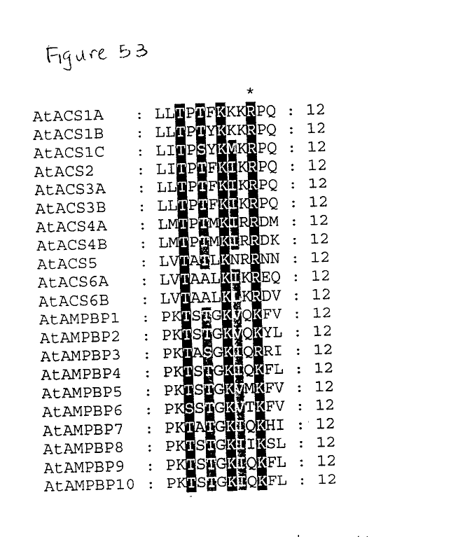

ACS motif 1 (FIG. 45; SEQ ID NO: 43, V-P/T-L/I-Y-D/A/S-T/S-L-G) is present in ACSs and absent in AMP-BPs. ACS motif 2 (FIG. 46; SEQ ID NO: 44, I-M/C-Y/F/K-T-S-G-T/S-T/S-G-X₁-P-K-G-V, where X₁ is D, L, T, N, or E) is conserved in both ACSs and AMP-BPs. The motif found in both ACSs and AMP-BPs is well known (PROSITE PS00455=[LIVMFY]−X2−[STG]−[STAG]−G−[ST]−[STEI]−[SG]−X−[PASLIVM]−[KR]), is very highly conserved, and acts as the unifying feature of the AMP-binding protein (AMP-BP) superfamily (Babbitt P C et al. (1992) Biochemistry 31(24): 5594-604; Fulda M et al. (1994) Mol Gen Genet 242(3): 241-9) to which ACS belongs. The sequence shown, SEQ ID NO: 44, is for ACSs alone, as the motif in ACSs differs slightly from that in AMP-BPs, particularly in amino positions 1, 2, 9, and 10. ACS motif3 (FIG. 47; SEQ ID NO:45, L-P-L/A-A-W-H) is present in ACSs and absent in AMP-BPs. ACS motif 4 (FIG. 48; SEQ ID NO:46; L/Q-K-P-T/P) is present in ACSs and absent in AMP-BPs. ACS motif 5 (FIG. 47; SEQ ID NO: 49, S/G/V-G-A/G/S-A/L/A-P-L/T/M) is present in ACSs and absent in AMP-BPs. ACS motif 6 (FIG. 50; SEQ ID NO: 48, G-Y-G-L-T-E-T/S/A) is present in both ACSs and AMP-BPs. Note that only G occupies the first position in ACSs, while several different amino acids occupy this position in AMP-BPs. ACS motif 7 (FIG. 51; SEQ ID NO: 49, P/S/A-R/K-G/A-E/I-I-C/K/V-V/I-R/G-G) is present in ACSs and is absent in AMP-BPs. ACS motif 8 (FIG. 52; SEQ ID NO:50, I-I-D-R-K-K) is present in ACSs, except AtACS4A and AtACS4B, and absent in AMP-BPs. The 25 amino acid consensus sequence shown at the top of FIG. 52 is a consensus sequence derived from several genes (for example, from E. coli, yeast, and human) which are known to bind fatty acids; this 25 amino acid sequence is implicated in fatty acid binding in E. coli genes, based upon experiments in which mutagenesis of 15 of the 25 amino acids resulted in absent or different specificity fatty acid binding (Black, P N (1997) J Biol Chem 272: 4896-4903). ACS motif 9 (FIG. 53; SEQ ID NO: 51, L-L/V/M-P/A-T/A/S-F/L/M/Y-K-I/K/M/L-K/R-R) is present in ACSs and absent in AMP-BPs.

It is contemplated that the sequences described herein can be utilized to clone and characterize ACS homologs from other species of plants. Accordingly, in some embodiments, the ACS nucleic acids or fragments thereof are utilized to screen cDNA or genomic libraries prepared from the RNA or DNA of another plant species. In other embodiments, primers that are completely or partially complementary to portions of SEQ ID NOs:1-11 are utilized to amplify ACS homologs from nucleic acid isolated from other plant species. For example, degenerate primers may be utilized to amplify ACS homologs for genomic DNA samples or cDNA samples from other species. Alternatively, RT-PCR may be utilized to directly amplify homologs from RNA isolated from other species.

It is also contemplated that the sequences described herein (e.g., both nucleic acid and polypeptide sequences, SEQ ID NOs: 1-22), may be utilized to search computer databases for homologous sequences from other species. For example, BLAST searches (Altshul et al. (1997) Nucleic Acids Res. 25:3389-3402; http://www.ncbi.nlm.nih.gov/blast) may be utilized to search for nucleic acids and proteins having homology (e.g., greater than 60%, 70%, 80%, or 90%) to SEQ ID NOs:1-22.

In some embodiments, nucleic acids suspected of being ACS homologs are screened by comparing motifs. In some embodiments, the protein sequence can be analyzed for the presence or absence of one or more of ACS motifs 1-9 (SEQ ID NOs: 43-51, respectively). The presence or absence of these motifs indicates that the candidate ACS is a true ACS. In still further embodiments, the nucleic acids can be utilized in genetic screens for ACS activity. For example, the nucleic acids can be analyzed for complementation of the mutant S. cerevisiae strain YB525. In other embodiments, the nucleic acids can be expressed and analyzed for complementation or biochemical activity as described in Example 4 and 5.

Within the ACS group, AtACS4A and AtACS4B are somewhat divergent from the other ACS genes. This conclusion is based upon the observation that in motifs 3, 4, 5, and7, the amino acids for AtACS4A and AtACS4B are likely to be different from those of the other ACSs, yet these different amino acids are generally identical to each other in AtACS4A and AtACS4B. This conclusion is also supported by the observation that AtACS4A and AtACS4B do not contain motif 8. Moreover, this conclusion is also supported by the inability to observe ACS enzyme activity, either by complementation or by an in vitro assay, with these two clones (see Examples 4 and 5). Yet these two genes are more closely related to the ACSs than to any of the other genes in the superfamily. It is possible that these genes encode ACSs that activate specialized substrates, or are inactive under the conditions used in these experiments due to special requirements, such as folding or multimer formation requirements, or the need for post-translational modifications not met by the cellular machinery of Saccharomyces cerevisiae. Alternatively, these genes may encode a different type of enzyme related to ACS. For example, in yet another possibility, it is contemplated that these two enzymes are acyl-ACP synthetases. This function can be examined by over-expressing the ACS4A and ACS4B in yeast, and then assaying yeast extract for acyl ACP synthase activity, in a manner similar to that described in Examples 4 and 5, in which ACP is used as a substrate instead of CoA.

IV. AMP Binding Proteins

A construction of the phylogenetic relationship between all 44 members of the Arabidopsis AMP-BP superfamily (Shockey, J et al., manuscript in preparation) revealed several interesting phenomenon. Only three genes (At3gl6170, At3g48990, and At1g30520) align independently, while the other 41 members of the superfamily separate into three main groups: The ACS subfamily; a subfamily containing the three known 4-coumarate-CoA ligases plus ten other related genes; and a subfamily of fourteen previously unknown genes.

The discovery of the third subfamily was unexpected. This subfamily as a whole was more closely related than the other two groups, containing at least 42% amino acid identity, while bearing weak and roughly equal homology (approximately 20-25% amino acid identity) to the ACS, acetyl-CoA synthetase, and 4-coumarate-CoA ligase genes. Searches of all public databases revealed that higher plants (including rice and Brassica sp.) are the only organisms that contain genes highly homologous to those of this third subfamily. This subfamily thus represents a unique class of enzymes that may play a specialized role in a plant-specific aspect of carboxylic acid activation. It is also possible that this subfamily represents a functionally equivalent but structurally unrelated counterpart to the ACS subfamily.

In order to characterize this subfamily of genes, full-length cDNAs for ten of the fourteen members of this subfamily were cloned into pYES2 and transformed into Saccharomyces cerevisiae YB525, as described in the following examples (see particularly Examples 1 and 4 and 5). These constructs were used in the complementation and in vitro enzyme activity analyses, exactly as described for the ACS genes in the following examples. In the complementation assays, the genes of the AtAMP-BP subfamily were unable to activate exogenous myristic acid, and all ten genes were therefore unable to complement the YB525 phenotype. In the in vitro enzyme assays, cell-free lysates prepared from these transformed yeast lines containing one of these ten genes were also inactive against oleic acid in the in vitro enzyme assays.

These data do not rule out the possibility that the genes of this group are ACSs. In fact, the phylogenetic analysis of the AMP-BP superfamily as a whole supports the hypothesis that these genes catalyze the coenzyme A-dependent activation of some type of carboxylic acid, given the fact that each of the other classes in the phylogenetic tree contain representative genes that do exactly that. It is contemplated that AMP-BPs are very long chain ACSs. Medium chain- or very long chain-CoA synthetases have been characterized in other organisms ((Min and Benzer, 1999)). While medium-chain fatty acids are very rare in Arabidopsis ((Ohlrogge and Browse, 1995)), a critical role for very long chain acyl groups is obvious. Very long chain fatty acids (>C24) are the substrates for the biosynthesis of the complex mixture of esters, alcohols, ketones, aldehydes, and alkanes that make the cuticular wax layer present on the surface of plants. Cuticular waxes also play essential roles in plant fertility and insect defense ((Preuss, D et al. (1993) Genes Dev 7(6): 974-85). This function can be examined by over-expressing the AMP-BPs in yeast, and then assaying yeast extract for very long chain ACS activity, in a manner similar to that described in Examples 4 and 5.

### EXAMPLES

The following examples serve to illustrate certain preferred embodiments and aspects of the present invention and are not to be construed as limiting the scope thereof.

In the disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade); ATP (adenosine 5′-monophosphate); BSA (bovine serum albumin); cDNA (copy or complimentary DNA); CS (calf serum); DNA (deoxyribonucleic acid); ssDNA (single stranded DNA); dsDNA (double stranded DNA); DNTP (deoxyribonucleotide triphosphate); LH (luteinizing hormone); NIH (National Institutes of Health, Besthesda, Md.); RNA (ribonucleic acid); PBS phosphate buffered saline); g (gravity); OD (optical density); HEPES (N-[2-Hydroxyethyl]piperazine-N-[2-ethanesulfonic acid]); SDS (sodium dodecylsulfate); Tris-HCl (tris[Hydroxymethyl]aminomethane-hydrochloride); rpm (revolutions per minute); EDTA (ethylenediaminetetracetic acid); bla (β-lactamase or ampicillin-resistance gene); ORI (plasmid origin of replication); and Sigma (Sigma Chemical Company, St. Louis, Mo.); GC (gas chromatography); fames (fatty acid methyl esters).

### Example 1

This Example describes the procedures utilized to identify and clone the ACS genes of the present invention.

Sequencing and Homology Analysis

All DNA sequencing was conducted in the Macromolecular Analysis Laboratory at Washington State University using automated sequencing equipment (Applied Biosystems, Foster City, Calif.). Sequences were assembled and modified using the GCG suite of programs (Wisconsin Package Version 10.0, Genetics Computer Group, Madison, Wis.). Database searches were conducted against the AtDB Illustra database (genome-www.standford.edu/Arabidopsis), its successor at The Arabidopsis Information Resource (TAIR) (www.arabidopsis.org), and the Munich Information Center for Protein Sequences Arabidopsis thaliana database (MATDB) (mips.gsf.de/proj/thal/db/search/search_frame.html).

Identification and cloning of genes

Full-length ACS clones were isolated by first screening the EST databases ((Newman et al. (1994) Plant Physiol 106(4): 1241-55) to identify partial cDNA clones with homology to known ACSs. The inserts from these clones were used to screen for full length clones present in any of various cDNA libraries available from the Arabidopsis Biological Resource Center ((Weigel, D et al. (1992) Cell 69(5): 843-59; and Kieber, J J et al. (1993) Cell 72(3): 427-41). When full-length clones could not be identified using this approach, the missing portions of the genes were identified by isolation of genomic clones from an Arabidopsis thaliana genomic DNA library ((Voytas, D F et al. (990) Genetics 126(3): 713-21).

Once the initiator codon of each gene had been determined, a new gene-specific oligonucleotide primer pair was used to amplify RT-PCR products spanning the full-length open reading frame. Briefly, 2 ug of total RNA from mature seeds, tissue-culture-grown roots, stems, young rosette leaves, flowers, and siliques were used as template for a scaled-up first-strand cDNA synthesis, using an equimolar mixture of capped oligo-dT primers (T₂₀C, T₂₀A, and T₂₀G) and Superscript II reverse transcriptase as described in the Hieroglyph differential display manual (Genomyx Corp.). Aliquots of these reactions were used as template in amplifications using Pfu Turbo polymerase (Stratagene, La Jolla, Calif.), or with ExTaq polymerase (PanVera, Madison, Wis.), as described in the respective manufacturer's protocol. The Pfu Turbo-generated products were cloned into the pCR-ScriptCam vector supplied in the blunt cloning kit (Stratagene). The ExTaq-generated products were cloned into the pCR2.1 vector supplied in the TOPO-TA cloning kit (Invitrogen). These clones were sequenced to verify the fidelity of amplification.

Cloning of Arabidopsis ACS genes in E. coli and Saccharomyces cerevisiae

For expression in yeast, one of two methods was used to reamplify the open reading frames of the Arabidopsis cDNAs for re-cloning. Some genes were amplified from the original plasmids using new oligonucleotide primer pairs that introduced restriction sites compatible for insertion into the multiple cloning site of the Saccharomyces cerevisiae inducible expression vector pYES2 (Invitrogen). The PCR products were restricted with appropriate enzymes then gel-purified. Concentrated solutions of the insert DNAs were ligated to appropriately digested pYES2 DNA and transformed into competent E. coli. Plasmid DNA from the resulting bacterial colonies was resequenced to ensure accurate reamplification then transformed into S. cerevisiae YB525 cells (provided by Prof. J I Gordon, Washington University, St. Louis, Mo.) ((Knoll, L J et al. (1995) Genetics 126(3): 713-21) that had been made competent for chemical transformation using the S. c. EasyComp kit (Invitrogen). Alternatively, PCR products for some of the ACS cDNAs were generated using the sticky end PCR technique ((Zeng, G (1998) Biotechniques 25(2): 206-8). These products were ligated, transformed, and sequenced as described above.

Acyl CoA Synthetases

AtACS1A

The cDNA clone corresponding to 240K22T7 was ordered from ABRC and unsuccessfully used to screen the Lambda PRL2 cDNA library. The remaining sequence was determined by isolation of a genomic clone from the genomic library using 240K22T7 insert as probe. The full-length cDNA was amplified using the new sequence information and cloned into a pPCR-Script Amp vector (Stratagene) and sequenced. Due to problems encountered when recloning this construct, the cDNA was reamplified from pooled RT reactions. The primers used for this amplification added KpnI and SphI sites to the 5′ and 3′ ends of the gene, respectively. The resulting PCR product was then cut with these two enzymes and cloned into the same sites in the yeast expression vector pYES2 (Invitrogen) and sequenced.

AtACS1B

AtACS9 was found by searching the AGI database for sequences homologous to AtACS 7 and 8. Primers were designed based on the putative start and stop codons. The primers successfully amplified an appropriately sized product from genomic DNA, but to date attempts to RT-PCR a cDNA clone have been unsuccessful. The genomic product itself has not yet been cloned.

AtACS1C

The cDNA clone corresponding to 119E14T7 and unsuccessfully used to screen the Lambda PRL2 cDNA library. The remaining sequence was determined by isolation of a genomic clone from a genomic library using the 119E14T7 insert as probe. The sequence determined from the genomic clone was used to design primers for amplification of the full-length cDNA from DNA prepared from the cDNA libraries. This cDNA was cloned into pYES2 in a fashion similar to that described for AtACS7.

AtACS2

The cDNA clone corresponding to EST 229E14T7 was ordered from ABRC. The insert DNA was excised and used as probe for screening the Lambda PRL2 cDNA library. A clone was isolated with an approximately 2 kb insert and excised from the plasmid DNA. Sequencing revealed that the 5′ end of the cDNA was missing based on homology to Brassica sequences. Five prime RACE amplifications were performed with total phage DNA isolated from the cDNA library. This led to the cloning and sequencing of the 5′ sequence.

AtACS3A

The cDNA clone corresponding to EST 205M6T7 from ABRC and used to isolate an apparently full length clone from the Lambda PRL2 cDNA library.

AtACS3B

The cDNA clone corresponding to EST 123N12T7 was ordered from ABRC and unsuccessfully used to screen the Lambda PRL2 cDNA library. The remaining sequence was determined by isolation of a genomic clone from the genomic library using the 123N12T7 insert as probe. The full-length cDNA was amplified using the new sequence information, cloned into the pPCR-Script Cam vector (Stratagene), and sequenced.

AtACS4A

This gene, originally named AMP-BP3 and later renamed AtACS4A, was identified from the Arabidopsis databases using the sequence of the Brassica AMP-BP clone pMF28P (Genbank Accession # Z72151). The presumed start codon and stop codon were identified by homology. The full-length cDNA was amplified by RT-PCR using the primers AMP-BP35SacICut (5′-TGCATGGAGCTCATGGCTTCGACTTCTTCTTTGGGAC-3′) (SEQ ID NO: 73) and AMP-BP33XhoICut (5′-ACGATCCTCGAGTTAACTGTAGAGTTGATCAATCTC-3′) (SEQ ID NO: 74). The resulting PCR product was cut with SacI and XhoI and ligated into the same sites in the yeast expression vector pYES2 (Invitrogen) and sequenced.

AtACS4B

The presence of this gene was found in the Arabidopsis database by homology to AtACS4A. The start and stop codons were deduced and primers designed according to them. The primers 4B-KpnI (5′-CGAATGGTACCAATGGCTTCAACGTCTCTCGGAGCTTCG-3′) (SEQ ID NO: 75) and 4B-3SphI (5′-ATACTGCATGCCTACTTGTAGAGTCTTTCTATTTCA-3′) (SEQ ID NO: 76) were used to amplify the full-length cDNA by RT-PCR. The resulting PCR product was cloned directly into the blunt-end vector pCRScript-Cam (Stratagene) and sequenced. The insert was cut using KpnI and SphI. Unfortunately, this cut the gene into two pieces. The 5′ Kpn-Sph fragment was cloned into pYES2 first. The resulting construct was cut with SphI and the 3′ Sph-Sph fragment of AtACS4B was ligated into it.

AtACS5

The cDNA clone corresponding to EST GbGe115a was ordered from ABRC. The insert DNA was excised and used as probe for screening the Lambda PRL2 cDNA library. A clone was isolated and again found to be missing sequence from the 5′ end of the OR, which was determined by 5′ RACE. The full-length cDNA was cloned into pPCR-Script Cam vector (Stratagene) and sequenced.

AtACS6A

The cDNA clone corresponding to EST G2B10T7 from ABRC and used to isolate an apparently full length clone from the Lambda PRL2 cDNA library.

AtACS6B

The cDNA clone corresponding to EST 203J11T7 was ordered from ABRC. The insert DNA was excised and used as probe for screening the Lambda PRL2 cDNA library. A almost full-length clone was isolated. Sequence missing from the 5′ end of the open reading frame was determined by isolating a genomic clone from a genomic DNA library (ABRC) using the 203J11T7 insert as a probe. The full-length cDNA open reading frame was amplified with new primers designed from sequence from the 3′ end of the partial cDNA clone and the 5′ sequence of genomic clone. The cDNA was cloned into pPCR-Script Cam vector (Stratagene) and sequenced.

### Example 2

This Example describes the cloning of ten AMP-BPs. These ten AMP-BPs were selected from a total of fourteen members of AMP-BPs discovered through the grouping of the original 44 genes into subfamilies as determined by phylogenetic relationships among the 44 genes (Shockey, J et al. (2001), manuscript in preparation), as described above. The methods of sequencing and homology analysis, identification and cloning of genes, and cloning of Arabidopsis genes in E. coli and Saccharomyces cerevisiae are described in Example 1, with additional details provided below.

Total RNA was isolated from Arabidopsis dry seeds, roots, old stems, young stems, young leaves, old leaves, young stems, old stems, flowers, new siliques, and old siliques. First strand cDNA was prepared from each of these RNA preps with Superscript II reverse transcriptase (Gibco-BRL) as described in the Hieroglyph mRNA Profile Kit (Genomyx). Using gene specific primers designed from the expected start codon and stop codon of each gene (Example 3), the open reading frame for each gene was amplified from a pool of all of the RT reaction.

The PCR reactions were carried out on an MJ Research PTC 100 thermal cycler. The polymerase was ExTAQ (Panvera Corp.). The reactions (50 μl) contained 5 μl of the 10×Taq buffer, 4 μl of the 10 mM dNTP mix (Panvera) 5 μl each of 5 μM stocks of the 5′ and 3′ primers and 2 μl of the pooled RT reactions. The conditions were: 95° C. for 3 minutes, followed by 30 cycles of 95° C. for 20 sec, 58° C. for 30 sec, 72° C. for 1 minute. A final 72° C. incubation of 2 minutes was followed by an indefinite 4° C. hold until samples were removed.

A small amount of each reaction was analyzed by agarose gel electrophoresis to ascertain successful amplification. The remainder of each successful amplification was electrophoresed and the band cut out followed by purification of the DNA from the gel slice using Qiagen gel extraction columns. A 4 μl aliquot of each DNA was ligated to TOPO-activated pCR2.1 vector (Invitrogen), using their standard conditions and transformed into TOP10F' competent cells supplied with the kit. Positive transformants were selected by growth on agar plates containing either 100 (g/ml carbenicillin or 50 μg/ml kanamycin plus X-GAL and IPTG for blue/white screening. Colonies containing plasmids with AMP-BP inserts were identified by colony PCR screening several white colonies, using the same PCR conditions as described above. Representative positive colonies for each gene were grown in 50 ml of liquid L-broth plus appropriate antibiotic overnight at 37° C., followed by isolation of plasmid DNA using Promega's Wizard MidiPrep kit.

Plasmid DNA was quantified spectrophotometrically and sequenced with several vector- and gene-specific primers.

AtAMP-BP1

The full length gene was isolated from 2-3 Kb size-selected cDNA library (Kieber et al. (1993) Cell 72(3): 427-441) obtained from the Arabidopsis Biological Resource Center (ABRC) at Ohio State University. The insert from the partial cDNA clone 99N9T7 (Genbank Accession # T22607) was used as the probe. After sequencing, the full-length open reading frame was amplified from this plasmid with Pfu Turbo Polymerase (Stratagene) with primers containing restriction sites compatible for cloning into the yeast expression vector pYES2 (Invitrogen). The product was cut out and ligated into pYES2 using standard procedures.

AtAMP-BP3

The cDNA clone corresponding to EST FAFM13 was ordered from the Arabidopsis Biological Resource Center (ABRC, Ohio State University). The insert DNA was excised and used as probe for screening a Lambda PRL2 cDNA library (also obtained from the ARBC). A clone was identified and isolated. The insert DNA from the lambda phage clone was excised by in vivo excision as described in library instructions resulting in the gene fused in pBlueScript SK+.

All other AMP-BPs

All other AMP-BP genes were cloned by identification in the databases by homology to cloned Arabidopsis ACS genes. The start codon and stop codon were identified and primers designed to these spots. These primers may or may not have contained restriction sites to facilitate cloning. The full-length open reading frames were amplified by RT-PCR from total RNA. These PCR reactions were carried out with one of two different DNA polymerases: ExTaq (Panvera) or Pfu Turbo (Stratagene). Those products (AtAMP-BPs 2, 4, 5, 6, and 7) generated with ExTaq were cloned directly into the A-overhand vector pCR2.1 (Invitrogen). These genes were later cut out of pCR2.1 and ligated into pYES2. The Pfu Turbo generated AtAMP-BP8 product was cloned into the blunt-end vector pCRScript-CAM (Stratagene), then cut out of this vector and ligated into pYES2. The Pfu Turbo products for AtAMP-BP9 and 10 were cut with KpnI and SphI and cloned directly into pYES2.

### Example 3

This Example describes primers useful for amplifying full-length ACSs and AMP-BPs and for use in RNAse protection assays.

| | |
|---|---|
| AtACS1A | |
| (SEQ ID NO: 52) | AAGGCGATTCATCTTGAC - AtACS1A gene specific RPA primer |
| (SEQ ID NO: 53) | CTGGTACCATGACGCAGCAGAAGAAATAC-5′ yeast vector cloning primer + KpnI |
| | restriction site. |
| (SEQ ID NO: 54) | CTCTCGAGCTACCCTCTGGAAGCAAATT |
| AtACS1B | |
| (SEQ ID NO: 55) | ATGACGTCGCAGAAAAGATTCATCTTTG-5′ start codon cloning primer |
| (SEQ ID NO: 56) | TTACTGTCCGGAAGCTAGACTTTCCTTTC-3′ stop codon cloning primer |
| AtACS1C | |
| (SEQ ID NO: 57) | GAGTCTATCTGCCGAAACC-AtACS1C gene specific RPA primer |
| (SEQ ID NO: 58) | ATGGCGACTGGTCGATACATCGTTGAGGTTG-5′ start codon cloning primer |
| (SEQ ID NO: 59) | TTACACTCGTAGCTGCACTTCTC-3′ stop codon cloning primer |
| AtACS2 | |
| (SEQ ID NO: 60) | 6RPA-AACTCAATTACCAATCTCCC |
| (SEQ ID NO: 61) | CGCCATGAACACCGAGTCAG-5′ Start codon cloning primer |
| (SEQ ID NO: 62) | GAGCCATTCAGAGCTTCGACG-3′ Stop codon cloning primer |
| AtACS3A | |
| (SEQ ID NO: 63) | ATCCGAGAGTGAAAGCAG-AtACS3A gene specific RPA primer |
| (SEQ ID NO: 64) | CTGGTACCATGGATTCTTCTTCTTCGTC-5′ start codon for cloning into yeast |
| | expression vector pYES2, KpnI restriction site included. |
| (SEQ ID NO: 65) | AGCTCGAGTTCACAAACCTCTATTAGCAG-3′ stop codon for cloning into |
| | pYES2, XhoI restriction site included. |
| AtACS3B | |
| (SEQ ID NO: 66) | CTTGCTGAGATGGATGAC-AtACS3B gene specific RPA primer |
| (SEQ ID NO: 67) | CATGGAATTTGCTTCGCCGGAAC |
| (SEQ ID NO: 68) | GTACCATGGAATTTGCTTCGCCGGAAC -5′ KpnI overhang sticky-end primers |
| | for cloning into yeast expression vector pYES2 (Invitrogen). |
| (SEQ ID NO: 69) | CTCACAGTTTAGAAGGAATGGGG |
| (SEQ ID NO: 70) | CATGCTCACAGTTTAGAAGGAATGGGG-3′ SphI overhang sticky end cloning |
| | primers for cloning into pYES2. |
| AtACS4A | |
| (SEQ ID NO: 71) | ATGGCTTCGACTTCTTCTTTGGGA |
| (SEQ ID NO: 72) | CAAATGTCTTAACTGTAGAGTTGATCA |
| (SEQ ID NO: 73) | TGCATGGAGCTCATGGCTTCGACTTCTTCTTTGGGAC AMP-BP35SacICut |
| (SEQ ID NO: 74) | ACGATCCTCGAGTTAACTGTAGAGTTGATCAATCTC-3′) AMP-BP33XhoICut |
| AtACS4B | |
| (SEQ ID NO: 75) | CGAATGGTACCAATGGCTTCAACGTCTCTCGGAGCTTCG-4B-KpnI |
| (SEQ ID NO: 76) | ATACTGCATGCCTACTTGTAGAGTCTTTCTATTTCA-4B-3SphI |
| AtACS5 | |
| (SEQ ID NO: 77) | ACGGCAGAAAAGAACAAG-AtACS5 gene specific RPA primer |
| (SEQ ID NO: 78) | CTGGTACCATGAAGTCTTTTGCGGCTAAG-5′ start codon primer for cloning |
| | into pYES2, KpnI restriction site included. |
| (SEQ ID NO: 79) | ACTCTAGATTATTGATACATATAACGTAC-3′ stop codon primer for cloning |
| | into pYES2, XbaI restriction site included. |
| AtACS6A | |
| (SEQ ID NO: 80) | ATGGAAGATTCTGGAGTGAATCCAATG-5′ start codon cloning primer |
| (SEQ ID NO: 81) | TTAGGCATATAACTTGCTGAGTTCATC-3′ stop codon cloning primer |
| AtACS6B | |
| (SEQ ID NO: 82) | CTTCAAAGCAAGGAATAGAC-AtACS6B gene specific RLPA primer |
| (SEQ ID NO: 83) | ATGATTCCTTATGCTGCTGGTG-AtACS6B 5′ Start codon cloning primer |
| (SEQ ID NO: 84) | TTAGGCATATAACTTGGTGAGATC-3′ stop codon cloning primer |
| AtAMP-BP1 | |
| (SEQ ID NO: 85) | ATGGAGGGAACTATCAAATCTC-5′ start codon cloning primer |
| (SEQ ID NO: 86) | TCATAACTTGCTTCTGCCTTTC-3′ stop codon cloning primer |
| AtAMP-BP2 | |
| (SEQ ID NO: 87) | ATGAGATTCT TGTTAACCAA AAG -5′ start codon cloning primer |
| (SEQ ID NO: 88) | TTACAAGCTA CCCATTTCAT CAG-3′ stop codon cloning primer |
| AtAMP-BP3 | |
| (SEQ ID NO: 89) | TGAGAAATATGGGGAAGAG - AtAAMP-BP gene specific RPA primer |
| (SEQ ID NO: 90) | ATGGATAGCGATACTCTCTCAG-5′ Start codon cloning primer |
| (SEQ ID NO: 91) | TCAGGGCTTCTCAAGGAAATG-3′ Stop codon cloning primer |
| AtAMP-BP4 | |
| (SEQ ID NO: 92) | ATGGAACTTT TACTCCCACA CG-5′ start codon cloning primer |
| (SEQ ID NO: 93) | TCATCAAGGCAAGGACTTAG C-3′ stop codon cloning primer |
| AtAMP-BP5 | |
| (SEQ ID NO: 94) | GAAAACAATACATTGACCACTCAAGATG-5′ gene specific cloning primer |
| (SEQ ID NO: 95) | TCGCAAGTTCTAATTTTACATCCGACTC-3′ gene specific cloning primer. |

AMP-BP5 and AMP-BP6 are very similar, therefore the gene-specific cloning primers were moved “outward” from the start and stop codons a bit, to ensure gene-specificity.

| | |
|---|---|
| AtAMP-BP6 | |
| (SEQ ID NO: 96) | TTTGATTACCACTAGGAGGAAGAGATG-5′ gene specific cloning primer |
| (SEQ ID NO: 97) | CGGTGAAAGAAAGACGTTTAAGAAATTG-3′ gene specific cloning primer |
| AtAMP-BP7 | |
| (SEQ ID NO: 98) | ATGGCGGCAACGAAGTGGCGTG-5′ start codon cloning primer |
| (SEQ ID NO: 99) | CTATAACCTGCTTCTTGGTACTGGTCCC-3′ stop codon cloning primer |
| AtAMP-BP8 | |
| (SEQ ID NO: 100) | ATGGAAGATTTGAAGCCAAG TGCC-5′ start codon cloning primer |
| (SEQ ID NO: 101) | TTACATGTTTTTGGCAATCT CTTTAAGC-3′ stop codon cloning primer |
| AtAMP-BP9 | |
| (SEQ ID NO: 102) | TACAAAACATTAACAAAAATCAAAGTATGG |
| (SEQ ID NO: 103) | ATAACTCAAGCGAATCTTTAAGGCAGAGA |
| AtAMP-BP10 | |
| (SEQ ID NO: 104) | ACGATACTATAGTTTCTTGCAGCTAACTAA |
| (SEQ ID NO: 105) | TTATTTAATGGACTTGTTCAAGACAGGGT |

AMP-BP9 and 10 are so similar that primers upstream of the start codon and downstream of the stop codon had to be used to ensure gene-specific amplifications.

### Example 4

This Example describes the detection of ACS enzyme activity by complementation. Eleven candidate ACS genes were cloned into the galactose-inducible Saccharomyces cerevisiae expression vector pYES2. These constructs were tested for their ability to complement the phenotype of Saccharomyces cerevisiae strain YB525. This yeast strain contains insertional disruptions in two of its ACS genes, FAA1 and FAA4 ((Knoll, L J et al. (1995) J Biol Chem 270(18): 10861-7), which are responsible for the majority of ACS enzyme activity in S. cerevisiae. Thus, these cells are completely dependent on complementation by an active ACS when grown on media containing fatty acids as a sole carbon source and cerulenin to inhibit endogenous fatty acid synthesis by the fatty acid synthase complex.

A culture of YB525 was grown in YBD liquid media until approximately mid-log phase. Cells were harvested and made competent for transformation using the S.c. EasyComp kit (Invitrogen). Arabidopsis cDNAs were ligated into the pYES2 vector (Invitrogen), then checked for proper orientation and sequence. Any base pairs that did not match the AGI database sequence were corrected using the Quickchange site-directed mutagenesis kit (Stratagene). The expression constructs were transformed into chemically competent YB525 cells and uracil auxotrophs selected on DOBA-ura plates (DOBA: 2% yeast nitrogen base, 2% dextrose, 0.1% complete supplement mixture lacking uracil, 17 g/L agar) (BIO101). Representative colonies were chosen at random and grown until mid- to late-log phase in DOB liquid media (DOBA minus agar). Galactose was added to a concentration of 2% to induce high-level expression of the transgenes from the GAL1 promoter of the vector. The cultures were then grown for an additional 2 to 4 hours. Aliquots of each culture were diluted 1:1 (vol/vol) with 2 M sorbitol and 5 ul aliquots plated on DOBA plates containing galactose plus 500 uM myristic acid and 25 uM cerulenin, followed by incubation at 30° C. for 3-4 days.

The results of the complementation experiment show that after four days at 30° C., seven of the eleven candidate ACS genes had complemented the mutant phenotype and restored growth rates to wild-type levels, as compared to the wild-type strain Invisc (Invitrogen) that was used as a positive control. Only AtACS3A, 3B, 4A, and 4B did not complement the mutant phenotype.

The explanation for the inability of some of the genes to restore cerulenin-insensitive growth to this strain was obvious. The AtACS3A and AtACS3B genes contain PTS2 and PTS1 peroxisome targeting sequences, respectively. Targeting of an ACS to the peroxisome renders the enzyme inaccessible to the pool of exogenous fatty acid, as evidenced by the inability of Faa2p, the endogenous peroxisomal Saccharomyces ACS ((Johnson, D R et al. (1994) J Cell Biol 127(3): 751-62; and Knoll, L J et al. (1995) J Biol Chem 270(18): 10861-7), to support growth under the conditions used in this experiment.

The inability of the AtACS4A and AtACS4B genes to complement the YB525 strain was less easily explained. The deduced amino acid sequences for these two proteins did not contain recognizable peroxisome targeting sequences. AtACS4A and 4B do contain N-terminal extensions, however, that may target the encoded enzymes to other sites within the yeast cell that are separated from the pool of exogenous fatty acids. These two genes also contain abnormally long insertional elements, as seen in FIG. 2. This difference in length was also observed in bnapmf28, the Brassica napus homolog of AtACS4A, which was also inactive in ACS assays when over-expressed in E. coli ((Fulda, M et al. (1997) Plant Mol Biol 33(5): 911-22).

In general, the results of the complementation experiment indicate that most of the candidate genes are in fact ACSs, and that the insertional element described above is a reliable tool for distinguishing ACS genes from other related AMP-binding protein genes.

### Example 5

This Example describes a biochemical assay for ACS activity. The results of the yeast complementation experiment clearly demonstrated that many of the candidate genes chosen from the initial library screens and database searches did encode ACS enzymes. However, additional analysis was necessary to address the inability of the AtACS3A, 3B, 4A, and 4B genes to complement the ACS deficiency in the S. cerevisiae YB525. In order to directly test the ability of this family of genes to produce active ACS enzymes, cell-free lysates were prepared from S. cerevisiae YB525 cells over-expressing each of the eleven candidate ACS genes, as described below. These lysates served as enzyme sources in ACS enzyme activity assays, using ¹⁴C-labeled oleic acid as a substrate.

Enzyme overproduction in Saccharomyces cerevisiae

Transformed YB525 cells were selected on solid selective media lacking uracil. Several colonies from each transformation were restreaked on a new selective media plate. Representative colonies were randomly chosen to inoculate liquid media cultures. This media lacked uracil and contained dextrose as the carbon source, which suppressed the GAL1 promoter of the pYES2 vector. These cultures were grown at 30° C. with vigorous shaking to an optical density at 600 nm of about 0.7-1.0. Galactose (20% w/v) was added to a final concentration of 2% to induce gene expression. The cultures were shaken at 30° C. for an additional 2-4 hours and the cells harvested by centrifugation. The yeast cells were washed once with distilled water and harvested again for spheroplast production. Spheroplasts were generated from intact cells using lytic enzyme (ICN) following the manufacturers protocol. The spheroplasts were lysed by sonication on ice (2×1 min) followed by removal of solid debris by centrifugation at 8,000×g for 15 min at 4° C. The resulting supernatants were used as enzyme sources for the ACS assay.

ACS enzyme assay

The assay conditions were similar to those described previously (Fulda, M et al. (1997) Plant Mol Biol 33(5): 911-22. The assay was conducted in 1.5 ml Eppendorf tubes in a volume of 100 ul. The assay mixture contained 100 mM Bis-Tris-propane (pH 7.6), 10 mM MgCl₂, 5 mM ATP, 2.5 mM dithiothreitol, 1 mM CoA, 10 uM 1-¹⁴C-labeled oleic acid (specific activity 50-57 mCi/mmol, DuPont-NEN), and 20 ug of crude yeast cell lysate protein. The assay was initiated by addition of the fatty acid and incubated at room temperature for 15 minutes. The reactions were stopped by addition of 100 ul of 10% acetic acid in isopropanol and extracted twice with 900 ul of hexane (previously saturated with 50% isopropanol). Enzyme activity was measured by analyzing aliquots of the aqueous phase by liquid scintillation counting. Lysates from yeast cells bearing the empty pYES2 vector served as a negative control, while commercial ACS enzyme from Pseudomonas sp. (Sigma) served as the positive control.

Results

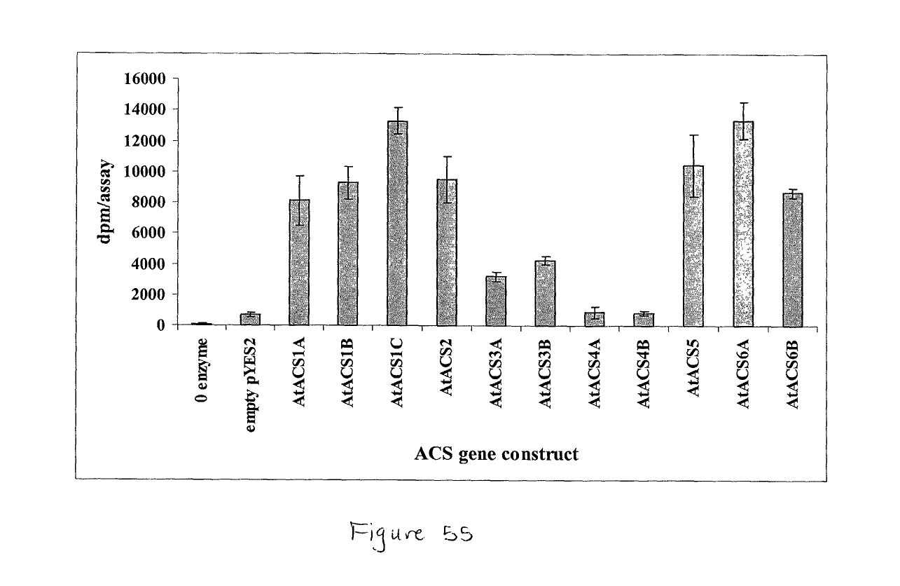

The results of these assays are shown in FIG. 55, and demonstrate that all cell lines except those containing the AtACS4A and AtACS4B constructs produced significant levels of ACS activity. The results for these two genes was consistent with those observed in the yeast complementation experiment and in the E. coli expression studies ((Fulda, M et al. (1997) Plant Mol Biol 33(5): 911-22). Thus, in contrast to the complementation study, cells containing constructs AtACS3A and AtACS3B produced active enzymes. The levels of activity produced by these two constructs was somewhat lower than that produced by the other active genes; thus, the activity of AtACS3A and 3B was approximately 5-6-fold higher than that of the empty pYES2 negative control, compared to 12- and 20-fold higher activity for AtACS1A and AtACS6A, respectively. These levels of activity demonstrate that the AtACS3A and AtACS3B genes encode ACS. These results also further demonstrate that the other seven members of this family are ACSs as well.

The lack of enzyme activity for cells containing AtACS4A and 4B constructs provide further support to the hypothesis that the enzymes encoded by these genes are unique with respect to the other nine ACS genes. These genes may encode ACSs that activate specialized substrates, or the may encode a different type of enzyme related to ACS. It is also possible that these enzymes are indeed ACSs, but are inactive under the conditions used in these experiments due to special folding or multimer formation requirements, or the need for post-translational modifications not met by the cellular machinery of Saccharomyces cerevisiae.

Alternatively, it is contemplated that these two genes encode acyl ACP synthetases, as described previously.

### Example 6

This Example describes the cellular location of ACS transcription as assayed by RNAse protection assays and by RNA expression profiles.

RNAse protection assays

In vitro transcription and RNAse protection assays were performed basically as described in the Maxiscript and RPA II manuals (Ambion), respectively. Briefly, several different tissues (e.g., seed, cultured roots, stem, young leaves [post-bolting], silique, flowers and buds, green rosette [pre-bolting], and older leaves [post-bolting]) were harvested from wild-type Arabidopsis ecotype Columbia plants. Tissues were frozen in liquid nitrogen and stored at −80° C. until use.

Total RNA was isolated from the tissues using standard methods. The RNA pellets were dissolved in DEPC-treated water and quantified spectrophotometrically. Gene specific RPA probes templates were produced by PCR amplifying small (200-500 bp) fragments of each ACS gene from the full-length or partial cDNA clones obtained from ABRC. Primer sequences are provided in Example 3. The PCR products were electrophoresed through TAE-agarose gels and gel-purified using Qiaquick spin columns (Qiagen).

The PCR products were transcribed in vitro in 20 μl reactions containing: 2 μl 10×transcription buffer, approximately 1 μg of template DNA, 1 μl each ATP, CTP, and GTP, 5 μl 12.5 μM ³²P labeled UTP, and 2 μl either SP6, T3, or T7 RNA polymerase. The contents were mixed and incubated at 37° C. for 1 hour. DNAse I was added to stop the reaction and remove template DNA.

The radiolabeled RNA probe was then gel-purified on 5% TBE, 8 M Urea acrylamide gels. The RNA was eluted in elution buffer (0.5 M ammonium acetate, 1 mM EDTA, 0.1% SDS) overnight. An aliquot of the eluted probe was quantified by scintillation counting and, according to the manufacturer's calculation methods, the number of counts corresponding to 2 femptomoles of probe was determined. Twenty micrograms of total RNA from each tissue was co-precipitated with 2 femptomoles of probe and resuspended in 20 μl hybridization buffer (Solution A from the kit). After heating at 95° C. for 3-4 minutes, the RNA/probe mixture was incubated overnight at 45° C.

Unprotected RNA was digested by adding to the RNA/probe mixture 200 ml RNAse solution (1/100 dilution of stock RNAse A/RNAse Ti mixture) and incubating the mix at 37° C. for 30 minutes. Three hundred microliters of solution Dx was then added to each tube to stop the reaction. Two microliters of carrier yeast RNA was added to increase pellet visibility. The mixture was chilled at −20° C. for at least 15 minutes, and then centrifuged at maximum speed for minutes in a cold room. The pellets were dissolved in nondenaturing gel sample buffer and electrophoresed through a nondenaturing 5% TBE acrylamide gel. After running, the gel was dried in a gel drier and the images were developed in a Bio-Rad Phosphorimager.

The results are summarized in Table 2 below. A relatively strong signal for a given tissue is designated by (+++), a relatively weak signal is designated by (+), and the apparent absence of a signal is indicated by (−). As can be seen, the RNAs for the different ACSs localize to a variety of tissues.

TABLE 2
RNAse Protection Assay Results

| | Tissue | | | | | | | |
|---|---|---|---|---|---|---|---|---|
| | dry, mature | cultured | | young | | flowers and | green | older |
| ACS | seed | roots | stem | leaves | silique | buds | rosette | leaves |
| AtACS1A | − | ++ | + | + | − | +++ | + | − |
| AtACS1C | − | + | + | − | + | + | + | + |
| AtACS2 | − | +++ | + | ++ | + | ++ | +++ | + |
| AtACS3A | + | − | + | + | − | + | na | na |
| AtACS3B | ++ | + | + | + | − | ++ | na | na |
| AtACS5 | + | − | + | − | − | +++ | na | na |
| AtACS6B | − | ++ | − | − | − | +++ | na | na |

RNA expression profiles

The tissue-specific RNA expression profiles of each of the ACS genes was also examined by semi-quantitative RT-PCR ((Kong, S E et al. (1999) Anal Biochem 271(1): 111-4). This technique was chosen because careful control of the PCR conditions allows for easy and sensitive comparisons of the expression levels for each of the different genes while eliminating the risk of cross-hybridization between related genes on a Northern blot. Each gene was analyzed using RNA from mature seeds, tissue culture-grown roots, leaves, stems, flowers, and siliques.

RNA preparations from mature seed, roots, young leaves, stems, siliques, and flowers were quantified spectrophotometrically and 1 ug aliquots of each used as template for reverse transcription, as described above. One ul of each RT reaction was used as template in a 50 ul PCR reaction containing gene-specific primers. The amplification conditions were as follows: 95° C. 3 min, and 30 cycles of 94° C. 15 sec, 55° C. 30 sec, 72° C. 1 min. One-third of each reaction was analyzed by TAE-agarose gel electrophoresis and the degree of gene expression correlated to the relative intensity of each band as determined by visual comparison of the ethidium bromide staining intensity when the gels were visualized under UV illumination. The actin gene ACT8 ((An et al., 1996)) was used as a control to insure that equal amounts of RNA were used in both the RT and PCR portions of the experiments.

The results are summarized in Table 3 below. The relative strength of the signal is scored from 3 plusses (“+++”), denoting the strongest signal, to a negative sign (“−”), denoting the apparent absence of a signal.

TABLE 3
Tissue Specific RNA Expression Assay Results

| | Tissue | | | | | |
|---|---|---|---|---|---|---|
| | dry, | | | | | |
| | mature | cultured | | | | |
| ACS | seed | roots | stem | Leaves | flowers | siliques |
| AtACS1A | − | ++ | + | + | ++ | + |
| AtACS1B | − | − | − | − | ++ | − |
| AtACS1C | − | + | − | + | + | + |
| AtACS2 | − | + | ++ | + | +++ | − |
| AtACS3A | ++ | ++ | + | + | ++ | + |
| AtACS3B | + | + | − | + | ++ | − |
| AtACS4A | ++ | ++ | ++ | + | +++ | + |
| AtACS4B | +++ | + | + | + | ++ | − |
| AtACS5 | − | ++ | + | + | ++ | + |
| AtACS6A | ++ | + | + | + | ++ | + |
| AtACS6B | + | + | − | + | ++ | + |

The relative intensities of the bands for the positive control, the Arabidopsis actin ACT8 gene, were almost equivalent, with slight reductions in mature seed and siliques. This profile closely parallels the relative Northern blot signal intensities for this gene ((An, Y Q et al. (1996) Anal Biochem 271(1): 111-4), thus validating the accuracy of this technique. As seen in Table 3, most of the ACS genes are expressed in a variety of tissues at widely varying levels.

Close inspection of Table 3 reveals several interesting phenomena. First, several ACS genes are expressed in the mature seed of the plant. The deposition of transcripts for these genes in the mature seed indicates that the ACS enzymes encoded by them are needed during the very early stages of germination. This is consistent with a strong demand for the enzymes of beta-oxidation and membrane lipid biosynthesis in the emerging seedling. The second interesting pattern observed is the strength of expression of all eleven ACS genes in flowers. These data are consistent with the high level of metabolic activity in flowers. The overall complexity of expression for the genes in this group suggests that at least some of the ACSs may have overlapping functions within the plant. Only AtACS1B seems to be highly specific, showing extremely high expression in flowers, but no expression in any of the other tissues tested. Nearly all the ACS genes, with the exception of AtACS1B and possibly AtACS2, are expressed in siliques.

In other experiments, the RNA expression pattern of AtACS6A (the closest paralog of AtACS6B) is similar to 6B in that highest levels of expression were observed in young, developing leaves and seeds; this is consistent with the belief that de novo FAS is most active in these tissues. This observation suggests that many genes in this gene family may participate in glycerolipid synthesis in the developing seed.

### Example 7

This Example describes the analysis of the subcellular localization of ACSs by a chloroplast import assay. Briefly, intact chloroplasts were isolated from young pea seedling extracts by centrifugation through Percoll gradients, and incubated with labeled expression products from an in vitro transcription/translation reaction mixture with an ACS encoding sequence. The chloroplasts were then separated from the labeled expression products by centrifugation through a Percoll cushion, lysed, and the different fractions of the chloroplast separated. The import of the labeled ACS was determined by the presence of label in chloroplast lysates, the location was determined by the presence of label in different fractions, and the identification of labeled ACS was confirmed by gel electrophoresis.

Chloroplasts are isolated from nine to ten day old pea seedlings by first removing the seedlings from a growth chamber and placing them in lab light for at least one hour to allow for starch degradation before grinding the tissue (this minimizes disruption of intact chloroplasts).

Next, a standard Percoll gradient was formed by adding 1 mg glutathione to a 50 ml open top centrifuge tubes, followed by the addition of 17.5 ml 2×GR buffer (1×GR buffer is 50 mM HEPES/KOH pH 8.0, 10 mM EDTA, 0.33 M sorbitol, 5 mM Na⁺ ascorbate, pH 7.5, and 0.05% BSA) and 17.5 ml Percoll. The mixture was then covered with parafilm and mixed. Next, the tubes were centrifuged in SS34 rotors at 4° C. min at 19,000 rpm (no brake).

When the gradient was almost complete, the aerial portions of the plants were cut and placed in a pre-weighed flask (about 40 g of tissue from a flat planted with ˜200 ml peas). The tissue was placed in a chilled blender containing 250 ml 1×GR and pulsed three times for one second each. The extract was filtered through a funnel lined with cheesecloth and Miracloth. The process was then repeated with a second 40 g batch. The pooled extracts were placed in chilled 250 ml bottles and pelleted in a swinging bucket rotor for 3 min at 3200 rpm. The supernatant was decanted, and the pellet resuspended in 5 ml 1×GR. The pellets (containing chloroplasts) were then layered onto the gradients with a glass pipette and centrifuged in a swinging bucket rotor at 2600 rpm for 15 min. The lower intact chloroplast band was removed and placed into two 50 ml tubes. The tubes were filled to top with 1×IB (1×IB buffer is 50 mM HEPES/KOH, pH 8.0, 0.33 M sorbitol) and centrifuged in a swinging bucket rotor at 2600 rpm for 5 min. The supernatant was removed and the pellet resuspended in 10 ml of IB.

The concentration of chloroplasts was determined by placing 1 ml acetone in each of three 1.5 ml tubes. Water (250 μl) was added to the first tube, 225 μl water and 25 μl chloroplasts were added to the second tube, and 200 μl water and 50 μl chloroplasts were added to the third tube. The tubes were mixed well and centrifuged to pellet the proteins. The OD at 652 mn was determined and the concentration of chloroplasts calculated by the following formula: (OD652/34.5)×1.25)/sample amount×10 ml=mg total. The chloroplasts samples were then repelleted and resuspended to 1 mg/ml in 1×IB.

Labeled ACS gene products were prepared by in vitro transcription and translation of ACS cDNAs using a TNT kit (Promega) according to the manufacturer's instructions. Labeled control proteins for the import assay were also prepared in the same manner; these control proteins included luciferase, which is not imported into chloroplasts, the small subunit of RiBisCO, which is imported and is localized to the stroma, with concomitant cleavage of the signal peptide (Froelich, J E et al. (2001) Plant Physiol 125: 306-317), and LeHPL, a tomato hydroperoxide lyase which is associated with the chloroplast envelopes, despite its lack of a typical signal peptide (Froelich, J E et al. (2001) Plant Physiol 125: 306-317).

Import assays were performed in following reaction mixtures: 75 μl 1×IB, 5 μl 2×IB, 15 μl 50 mM Mg-ATP (in IB), 50 μl 2×chloroplasts (1 mg/ml), and 5 μl translation product. The reaction mixtures were incubated in water bath at 25° C. for 15-30 min in the presence of light. The import reaction mixtures were then loaded onto 1 ml of 40% Percoll and centrifuged at 3,000×g for 8 min. The supernatant was removed, the pellet resuspended, and centrifuged again. Next, 600 μl lysis buffer (25 mM HEPES+5 mM MgCl₂) was added to the pellet. This mixture was incubated on ice, in the dark, for about 20 min. The mixture was then divided into 3 equal parts in microfuge tubes and centrifuged in an Airfuge at 100,000×g for 40 min at 4° C. The pellets were then resuspended in either 200 μl lysis buffer, 200 μl 2M NaCl, or 100 mM Na₂CO₃. The mixtures were then centrifuged in an Airfuge at 100,000×g for 30 min at 4° C. The supernatant was removed and 100% TCA added to 10%. The mixtures were stored overnight.

The next day, the mixtures were centrifuged at 20,000×g for 10 min, washed with cold acetone, and resuspended in 30 μl 5×SDS Loading dye. Ten microliters of the chloroplast import assays were then loaded onto 10% nondenaturing gels and electrophoresed. Following electrophoresis, the gels were dried and exposed to film.

The results indicate that despite the lack of a typical chloroplast targeting signal, labeled AtACS6B was targeted to intact chloroplast, and was only present in the membrane fractions. Treatment of the lysed membranes with lysis buffer and NaCl did not dissociate AtACS6B from the membranes, whereas treatment with Na₂CO₃ extracted a portion of it from the membranes. This pattern was similar to that observed with a control protein, LeHPL, a hydroperoxide lyase from tomato which has been shown to associate with chloroplast outer envelope, even though it too lacks a signal peptide (Froelich, J E et al. (2001) Plant Physiol 125: 306-317). Thus, the results suggest that AtACS6B is associated with the chloroplast envelope membranes. Moreover, ATACS6B does not appear to be proteolytically processed during plastidial targeting, because the gel mobility of the AtACS6B associated with the chloroplast was identical to that of the starting product, produced by in vitro translation.

Additional results indicated that AtACS2 is also imported into chloroplasts.

### Example 8

This Example describes identification and analyses of ACS knock-out mutant Arabidopsis plants. Two different mutants were found in two different lines of T-DNA Arabidopsis plants.

The first population, a T-DNA tagged population, available through the Arabidopsis Biological Resource Center (http://aims.cps.msu.edu/aims/), represents 6,000 individual transformants, each containing one or more T-DNA insertions. The T-DNA is a 17.0 kb DNA fragment that contains the nptII gene, which confers resistance to kanamycin. Insertions of the large T-DNA fragment in a gene of interest effectively prevents transcription of that gene.

This population was searched using a P1/KFLB primer combination (primers listed below), and resulted in the identification of a mutant line in the CD5-7 population (Feldmann lines) that contains a T-DNA interrupted AtACS6B coding region. The T-DNA insertional event occurs in the third exon, 1120 bp downstream from the start codon in the genomic sequence. From a sample of pooled seeds, two mutants were identified by using P1/KFLB and the P1/P2 gene specific primer combinations in PCR analysis first on pooled and later on individual plants: a heterozygous mutant containing one copy of a T-DNA interrupted AtACS6B gene, and a homozygous mutant lacking both native copies of AtACS6B (both designated the T₁ generation). The seeds were germinated after surface sterilization in 20% bleach+0.1% SDS for 20 minutes, followed by rinsing 3 times in sterile water. The sterilized seeds suspended in 0.1% agarose were plated on germination medium (MS salts, 1% sucrose, 3.5 g/L Phytagel, 75 mg/L kanamycin, pH 5.7). PCR analysis and protocols were performed according to the protocols at http://www.biotech.wisc.edu/Arabidopsis/ using PanVera ExTaq.

| | | |
|---|---|---|
| P1 primer | (GAAAGTTAAACTCAATTCCTCCGTCGATCA) | (SEQ ID NO: 106) |
| P2 primer | (GCATATAACTTGGTGAGATCTTCAGAGAATT) | (SEQ ID NO: 107) |
| KFLB primer | (TGCACTCGAAATCAGCCAATTTTAGACAA). | (SEQ ID NO: 108) |

In order to screen for the presence of multiple T-DNA insertions, progeny from the heterozygous T₁ plants were subjected to segregation analysis. The kanamycin segregation ratios of the T₂ seed of the heterozygous mutant indicated that only one T-DNA insertional event was present. Of 471 seed, 121 were kanamycin-sensitive, while 370 were resistant to kanamycin. This ratio represents a 3:1 hypothesis for a single insertion (x²=0.033; P>0.8). Southern blot analysis of 5 T₂ plants from homozygous mutant showed identical restriction patterns to the heterozygous plants when probed with a LB fragment, confirming that the homozygous T₁ individual also contained only one insert.

Results from a Northern blot analysis showed the lack of full-length AtACS6B transcript in the acs6b/acs6b mutant. Total RNA was isolated from floral and bud tissues of wild type, heterozygous, and homozygous AtACS6B plants. As expected, transcripts of full-length AtACS6B were present only in wild-type and heterozygous mutant plants. A truncated transcript corresponding to the length of transcript preceding the T-DNA insertion was present in the heterozygous and homozygous mutants.

A comparison of the phenotypes of the homozygous mutant and the wild-type plants showed that at all stages of the life cycle, the homozygous mutant was indistinguishable from wild type plants grown under the same conditions. Quantitative measurements of growth rate also showed no difference between the homozygous mutant and wild-type plants.

Fatty acid analysis of above-ground portions of wild type and homozygous mutant plants at 19 days of age revealed no significant differences between any of the fatty acid species typically found in Arabidopsis leaves (fatty acids were analyzed as methyl esters of total extracted lipids).

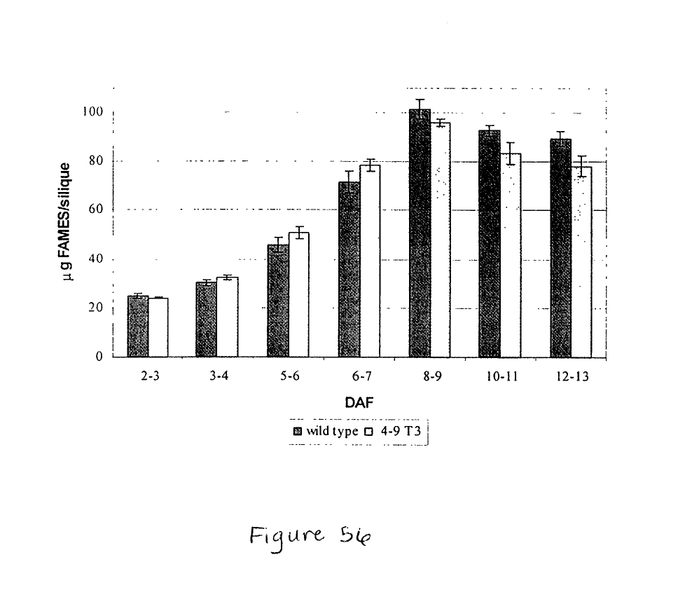

Northern analysis showed that the AtACS6B transcript was more abundant in developing seeds than in leaves. Therefore, lipids of developing seeds from homozygous and wild-type plants were analyzed. The plants were grown under 14 hour photoperiod, and secondary and axillary floral stems were removed as they appeared in order to facilitate the cataloging and collection of siliques. At 42 days, intact siliques of varying developmental stages were removed and the total fatty acids analyzed. The lipid content of the homozygous mutant from 2 to 13 DAF did not differ significantly from that of wild type plants (see FIG. 56). The peak of lipid accumulation (8-9 days after flowering, or DAF) corresponds to the highest level of AtACS6B transcripts at 6 to 11 DAF developing siliques.

ACS activity was measured in chloroplasts isolated from wild type and homozygous mutant plants. Intact chloroplasts were isolated from 19 day old leaf tissue as described in Example 7. ACS was assayed as described in Example 6; the assay included isolated chloroplasts, CoenzymeA, ATP, and 1-¹⁴C-oleic acid (18:1). When compared with wild type, the homozygous mutant chloroplasts exhibited a 13.75-fold decrease in ACS activity in this assay.

In summary, these results indicate that in the AtACS6B knock-out mutant, there were no visible phenotypic differences or measurable changes in fatty acid quantity or species between wild type and homozygous mutant plants, yet the homozygous mutant chloroplasts exhibited significantly less ACS activity than did the wild-type plants.

Another mutant, an ACS2 T-DNA knockout mutant, was also discovered, but in a different population of T-DNA mutant plants. This population of T-DNA mutant plants was prepared in a glabrous plant line, which is a Columbia mutant which is missing the gene responsible for developing trichomes. Thus, the wild-type plant for this mutant is a glabrous plant, or one which does not have trichomes.

The phenotype of the ACS2 mutant is quite different from that of the wild-type, in that the mutant has smaller, curled leaves and flowers slightly later. Segregation analysis indicated that the homozygous ACS2 knockout plant (11-4) contained multiple T-DNA insertions. To obtain a plant line which contained only insertions in the ACS2 genes, the plants were backcrossed with Columbia pollen. After several generations of selfing, plant lines which contained only insertions (homozygous) in ACS2 were obtained. These plants exhibited the small, puckered leaf phenotype of the original mutant, indicating that the absence of functional ACS2 transcript was responsible for the phenotype. On the other hand, even though phenotypically this mutant is quite different, the leaf fatty acids of this mutant do not appear to differ significantly from those of the wild-type plant.

Leaf fatty acids were analyzed by removing leaves from each of a wild-type plant (glabrous, “glb”), progeny of the original mutant plant with the same phenotype (homozygous, “11-4”), and progeny of the original mutant plant crossed with wild-type phenotype which exhibits a wild type phenotype (which is therefore believed to be hemizygous, “wt”), and placing them in individual glass screw-cap tubes. One and a half milliliters 2.5% H₂SO₄ in methanol were added to each tube and the tubes were incubated at 80° C. for 1.5 hours. Next, 1.5 ml water and 500 μl hexane were added to each tube. The tubes were vortexed and centrifuged to separate the phases. The hexane phases were then transferred to GC vials for GC analysis according to the following program: 150° C. for 1 min, then ramp at 15 degrees/min to 240° C., then hold for 2 min.

The fatty acid profiles of the mutants did not differ significantly from those of wild-type plants (See Table 4).

TABLE 4
Fatty acid profiles of leaves obtained from wild-type plants (“glb”; five
different leaves from one plant were analyzed), progeny of the original
ACS2 mutant plant crossed with the same phenotype (homozygous,
“11-4”; five different plants were analyzed), and progeny
of the original mutant ACS2 plant with wild-type phenotype
(hemizygous, “wt”; five different plants were analyzed).

| Fatty | | | | | | | | | |
|---|---|---|---|---|---|---|---|---|---|
| acid | 16:0 | 16:1c | 16:1t | 16:2 | 16:3 | 18:0 | 18:1 | 18:2 | 18:3 |
| Re- | 4.39 | | 4.69 | 4.80 | 5.19 | 5.51 | 5.64 | 5.92 | 6.29 |
| ten- | | | | | | | | | |
| tion | | | | | | | | | |
| time | | | | | | | | | |
| glb- | 11.76 | | 1.33 | 0.36 | 10.99 | 0.88 | 0.88 | 9.18 | 41.41 |
| #1 | | | | | | | | | |
| glb- | 13.72 | | 3.23 | 0.69 | 12.49 | 0.94 | 1.20 | 9.88 | 41.68 |
| #1 | | | | | | | | | |
| glb- | 13.50 | 0.63 | 2.54 | 0.41 | 11.27 | 1.18 | 1.53 | 10.27 | 44.84 |
| #1 | | | | | | | | | |
| glb- | 12.51 | 0.36 | 2.85 | 0.39 | 11.36 | 0.71 | 0.74 | 9.09 | 45.62 |
| #1 | | | | | | | | | |
| glb- | 13.47 | | 2.81 | 0.41 | 11.52 | 0.95 | 0.82 | 9.69 | 48.62 |
| #1 | | | | | | | | | |
| Aver- | 12.99 | 0.50 | 2.55 | 0.45 | 11.53 | 0.93 | 1.03 | 9.62 | 44.43 |
| age | | | | | | | | | |
| 11-4 | 12.18 | 0.52 | 2.28 | 0.62 | 11.48 | | 1.62 | 12.15 | 40.69 |
| #1 | | | | | | | | | |
| 11-4 | 11.82 | 0.47 | 2.15 | 0.62 | 10.96 | 0.96 | 2.34 | 13.14 | 36.96 |
| #2 | | | | | | | | | |
| 11-4 | 11.83 | 0.63 | 2.47 | 0.86 | 11.90 | 0.54 | 1.82 | 10.23 | 41.17 |
| #3 | | | | | | | | | |
| 11-4 | 12.74 | 0.57 | 2.13 | 0.62 | 12.14 | | 2.21 | 13.01 | 40.60 |
| #4 | | | | | | | | | |
| 11-4 | 12.20 | 0.49 | 1.99 | 0.54 | 11.10 | 0.59 | 1.66 | 12.79 | 41.27 |
| #5 | | | | | | | | | |
| Aver- | 12.15 | 0.54 | 2.20 | 0.65 | 11.52 | 0.70 | 1.93 | 12.26 | 40.14 |
| age | | | | | | | | | |
| wt | 11.61 | 0.67 | 2.78 | 0.89 | 13.77 | 0.86 | 2.60 | 11.08 | 42.79 |
| #1 | | | | | | | | | |
| wt | 11.79 | 0.74 | 2.61 | 0.93 | 12.76 | 0.92 | 3.46 | 12.75 | 41.84 |
| #2 | | | | | | | | | |
| wt | 11.62 | 0.89 | 2.44 | 1.06 | 12.64 | 0.99 | 4.00 | 12.82 | 40.45 |
| #3 | | | | | | | | | |
| wt | 11.57 | 0.79 | 2.57 | 0.92 | 12.47 | 0.88 | 3.56 | 11.69 | 41.60 |
| #4 | | | | | | | | | |
| wt | 11.63 | 0.85 | 2.55 | 1.07 | 11.46 | 1.07 | 4.15 | 13.66 | 39.67 |
| #5 | | | | | | | | | |
| Aver- | 11.644 | 0.788 | 2.59 | 0.974 | 12.62 | 0.944 | 3.554 | 12.4 | 41.27 |
| age | | | | | | | | | |

All publications and patents mentioned in the above specification are herein incorporated by reference. Various modifications and variations of the described compositions and methods of the invention will be apparent to those skilled in the art without departing from the scope and spirit of the invention. Although the invention has been described in connection with particular preferred embodiments, it should be understood that the inventions claimed should not be unduly limited to such specific embodiments. Indeed, various modifications of the described modes for carrying out the invention which are obvious to those skilled in the art and in fields related thereto are intended to be within the scope of the following claims.

What is claimed is:

1. An isolated nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO: 11.

2. The nucleic acid sequence of claim 1, wherein said sequence is operably linked to a heterologous promoter.

3. The nucleic acid sequence of claim 1, wherein said sequence is contained within a vector.

4. The nucleic acid sequence of claim 2, wherein said nucleic acid sequence is within a host cell.

5. An isolated nucleic acid sequence encoding a protein comprising an amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, and SEQ ID NO:22.

6. A method for altering the phenotype of a plant comprising:
a) providing:
i) a vector comprising a nucleic acid sequence encoding a protein, said nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO: 11; and
ii) plant tissue; and
b) transfecting said plant tissue with said vector under conditions such that said protein is expressed.

7. A method for altering the phenotype of a plant comprising:
a) providing:
i) a vector comprising a nucleic acid sequence encoding a protein, wherein said protein comprises an amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, and SEQ ID NO:22; and
ii) plant tissue; and
b) transfecting said plant tissue with said vector under conditions such that said protein is expressed.

8. A method for assaying acyl-CoA synthetase activity comprising:
a) providing a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11;
b) expressing said nucleic acid sequence under conditions such that a protein is produced; and
c) assaying the activity of said protein.

9. A method for assaying acyl-CoA synthetase activity comprising:
a) providing a nucleic acid sequence encoding a protein comprising an amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, and SEQ ID NO:22;
b) expressing said nucleic acid sequence under conditions such that a protein is produced; and
c) assaying the activity of said protein.

10. An isolated nucleic acid sequence that hybridizes under conditions of low stringency to a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11, wherein said nucleic acid sequence is operably linked to a heterologous promoter.

11. The nucleic acid sequence of claim 10, wherein said sequence encodes a protein that catalyzes the esterification of a fatty acid and coenzyme A.

12. A method for altering the phenotype of a plant comprising:
a) providing:
i) a vector comprising a nucleic acid sequence encoding a protein, said nucleic acid sequence selected from the group consisting of sequences that hybridize under low stringency conditions to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11; and
ii) plant tissue; and
b) transfecting said plant tissue with said vector under conditions such that said protein is expressed.

13. A host cell comprising a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11 and sequences that hybridize under low stringency conditions to SEQ ID NOs:1-11, wherein said nucleic acid sequence is operably linked to an exogenous promoter.

14. A transgenic plant comprising a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11, and sequences that hybridize under low stringency conditions to SEQ ID NOs:1-11, wherein said nucleic acid sequence is operably linked to an exogenous promoter.

15. Oil from the transgenic plant of claim 14.

16. Seeds from the transgenic plant of claim 14.

17. A method for altering the phenotype of a plant comprising:
a) providing:
i) a vector comprising an antisense sequence corresponding to a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11; and
ii) plant tissue; and
b) transfecting said plant tissue with said vector under conditions such that said antisense sequence is expressed and the activity of an acyl-CoA synthetase is down regulated as compared to wild-type plants.

18. A method for producing variants of acyl-CoA synthetases comprising:
a) providing a nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO: 11;
b) mutagenizing said nucleic acid sequence; and
c) screening a variant encoded by the mutagenized nucleic acid sequence for activity.

19. A method for screening acyl CoA synthetases comprising:
a) providing a candidate acyl-CoA synthetase; and
b) analyzing said candidate acyl-CoA synthetase for the presence of at least one of ACS motifs 1-9.

20. An isolated nucleic acid sequence encoding a plant acyl-CoA synthetase, wherein said plant acyl CoA synthetase competes for binding to a fatty acid substrate with a protein encoded by a second nucleic acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

21. A composition comprising a first nucleic acid sequence, wherein the first nucleic acid sequence inhibits the binding of at least a portion of a second nucleic acid sequence to its complementary sequence and wherein the second nucleic acid sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:11.

22. A purified protein comprising an amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, and SEQ ID NO:22.

23. A protein produced by expressing a nucleic acid sequence of claim 2.

24. A protein produced by expressing a nucleic acid sequence of claim 5, wherein said nucleic acid sequence is operably linked to a heterologous promoter.

25. A composition comprising a purified protein comprising an amino acid sequence selected from the group consisting of SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, and SEQ ID NO:22.
